(19) United States
(12) Patent Application Publication
(10) Pub. No.: US 2022/0175003 A1
(43) Pub. Date: Jun. 9, 2022

(54) MICROWAVE ASSISTED EXTRACTION OF ESSENTIAL OILS FROM PLANT BIOMASS

(71) Applicant: FIRMENICH SA, Satigny (CH)

(72) Inventors: Xavier Brochet, Grasse (FR); Philippe Pineau, Grasse (FR); Stephanie Salord, Satigny (CH); Yann Lemasson, Grasse (FR); Patrick Salord, Satigny (CH); Angela Di Pietro, Satigny (CH); Brigitte Pellen, Grasse (FR)

(21) Appl. No.: 17/434,299

(22) PCT Filed: Feb. 25, 2020
(86) PCT No.: PCT/EP2020/054950

§ 371 Date: Aug. 26, 2021

Related U.S. Application Data
(60) Provisional application No. 62/810,650, filed on Feb. 26, 2019.

(30) Foreign Application Priority Data
May 16, 2019 (EP) 19174970.4

(51) Int. Cl. A23L 33/105; C11B 9/02; A23L 27/12; A61K 8/49; A61K 8/60

(52) U.S. Cl. CPC A23L 33/105; C11B 9/02; A61K 8/60; A61K 8/4973; A23L 27/12

(57) ABSTRACT

The aspects presented herein provide essential oils, extracts, apparatus and methods for the extraction of the essential oils and extracts from plant biomass using microwaves.

### FIELD OF THE INVENTION

The present disclosure relates to essential oils, extracts, apparatus and methods for the extraction of the essential oils and extracts from plant biomass using microwaves.

### BACKGROUND

Microwave assisted extraction of essential oils from plant biomass has had limited commercial development. Problems encountered in scaling up microwave assisted extraction methods up to an industrial scale include, for example, consistently obtaining essential oils from large quantities of plant biomass. Accordingly, there is a need for microwave assisted extraction methods at an industrial scale.

### SUMMARY

One aspect presented herein provides an extract of a plant biomass, wherein the extract comprises at least one anthocyanin. In one aspect, the extract further comprises at least one anthocyanidin.

In one aspect, the concentration of the at least one anthocyanin in the extract is 1 ppm.

In one aspect, the concentration of the at least one anthocyanidin in the extract is 1 ppm.

In one aspect, the at least one anthocyanidin is selected from the group consisting of: cyanidin, delphinidin, malvinidin, peonidin, petunidin, and combinations thereof.

In one aspect, the at least one anthocyanin is selected from the group consisting of: cyanidin-3-arabinoside, cyanidin-3-galactoside, cyanidin-3-glucoside, delphinidin-3-arabinoside, delphinidin-3-galactoside, delphinidin-3-glucoside, malvinidin-3-arabinoside, malvinidin-3-galactoside, malvinidin-3-glucoside, peonidin-3-arabinoside, peonidin-3-galactoside, peonidin-3-glucoside, petunidin-3-arabinoside, petunidin-3-galactoside, petunidin-3-glucoside, and combinations thereof.

In one aspect, the concentration of cyanidin-3-glucoside in the extract is 0.5 ppm.

In one aspect, the concentration of peonidin in the extract is 0.07 ppm.

In one aspect, the concentration of peonidin-3-galactoside in the extract is 0.2 ppm.

In one aspect, the concentration of peonidin-3-glucoside in the extract is 0.8 ppm.

In one aspect, the concentration of petunidin in the extract is 0.6 ppm.

In one aspect, the extract comprises 0.5 ppm cyanidin-3-glucoside, 0.07 ppm peonidin, 0.2 ppm peonidin-3-galactoside, 0.8 ppm peonidin-3-glucoside, and 0.6 ppm petunidin.

In one aspect, the extract comprises glucose in an amount greater than 1 g/100 g of the extract.

In one aspect, the extract comprises glucose in an amount from 0.9 to 1.1 g/100 g of the extract.

In one aspect, the extract comprises fructose in an amount from 1.1 to 1.5 g/100 g of the extract.

In one aspect, the extract comprises sucrose in an amount from 0.2 to 1 g/100 g of the extract.

In one aspect, the extract comprises reducing sugars in an amount from 2 to 2.5 g/100 g of the extract.

In one aspect, the extract comprises mono and disaccharides in an amount from 2 to 3.5 g/100 g of the extract.

In one aspect, the plant biomass is strawberry.

In one aspect the invention provides a strawberry provides a strawberry extract comprising one or more of the following features: (i) 0.007 ppm to 0.027 ppm 2,3 butanedione, (ii) 0.05 ppm to 0.1 ppm 3,7,11-trimethyl-1,6,10-Dodecatrien-3-ol; (iii) 0.02 to 0.1 ppm dihydro-5-octyl-2(3H)-Furanone; (iv) 0.05 to 0.15 ppm 4-hydroxy-2,5-dimethyl-3(2H)-Furanone.

A further aspect of the invention provides a strawberry extract comprising one or more of the following features: (i) less than 0.3 ppm 2-methylpropan-1-ol; (ii) less than 0.5 ppm 3-methyl butanol; (iii) less than 0.4 ppm 2-hexenal.

A further aspect of the invention provides an strawberry extract comprising: (i) 0.007 ppm to 0.027 ppm 2,3 butanedione, (ii) 0.05 ppm to 0.1 ppm 3,7,11-trimethyl-1,6,10-Dodecatrien-3-ol; (iii) 0.02 to 0.1 ppm dihydro-5-octyl-2(3H)-Furanone; (iv) 0.05 to 0.15 ppm 4-hydroxy-2,5-dimethyl-3(2H)-Furanone, (v) less than 0.3 ppm 2-methylpropan-1-ol; (vi) less than 0.5 ppm 3-methyl butanol; and (vii) less than 0.4 ppm 2-hexenal.

One aspect presented herein provides a perfumed article containing the extract according to some aspects presented herein, wherein the perfumed article is selected from the group consisting of: an aromatic water, a perfume, a cologne, a bath gel, a shower gel, a shampoo, a hair care product, a cosmetic preparation, a deodorant, an air freshener, a detergent, a fabric softener, and a household cleaner.

One aspect presented herein provides a flavouring composition. containing the extract. The flavouring composition may be incorporated into a flavored consumer product composition such as, for example, foodstuffs beverages and beverage products.

### BRIEF DESCRIPTION OF THE FIGURES

FIG. 1 shows a representation of apparatus for extracting essential oils from plant biomass according to some aspects presented herein.

FIG. 2 shows a representation of apparatus for extracting essential oils from plant biomass according to some aspects presented herein.

FIG. 3 shows a representation of a method according to some aspects presented herein.

FIG. 4 shows an iso intensity sensory profile of strawberry classical extract versus a strawberry extract using the methods of the invention.

FIG. 5 shows an iso intensity sensory profile of strawberry classical extract versus a strawberry extract using the methods of the invention in typical Strawberry beverage.

FIG. 5 shows an iso intensity sensory profile of strawberry classical extract versus a strawberry extract using the methods of the invention in typical Strawberry beverage.

FIG. 6 shows an iso intensity sensory profile of Strawberry Juicy Water containing 5% juice from Conc. (57 Brix) vs one containing 5% Juice from conc. (65 Brix) and vs containing strawberry extract using the methods of the invention

### DETAILED DESCRIPTION

In the following description, reference is made to specific embodiments which may be practiced, which is shown by way of illustration. These embodiments are described in detail to enable those skilled in the art to practice the invention described herein, and it is to be understood that other embodiments may be utilized and that logical changes may be made without departing from the scope of the aspects presented herein. The following description of example embodiments is, therefore, not to be taken in a limited sense, and the scope of the various aspects presented herein is defined by the appended claims.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

### The Method According to Some Aspects Presented Herein

Without intending to be limited to any particular theory, conventional methods to obtain extracts, such as, for example, essential oils from plant biomass frequently require the use of solvents that may be harmful to the environment, and/or humans. Furthermore, conventional methods frequently require large volumes of solvent to achieve acceptable yields of the extract. While microwave assisted extraction may overcome some of the limitations of conventional extraction methods, microwave assisted methods of obtaining extracts, such as, essential oils from plant biomass has had limited commercial development. Problems encountered in scaling up microwave assisted extraction methods up to an industrial scale include, for example, consistently obtaining extracts from a large volume of plant biomass. Additionally, the olfactive properties of the extract are frequently altered by the extraction process. Alterations may be caused by several factors, including, but not limited to the presence, or enrichment of compounds having undesirable olfactive notes. Other alterations may be the absence, or reduction of compounds having the desired olfactive notes.

Consequently, there is a need for microwave assisted extraction methods at an industrial scale that are capable of generating an extract, such as an essential oil with improved olfactive properties.

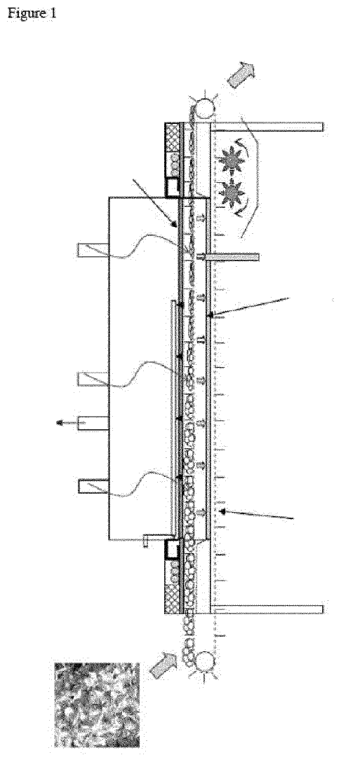

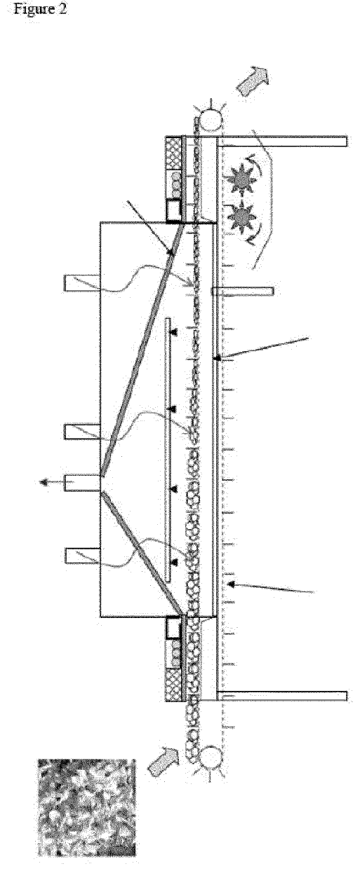

Referring to FIGS. 1 and 2, the present disclosure provides a continuous extraction method, wherein plant biomass is continually introduced into a vessel at one end, and subjected to microwave energy to vaporize the water present in the biomass, thereby producing a distillate. In some aspects, the distillate contains the desired extract. In some aspects, the extract is an essential oil. In the aspects shown in FIGS. 1 and 2, the treated plant biomass is expelled from the vessel at the opposite end.

The apparatus shown in FIGS. 1 and 2 may be scaled up. For example, the length of the conveyor belt, the length of the vessel, and/or the velocity of the conveyor belt may be increased, or decreased, depending on the quantity of the plant biomass to be processed and/or the desired volume of the extract.

Without intending to be limited to any particular theory, the methods and apparatus presented herein do not require the addition of solvents, and produce extracts, essential oils having improved olfactive profiles. In some aspects, the improved olfactive profile may be an olfactive profile the same, or highly similar to the fresh plant. Alternatively, the improved olfactive profile may be an olfactive profile having fewer artefacts, such as, for example, undesired olfactive notes.

The apparatus shown in FIGS. 1 and 2 may be modified. Modifications include configurations to allow classical hydro-distillation. Another example of a modification may be a configuration to allow collection of the distillate via gravimetry. Another example of a modification may be altering the confirmation of the conveyor belt. Examples of such alterations include, but are not limited to increasing the porosity of the conveyer belt, and the like.

Another example of a modification may be the application of exogenous steam. In some aspects, the flow of steam may also be modified.

Another example of a modification may be the recycling of the distillate within the vessel, such as, for example, via cohobation, to concentrate the distillate. An example of cohobation is disclosed in French Patent Application Publication No. FR3003181 A1.

Another example of a modification may be a depression in the inner vessel, wherein the depression allows the distillate to collect and be removed by gravimetry.

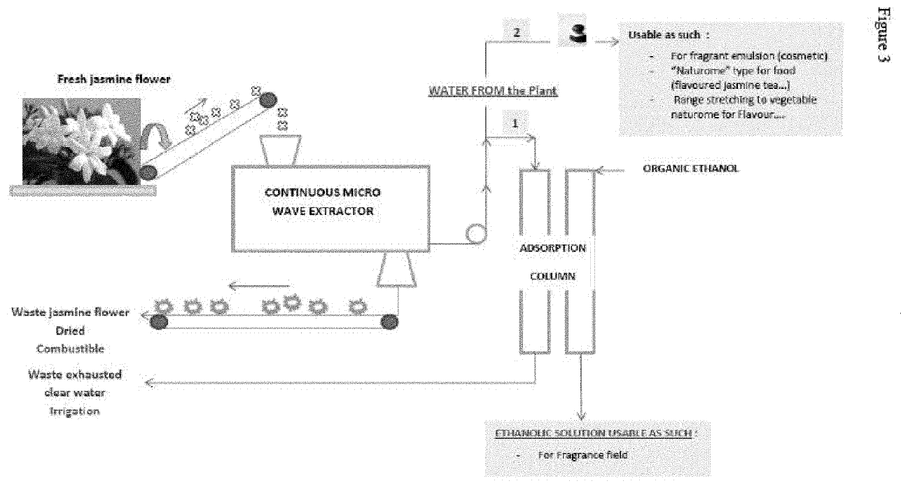

Referring to FIG. 3, in some aspects, the present disclosure provides a process, wherein plant biomass is introduced into a vessel and subjected to microwave energy to vaporize the water present in the biomass, thereby producing a distillate. In the aspect shown in FIG. 3, the distillate is collected by gravimetry.

In some aspects, the distillate is used, without any additional processing. Alternatively, in some aspects, the distillate is further processed.

Examples of further processing include, but are not limited to, concentration, drying, thin-film distillation, resin-based chromatography, and the like.

In some aspects, the essential oils are purified from the distillate by decanting the oil from the aqueous phase of the distillate.

In some aspects, the distillate is adsorbed onto a column, and eluted off with a solvent, with a flow of 3 to 10 bed volumes per hour.

In some aspects, the flow may be from 4 to 10, alternatively, from 5 to 10, alternatively, from 6 to 10, alternatively, from 7 to 10, alternatively, from 8 to 10, alternatively, from 9 to 10 bed volumes per hour.

In some aspects, the flow may be from 4 to 9, alternatively, from 4 to 8, alternatively, from 4 to 7, alternatively, from 4 to 6, alternatively, from 4 to 5 bed volumes per hour.

In some aspects, the flow may be 3, or 4, or 5, or 6, or 7, or 8, or 9, or 10 bed volumes per hour.

For example, in the aspect presented in FIG. 3, the distillate is adsorbed onto a XAD4 resin column, and eluted off with ethanol, with a flow of 5 bed volumes per hour.

Other solvents include but are not limited to, steam, supercritical CO₂, and the like.

Other resins include, but are not limited to XAD16, and the like.

In some aspects, the apparatus and methods disclosed herein provide a method for the continuous extraction, not batch-based extraction of extracts, such as, for example, essential oils from plant biomass. Without intending to be limited to any particular theory, the apparatus and methods disclosed result in smaller lot to lot variation of the extract. Moreover, the parameters of the apparatus and methods disclosed herein may be adjusted to alter the properties of the distillate, and thus the extract obtained. Adjustments include, but are not limited to, the first velocity of the conveyer belt, the length of the conveyer belt, the first intensity of the microwave energy, the length of the vessel, the temperature within the vessel, the rate of addition of the plant biomass, the time sufficient to treat the plant biomass, or any combination thereof.

Without intending to be limited to any particular theory, the methods and apparatus presented herein may produce a smaller amount of non-recyclable waste, compared to other methods in the art. In addition, the methods and apparatus presented herein may have a smaller CO₂ footprint, compared to other methods in the art.

Additionally, the methods and apparatus presented herein may be more energy efficient, compared to other methods in the art.

Additionally, the methods and apparatus presented herein may produce essential oils faster, compared to other methods in the art.

Accordingly, some presented herein provides a method,
wherein the method obtains an extract from plant biomass,
wherein the method comprises the steps of:
   a. obtaining the plant biomass;
   b. subjecting the plant biomass to microwave energy, of a first intensity, for a time sufficient to heat the biomass to a temperature to vaporize water within the biomass, thereby producing a distillate; and
   c. collecting the distillate.

Some aspects presented herein provide a method,
wherein the method obtains an extract from plant biomass,
wherein the method comprises the steps of:
   a. obtaining the plant biomass;
   b. placing the plant biomass onto a conveyor belt, having a length, and introducing the plant biomass into a vessel having length, via the conveyor belt,
      wherein the plant biomass is introduced into the vessel by moving the conveyer belt at a first velocity, and
      wherein the plant biomass is introduced into the vessel at a rate;
   c. subjecting the plant biomass to microwave energy, of a first intensity, for a time sufficient to heat the biomass to a temperature to vaporize water within the biomass, thereby producing a distillate; and
   d. collecting the distillate.

In some aspects, the conveyer belt comprises pores. In some aspects, the pores are configured to introduce finely ground plant biomass into the vessel.

In some aspects, the extract comprises an essential oil.

In some aspects, the time sufficient to heat the biomass to a temperature to vaporize water is controlled by the first velocity of the conveyer belt, the length of the conveyer belt, the first intensity of the microwave energy, the length of the vessel, or any combination thereof.

Accordingly, in some aspects, the length of the conveyor belt is at least 1 meter. In some aspects, the length of the conveyor belt is at least 1.5, or at least 2, or at least 2.5, or at least 3, or at least 3.5, or at least 4, or at least 4.5, or at least 5, or at least 5.5, or at least 6, or at least 6.5, or at least 7, or at least 7.5, or at least 8, or at least 8.5, or at least 9, or at least 9.5, or at least 10 meters.

Similarly, in some aspects, the length of the vessel is at least 1 meter. In some aspects, the length of the vessel is at least 1.5, or at least 2, or at least 2.5, or at least 3, or at least 3.5, or at least 4, or at least 4.5, or at least 5, or at least 5.5, or at least 6, or at least 6.5, or at least 7, or at least 7.5, or at least 8, or at least 8.5, or at least 9, or at least 9.5, or at least 10 meters.

In some aspects, the plant biomass is introduced at a rate greater than or equal to 12 Kg/hour. In some aspects, the plant biomass is introduced at a rate from 0.5 to 12 Kg/hour. In some aspects, the plant biomass is introduced at a rate from 0.5 to 11.5 Kg/hour. In some aspects, the plant biomass is introduced at a rate from 0.5 to 11 Kg/hour. In some aspects, the plant biomass is introduced at a rate from 0.5 to 10.5 Kg/hour. In some aspects, the plant biomass is introduced at a rate from 0.5 to 10 Kg/hour. In some aspects, the plant biomass is introduced at a rate from 0.5 to 9.5 Kg/hour. In some aspects, the plant biomass is introduced at a rate from 0.5 to 9 Kg/hour. In some aspects, the plant biomass is introduced at a rate from 0.5 to 8.5 Kg/hour. In some aspects, the plant biomass is introduced at a rate from 0.5 to 8 Kg/hour. In some aspects, the plant biomass is introduced at a rate from 0.5 to 7.5 Kg/hour. In some aspects, the plant biomass is introduced at a rate from 0.5 to 7 Kg/hour. In some aspects, the plant biomass is introduced at a rate from 0.5 to 6.5 Kg/hour. In some aspects, the plant biomass is introduced at a rate from 0.5 to 6 Kg/hour. In some aspects, the plant biomass is introduced at a rate from 0.5 to 5.5 Kg/hour. In some aspects, the plant biomass is introduced at a rate from 0.5 to 5 Kg/hour. In some aspects, the plant biomass is introduced at a rate from 0.5 to 4.5 Kg/hour. In some aspects, the plant biomass is introduced at a rate from 0.5 to 4 Kg/hour. In some aspects, the plant biomass is introduced at a rate from 0.5 to 3.5 Kg/hour. In some aspects, the plant biomass is introduced at a rate from 0.5 to 3 Kg/hour. In some aspects, the plant biomass is introduced at a rate from 0.5 to 2.5 Kg/hour. In some aspects, the plant biomass is introduced at a rate from 0.5 to 2 Kg/hour. In some aspects, the plant biomass is introduced at a rate from 0.5 to 1.5 Kg/hour. In some aspects, the plant biomass is introduced at a rate from 0.5 to 1 Kg/hour.

In some aspects, the plant biomass is introduced at a rate from 1 to 12 Kg/hour. In some aspects, the plant biomass is introduced at a rate from 1.5 to 12 Kg/hour. In some aspects, the plant biomass is introduced at a rate from 2 to 12 Kg/hour. In some aspects, the plant biomass is introduced at a rate from 2.5 to 12 Kg/hour. In some aspects, the plant biomass is introduced at a rate from 3 to 12 Kg/hour. In some aspects, the plant biomass is introduced at a rate from 3.5 to 12 Kg/hour. In some aspects, the plant biomass is introduced at a rate from 4 to 12 Kg/hour. In some aspects, the plant biomass is introduced at a rate from 4.5 to 12 Kg/hour. In some aspects, the plant biomass is introduced at a rate from 5 to 12 Kg/hour. In some aspects, the plant biomass is introduced at a rate from 5.5 to 12 Kg/hour. In some aspects, the plant biomass is introduced at a rate from 6 to 12 Kg/hour. In some aspects, the plant biomass is introduced at a rate from 6.5 to 12 Kg/hour. In some aspects, the plant biomass is introduced at a rate from 7 to 12 Kg/hour. In some aspects, the plant biomass is introduced at a rate from 7.5 to 12 Kg/hour. In some aspects, the plant biomass is introduced at a rate from 8 to 12 Kg/hour. In some aspects, the plant biomass is introduced at a rate from 8.5 to 12 Kg/hour. In some aspects, the plant biomass is introduced at a rate from 9 to 12 Kg/hour. In some aspects, the plant biomass is introduced at a rate from 9.5 to 12 Kg/hour. In some aspects, the plant biomass is introduced at a rate from 10 to 12 Kg/hour. In some aspects, the plant biomass is introduced at a rate from 10.5 to 12 Kg/hour. In some aspects, the plant biomass is introduced at a rate from 11 to 12 Kg/hour. In some aspects, the plant biomass is introduced at a rate from 11.5 to 12 Kg/hour.

In some aspects, the plant biomass is introduced at 0.5, or 1, or 1.5, or 2, or 2.5, or 3, or 3.5, or 4, or 4.5, or 5, or 5.5, or 6, or 6.5, or 7, or 7.5, or 8, or 8.5, or 9, or 9.5, or 10, or 10.5, or 11, or 11.5, or 12 Kg per hour.

In some aspects, the plant biomass is introduced into the vessel at a rate of at least 4 Kg of plant biomass per hour.

In some aspects, the time sufficient is from 20 to 45 minutes. In some aspects, the time sufficient is from 25 to 45 minutes. In some aspects, the time sufficient is from 30 to 45 minutes. In some aspects, the time sufficient is from 35 to 45 minutes. In some aspects, the time sufficient is from 40 to 45 minutes.

In some aspects, the time sufficient is from 20 to 40 minutes. In some aspects, the time sufficient is from 25 to 35 minutes. In some aspects, the time sufficient is from 20 to 30 minutes. In some aspects, the time sufficient is from 20 to 25 minutes.

In some aspects, the time sufficient is 20, or 25, or 30, or 35, or 40, or 45 minutes.

In some aspects, the first intensity of the microwave energy is from 100 to 3000 Watts. In some aspects, the first intensity of the microwave energy is from 100 to 2900 Watts. In some aspects, the first intensity of the microwave energy is from 100 to 2800 Watts. In some aspects, the first intensity of the microwave energy is from 100 to 2700 Watts. In some aspects, the first intensity of the microwave energy is from 100 to 2600 Watts. In some aspects, the first intensity of the microwave energy is from 100 to 2500 Watts. In some aspects, the first intensity of the microwave energy is from 100 to 2400 Watts. In some aspects, the first intensity of the microwave energy is from 100 to 2300 Watts. In some aspects, the first intensity of the microwave energy is from 100 to 2200 Watts. In some aspects, the first intensity of the microwave energy is from 100 to 2100 Watts. In some aspects, the first intensity of the microwave energy is from 100 to 2000 Watts. In some aspects, the first intensity of the microwave energy is from 100 to 1900 Watts. In some aspects, the first intensity of the microwave energy is from 100 to 1800 Watts. In some aspects, the first intensity of the microwave energy is from 100 to 1700 Watts. In some aspects, the first intensity of the microwave energy is from 100 to 1600 Watts. In some aspects, the first intensity of the microwave energy is from 100 to 1500 Watts. In some aspects, the first intensity of the microwave energy is from 100 to 1400 Watts. In some aspects, the first intensity of the microwave energy is from 100 to 1300 Watts. In some aspects, the first intensity of the microwave energy is from 100 to 1200 Watts. In some aspects, the first intensity of the microwave energy is from 100 to 1100 Watts. In some aspects, the first intensity of the microwave energy is from 100 to 1000 Watts. In some aspects, the first intensity of the microwave energy is from 100 to 900 Watts. In some aspects, the first intensity of the microwave energy is from 100 to 800 Watts. In some aspects, the first intensity of the microwave energy is from 100 to 700 Watts. In some aspects, the first intensity of the microwave energy is from 100 to 600 Watts. In some aspects, the first intensity of the microwave energy is from 100 to 500 Watts. In some aspects, the first intensity of the microwave energy is from 100 to 400 Watts. In some aspects, the first intensity of the microwave energy is from 100 to 300 Watts. In some aspects, the first intensity of the microwave energy is from 100 to 200 Watts.

In some aspects, the first intensity of the microwave energy is from 200 to 3000 Watts. In some aspects, the first intensity of the microwave energy is from 300 to 3000 Watts. In some aspects, the first intensity of the microwave energy is from 400 to 3000 Watts. In some aspects, the first intensity of the microwave energy is from 500 to 3000 Watts. In some aspects, the first intensity of the microwave energy is from 600 to 3000 Watts. In some aspects, the first intensity of the microwave energy is from 700 to 3000 Watts. In some aspects, the first intensity of the microwave energy is from 800 to 3000 Watts. In some aspects, the first intensity of the microwave energy is from 900 to 3000 Watts. In some aspects, the first intensity of the microwave energy is from 1000 to 3000 Watts. In some aspects, the first intensity of the microwave energy is from 1100 to 3000 Watts. In some aspects, the first intensity of the microwave energy is from 1200 to 3000 Watts. In some aspects, the first intensity of the microwave energy is from 1300 to 3000 Watts. In some aspects, the first intensity of the microwave energy is from 1400 to 3000 Watts. In some aspects, the first intensity of the microwave energy is from 1500 to 3000 Watts. In some aspects, the first intensity of the microwave energy is from 1600 to 3000 Watts. In some aspects, the first intensity of the microwave energy is from 1700 to 3000 Watts. In some aspects, the first intensity of the microwave energy is from 1800 to 3000 Watts. In some aspects, the first intensity of the microwave energy is from 1900 to 3000 Watts. In some aspects, the first intensity of the microwave energy is from 2000 to 3000 Watts. In some aspects, the first intensity of the microwave energy is from 2100 to 3000 Watts. In some aspects, the first intensity of the microwave energy is from 2200 to 3000 Watts. In some aspects, the first intensity of the microwave energy is from 2300 to 3000 Watts. In some aspects, the first intensity of the microwave energy is from 2400 to 3000 Watts. In some aspects, the first intensity of the microwave energy is from 2500 to 3000 Watts. In some aspects, the first intensity of the microwave energy is from 2600 to 3000 Watts. In some aspects, the first intensity of the microwave energy is from 2700 to 3000 Watts. In some aspects, the first intensity of the microwave energy is from 2800 to 3000 Watts. In some aspects, the first intensity of the microwave energy is from 2900 to 3000 Watts.

In some aspects, the first intensity of the microwave energy is 200, or 300, or 400, or 500, or 600, or 700, or 800, or 900, or 1000, or 1100, or 1200, or 1300, or 1400, or 1500, or 1600, or 1700, or 1800, or 1900, or 2000, or 2100, or 2200, or 2300, or 2400, or 2500, or 2600, or 2700, or 2800, or 2900, or 3000 Watts.

In some aspects, the first intensity of the microwave energy is 200 Watts. In some aspects, the first intensity is less than 200 Watts. In some aspects, the first intensity is an intensity sufficient to vaporize water within the biomass.

In some aspects, the first intensity of the microwave energy is from 0.2 KWH/Kg biomass to 0.6 KWH/Kg biomass. In some aspects, the first intensity of the microwave energy is from 0.25 KWH/Kg biomass to 0.6 KWH/Kg biomass. In some aspects, the first intensity of the microwave energy is from 0.3 KWH/Kg biomass to 0.6 KWH/Kg biomass. In some aspects, the first intensity of the microwave energy is from 0.35 KWH/Kg biomass to 0.6 KWH/Kg biomass. In some aspects, the first intensity of the microwave energy is from 0.4 KWH/Kg biomass to 0.6 KWH/Kg biomass. In some aspects, the first intensity of the microwave energy is from 0.45 KWH/Kg biomass to 0.6 KWH/Kg biomass. In some aspects, the first intensity of the microwave energy is from 0.3 KWH/Kg biomass to 0.5 KWH/Kg biomass. In some aspects, the first intensity of the microwave energy is from 0.55 KWH/Kg biomass to 0.6 KWH/Kg biomass.

In some aspects, the first intensity of the microwave energy is from 0.2 KWH/Kg biomass to 0.55 KWH/Kg biomass. In some aspects, the first intensity of the microwave energy is from 0.2 KWH/Kg biomass to 0.5 KWH/Kg biomass. In some aspects, the first intensity of the microwave energy is from 0.2 KWH/Kg biomass to 0.45 KWH/Kg biomass. In some aspects, the first intensity of the microwave energy is from 0.2 KWH/Kg biomass to 0.4 KWH/Kg biomass. In some aspects, the first intensity of the microwave energy is from 0.2 KWH/Kg biomass to 0.35 KWH/Kg biomass. In some aspects, the first intensity of the microwave energy is from 0.2 KWH/Kg biomass to 0.3 KWH/Kg biomass. In some aspects, the first intensity of the microwave energy is from 0.2 KWH/Kg biomass to 0.25 KWH/Kg biomass.

In some aspects, the first intensity of the microwave energy is 0.2, or 0.25, or 0.3, or 0.35, or 0.4, or 0.45, or 0.5, or 0.55, or 0.6 KWH/Kg biomass.

In some aspects, the first intensity may be either increased, or alternatively, decreased to a second intensity during the method.

In some aspects, the temperature of the inside of the vessel is from 40 to 100 degrees Celsius. In some aspects, the temperature of the inside of the vessel is from 40 to 90 degrees Celsius. In some aspects, the temperature of the inside of the vessel is from 40 to 80 degrees Celsius. In some aspects, the temperature of the inside of the vessel is from 40 to 70 degrees Celsius. In some aspects, the temperature of the inside of the vessel is from 40 to 60 degrees Celsius. In some aspects, the temperature of the inside of the vessel is from 40 to 50 degrees Celsius.

In some aspects, the temperature of the inside of the vessel is from 50 to 100 degrees Celsius. In some aspects, the temperature of the inside of the vessel is from 60 to 100 degrees Celsius. In some aspects, the temperature of the inside of the vessel is from 70 to 100 degrees Celsius. In some aspects, the temperature of the inside of the vessel is from 80 to 100 degrees Celsius. In some aspects, the temperature of the inside of the vessel is from 90 to 100 degrees Celsius.

In some aspects, the temperature of the inside of the vessel is from 60 to 80 degrees Celsius.

In some aspects, the temperature of the inside of the vessel is 40, or 50, or 60, or 70, or 80, or 90, or 100 degrees Celsius.

In some aspects the temperature of the inside of the vessel may be either increased, or alternatively, decreased to a second temperature during the method.

In some aspects, the collected distillate comprises an aqueous extract. In some aspects, the collected distillate comprises an essential oil. In some aspects, the essential oil is obtained from the aqueous extract.

In some aspects, the collected distillate comprises from 200 to 3000 ppm essential oils. In some aspects, the collected distillate comprises from 200 to 2900 ppm essential oils. In some aspects, the collected distillate comprises from 200 to 2800 ppm essential oils. In some aspects, the collected distillate comprises from 200 to 2700 ppm essential oils. In some aspects, the collected distillate comprises from 200 to 2600 ppm essential oils. In some aspects, the collected distillate comprises from 200 to 2500 ppm essential oils. In some aspects, the collected distillate comprises from 200 to 2400 ppm essential oils. In some aspects, the collected distillate comprises from 200 to 2300 ppm essential oils. In some aspects, the collected distillate comprises from 200 to 2200 ppm essential oils. In some aspects, the collected distillate comprises from 200 to 2100 ppm essential oils. In some aspects, the collected distillate comprises from 200 to 2000 ppm essential oils. In some aspects, the collected distillate comprises from 200 to 1900 ppm essential oils. In some aspects, the collected distillate comprises from 200 to 1800 ppm essential oils. In some aspects, the collected distillate comprises from 200 to 1700 ppm essential oils. In some aspects, the collected distillate comprises from 200 to 1600 ppm essential oils. In some aspects, the collected distillate comprises from 200 to 1500 ppm essential oils. In some aspects, the collected distillate comprises from 200 to 1400 ppm essential oils. In some aspects, the collected distillate comprises from 200 to 1300 ppm essential oils. In some aspects, the collected distillate comprises from 200 to 1200 ppm essential oils. In some aspects, the collected distillate comprises from 200 to 1100 ppm essential oils. In some aspects, the collected distillate comprises from 200 to 1000 ppm essential oils. In some aspects, the collected distillate comprises from 200 to 900 ppm essential oils. In some aspects, the collected distillate comprises from 200 to 800 ppm essential oils. In some aspects, the collected distillate comprises from 200 to 700 ppm essential oils. In some aspects, the collected distillate comprises from 200 to 600 ppm essential oils. In some aspects, the collected distillate comprises from 200 to 500 ppm essential oils. In some aspects, the collected distillate comprises from 200 to 400 ppm essential oils. In some aspects, the collected distillate comprises from 200 to 300 ppm essential oils.

In some aspects, the collected distillate comprises from 300 to 3000 ppm essential oils. In some aspects, the collected distillate comprises from 400 to 3000 ppm essential oils. In some aspects, the collected distillate comprises from 500 to 3000 ppm essential oils. In some aspects, the collected distillate comprises from 600 to 3000 ppm essential oils. In some aspects, the collected distillate comprises from 700 to 3000 ppm essential oils. In some aspects, the collected distillate comprises from 800 to 3000 ppm essential oils. In some aspects, the collected distillate comprises from 900 to 3000 ppm essential oils. In some aspects, the collected distillate comprises from 1000 to 3000 ppm essential oils. In some aspects, the collected distillate comprises from 1100 to 3000 ppm essential oils. In some aspects, the collected distillate comprises from 1200 to 3000 ppm essential oils. In some aspects, the collected distillate comprises from 1300 to 3000 ppm essential oils. In some aspects, the collected distillate comprises from 1400 to 3000 ppm essential oils. In some aspects, the collected distillate comprises from 1500 to 3000 ppm essential oils. In some aspects, the collected distillate comprises from 1600 to 3000 ppm essential oils. In some aspects, the collected distillate comprises from 1700 to 3000 ppm essential oils. In some aspects, the collected distillate comprises from 1800 to 3000 ppm essential oils. In some aspects, the collected distillate comprises from 1900 to 3000 ppm essential oils. In some aspects, the collected distillate comprises from 2000 to 3000 ppm essential oils. In some aspects, the collected distillate comprises from 2100 to 3000 ppm essential oils. In some aspects, the collected distillate comprises from 2200 to 3000 ppm essential oils. In some aspects, the collected distillate comprises from 2300 to 3000 ppm essential oils. In some aspects, the collected distillate comprises from 2400 to 3000 ppm essential oils. In some aspects, the collected distillate comprises from 2500 to 3000 ppm essential oils. In some aspects, the collected distillate comprises from 2600 to 3000 ppm essential oils. In some aspects, the collected distillate comprises from 2700 to 3000 ppm essential oils. In some aspects, the collected distillate comprises from 2800 to 3000 ppm essential oils. In some aspects, the collected distillate comprises from 2900 to 3000 ppm essential oils.

In some aspects, the collected distillate comprises 200, or 300, or 400, or 500, or 600, or 700, or 800, or 900, or 1000, or 1100, or 1200, or 1300, or 1400, or 1500, or 1600, or 1700, or 1800, or 1900, or 2000, or 2100, or 2200, or 2300, or 2400, or 2500, or 2600, or 2700, or 2800, or 2900, or 3000 ppm essential oils.

In some aspects, the collected distillate is further processed, thereby resulting in a composition comprising from 200 to 3000 ppm essential oils. In some aspects, the composition comprises from 200 to 2900 ppm essential oils. In some aspects, the composition comprises from 200 to 2800 ppm essential oils. In some aspects, the composition comprises from 200 to 2700 ppm essential oils. In some aspects, the composition comprises from 200 to 2600 ppm essential oils. In some aspects, the composition comprises from 200 to 2500 ppm essential oils. In some aspects, the composition comprises from 200 to 2400 ppm essential oils. In some aspects, the composition comprises from 200 to 2300 ppm essential oils. In some aspects, the composition comprises from 200 to 2200 ppm essential oils. In some aspects, the composition comprises from 200 to 2100 ppm essential oils. In some aspects, the composition comprises from 200 to 2000 ppm essential oils. In some aspects, the composition comprises from 200 to 1900 ppm essential oils. In some aspects, the composition comprises from 200 to 1800 ppm essential oils. In some aspects, the composition comprises from 200 to 1700 ppm essential oils. In some aspects, the composition comprises from 200 to 1600 ppm essential oils. In some aspects, the composition comprises from 200 to 1500 ppm essential oils. In some aspects, the composition comprises from 200 to 1400 ppm essential oils. In some aspects, the composition comprises from 200 to 1300 ppm essential oils. In some aspects, the composition comprises from 200 to 1200 ppm essential oils. In some aspects, the composition comprises from 200 to 1100 ppm essential oils. In some aspects, the composition comprises from 200 to 1000 ppm essential oils. In some aspects, the composition comprises from 200 to 900 ppm essential oils. In some aspects, the composition comprises from 200 to 800 ppm essential oils. In some aspects, the composition comprises from 200 to 700 ppm essential oils. In some aspects, the composition comprises from 200 to 600 ppm essential oils. In some aspects, the composition comprises from 200 to 500 ppm essential oils. In some aspects, the composition comprises from 200 to 400 ppm essential oils. In some aspects, the composition comprises from 200 to 300 ppm essential oils.

In some aspects, the composition comprises from 300 to 3000 ppm essential oils. In some aspects, the composition comprises from 400 to 3000 ppm essential oils. In some aspects, the composition comprises from 500 to 3000 ppm essential oils. In some aspects, the composition comprises from 600 to 3000 ppm essential oils. In some aspects, the composition comprises from 700 to 3000 ppm essential oils. In some aspects, the composition comprises from 800 to 3000 ppm essential oils. In some aspects, the composition comprises from 900 to 3000 ppm essential oils. In some aspects, the composition comprises from 1000 to 3000 ppm essential oils. In some aspects, the composition comprises from 1100 to 3000 ppm essential oils. In some aspects, the composition comprises from 1200 to 3000 ppm essential oils. In some aspects, the composition comprises from 1300 to 3000 ppm essential oils. In some aspects, the composition comprises from 1400 to 3000 ppm essential oils. In some aspects, the composition comprises from 1500 to 3000 ppm essential oils. In some aspects, the composition comprises from 1600 to 3000 ppm essential oils. In some aspects, the composition comprises from 1700 to 3000 ppm essential oils. In some aspects, the composition comprises from 1800 to 3000 ppm essential oils. In some aspects, the composition comprises from 1900 to 3000 ppm essential oils. In some aspects, the composition comprises from 2000 to 3000 ppm essential oils. In some aspects, the composition comprises from 2100 to 3000 ppm essential oils. In some aspects, the composition comprises from 2200 to 3000 ppm essential oils. In some aspects, the composition comprises from 2300 to 3000 ppm essential oils. In some aspects, the composition comprises from 2400 to 3000 ppm essential oils. In some aspects, the composition comprises from 2500 to 3000 ppm essential oils. In some aspects, the composition comprises from 2600 to 3000 ppm essential oils. In some aspects, the composition comprises from 2700 to 3000 ppm essential oils. In some aspects, the composition comprises from 2800 to 3000 ppm essential oils. In some aspects, the composition comprises from 2900 to 3000 ppm essential oils.

In some aspects, the composition comprises 200, or 300, or 400, or 500, or 600, or 700, or 800, or 900, or 1000, or 1100, or 1200, or 1300, or 1400, or 1500, or 1600, or 1700, or 1800, or 1900, or 2000, or 2100, or 2200, or 2300, or 2400, or 2500, or 2600, or 2700, or 2800, or 2900, or 3000 ppm essential oils.

### The Plant Biomass According to Some Aspects Presented Herein

As used herein, the term “biomass” refers to organic material isolated, derived, or obtained from plants. Plant biomass may be isolated, derived, or obtained from any part of the plant, such as, for example, the leaves, the flowers, the stems, the roots, the fruit, the entire plant, the seeds, or any combination thereof. In particular the fruit can be strawberry.

The plant biomass may be freshly obtained, or, alternatively, the plant biomass may be dried. As used herein, the term “freshly obtained” refers to plant biomass that retains all, or greater than 90% of its intrinsic water.

In some aspects, the plant biomass retains from 50 to 90% of its intrinsic water. In some aspects, the plant biomass retains from 55 to 90% of its intrinsic water. In some aspects, the plant biomass retains from 60 to 90% of its intrinsic water. In some aspects, the plant biomass retains from 65 to 90% of its intrinsic water. In some aspects, the plant biomass retains from 70 to 90% of its intrinsic water. In some aspects, the plant biomass retains from 75 to 90% of its intrinsic water. In some aspects, the plant biomass retains from 80 to 90% of its intrinsic water. In some aspects, the plant biomass retains from 85 to 90% of its intrinsic water.

In some aspects, the plant biomass retains from 50 to 85% of its intrinsic water. In some aspects, the plant biomass retains from 50 to 80% of its intrinsic water. In some aspects, the plant biomass retains from 50 to 75% of its intrinsic water. In some aspects, the plant biomass retains from 50 to 70% of its intrinsic water. In some aspects, the plant biomass retains from 50 to 65% of its intrinsic water. In some aspects, the plant biomass retains from 50 to 60% of its intrinsic water. In some aspects, the plant biomass retains from 50 to 55% of its intrinsic water.

In some aspects, the plant biomass retains 50, or 55, or 60, or 65, or 70, or 75, or 80, or 85, or 90, or 95% of its intrinsic water, or greater.

In some aspects, the plant biomass may be processed prior to treatment according to the methods presented herein. Examples of processing include, but are not limited to, cutting, chopping, grinding, crushing, wetting, and the like.

In some aspects, the water is added to the plant biomass to raise the moisture content to 90% or greater. In some aspects, the moisture content of the plant biomass after water has been added is from 50 to 90%. In some aspects, the moisture content of the plant biomass after water has been added is from 55 to 90%. In some aspects, the moisture content of the plant biomass after water has been added is from 60 to 90%. In some aspects, the moisture content of the plant biomass after water has been added is from 65 to 90%. In some aspects, the moisture content of the plant biomass after water has been added is from 70 to 90%. In some aspects, the moisture content of the plant biomass after water has been added is from 75 to 90%. In some aspects, the moisture content of the plant biomass after water has been added is from 80 to 90%. In some aspects, the moisture content of the plant biomass after water has been added is from 85 to 90%.

In some aspects, the moisture content of the plant biomass after water has been added is from 50 to 90%. In some aspects, the moisture content of the plant biomass after water has been added is from 50 to 85%. In some aspects, the moisture content of the plant biomass after water has been added is from 50 to 80%. In some aspects, the moisture content of the plant biomass after water has been added is from 50 to 75%. In some aspects, the moisture content of the plant biomass after water has been added is from 50 to 70%. In some aspects, the moisture content of the plant biomass after water has been added is from 50 to 65%. In some aspects, the moisture content of the plant biomass after water has been added is from 50 to 60%. In some aspects, the moisture content of the plant biomass after water has been added is from 50 to 55%.

In some aspects, the moisture content of the plant biomass after water has been added is 50, or 55, or 60, or 65, or 70, or 75, or 80, or 85, or 90, or 95%.

In some aspects, the plant biomass comprises freshly obtained flowers. In some aspects, the flowers are selected from the group consisting of: flowers from a plant of the genus Oleaceae, flowers from Polianthes tuberosa, flowers from a plant of the genus Rosa, flowers from Lilac plants, Flowers from Lily of the Valley plants, Flowers from Mock Orange plants, flowers from Honeysuckle plants, flowers from Cananga odorata, flowers from Artabotrys odoratissimus, and flowers from Wisteria plants.

In some aspects, the plant of the genus Oleaceae is a plant of the Jasminium species is selected from the group consisting of Jasminium grandiflorum and Jasminium sambac.

In some aspects, the plant biomass comprises freshly obtained fruit. In some aspects, the fruit comprises fruit from the genus Pyrus. In some aspects, the plant biomass comprises dried fruit, such as dried fruit from Piper nigrum. In some aspects, the plant biomass comprises roots, such as, for example the roots of Zingiber officinale. In some aspects, the plant biomass comprises leaves, such as, for example leaves from Camellia sinensis.

Referring to Examples 1 and 2 below, by way of illustration only, the methods and apparatus presented herein do not require the addition of solvents, and produce essential oils having improved olfactive profiles. In some aspects, the improved olfactive profile may be an olfactive profile the same, or highly similar to the fresh plant. Alternatively, the improved olfactive profile may be an olfactive profile having fewer artefacts, such as, for example, undesired olfactive notes.

### The Extracts According to Some Aspects Presented Herein

Referring to Examples 1 and 2 below, by way of illustration only, the methods and apparatus presented herein do not require the addition of solvents, and produce essential oils having improved olfactive profiles. In some aspects, the improved olfactive profile may be an olfactive profile the same, or highly similar to the fresh plant. Alternatively, the improved olfactive profile may be an olfactive profile having fewer artefacts, such as, for example, undesired olfactive notes.

While Examples 1 and 2 depict the use of the methods and apparatus presented herein using freshly isolated flowers from Jasminium grandiflorum, one of ordinary skill in the art may readily appreciate the application of the same methods and/or apparatus to other sources of plant biomass.

In some aspects, the improved olfactive profile may be an olfactive profile the same, or highly similar to the fresh plant. In some aspects, the improved olfactive profile may be a result of changes in the relative proportions of one, or more than one compound within the essential oil. By way of illustration, the essential oil described in Example 2 comprises at least 5% w/w (±)-3,7-Dimethyl-1,6-octadien-3-ol, at least 7% w/w (z)-jasmone, and at least 20% w/w benzyl acetate, wherein the levels of these compounds are higher compared to essential oils derived from Jasminium grandiflorum, using conventional methods.

In some aspects, the improved olfactive profile may be an olfactive profile having fewer artefacts, such as, for example, undesired olfactive notes. By way of illustration, the essential oil described in Example 2 comprises no more than 6% w/w benzyl benzoate, wherein the level of the compound is lower compared to essential oils derived from Jasminium grandiflorum, using conventional methods.

The olfactive profile of the essential oil may be determined by any method readily selected by one of ordinary skill in the art, such as, for example, sensory testing, chemical analysis, and the like.

Essential Oil Derived from Jasminium grandiflorum: One aspect presented herein provides an essential oil extracted from at least one plant of the Jasminium species, wherein the essential oil comprises at least 7% w/w (z)-jasmone.

In some aspects, the at least one plant of the Jasminium species is selected from the group consisting of Jasminium grandiflorum and Jasminium sambac.

Without intending to be limited to any particular theory, (z)-jasmone is a molecule that contributes to the jasmine flower scent, wherein essential oils having higher levels of (z)-jasmone would smell more like fresh jasmine flowers, compared to essential oils having lower levels of (z)-jasmone.

In some aspects, the essential oil comprises from 7 to 15% w/w (z)-jasmone, which is higher compared to essential oils derived from Jasminium grandiflorum, using conventional methods. Accordingly, the essential oil smells more like fresh jasmine flowers, compared to essential oils having lower levels of (z)-jasmone.

In some aspects, the essential oil comprises from 7 to 15% w/w (z)-jasmone. In some aspects, the essential oil comprises from 7 to 14% w/w (z)-jasmone. In some aspects, the essential oil comprises from 7 to 13% w/w (z)-jasmone. In some aspects, the essential oil comprises from 7 to 12% w/w (z)-jasmone. In some aspects, the essential oil comprises from 7 to 11% w/w (z)-jasmone. In some aspects, the essential oil comprises from 7 to 10% w/w (z)-jasmone. In some aspects, the essential oil comprises from 7 to 9% w/w (z)-jasmone. In some aspects, the essential oil comprises from 7 to 8% w/w (z)-jasmone.

In some aspects, the essential oil comprises from 8 to 15% w/w (z)-jasmone. In some aspects, the essential oil comprises from 9 to 15% w/w (z)-jasmone. In some aspects, the essential oil comprises from 10 to 15% w/w (z)-jasmone. In some aspects, the essential oil comprises from 11 to 15% w/w (z)-jasmone. In some aspects, the essential oil comprises from 12 to 15% w/w (z)-jasmone. In some aspects, the essential oil comprises from 13 to 15% w/w (z)-jasmone. In some aspects, the essential oil comprises from 14 to 15% w/w (z)-jasmone.

In some aspects, the essential oil comprises 7, or 8, or 9, or 10, or 11, or 12, or 13, or 14, or 15% w/w (z)-jasmone.

Other molecules that may contribute to the jasmine flower scent include (±)-3,7-Dimethyl-1,6-octadien-3-ol and benzyl acetate. In some aspects, the levels of either (±)-3,7-Dimethyl-1,6-octadien-3-ol and benzyl acetate, or both are higher compared to essential oils derived from Jasminium grandiflorum, using conventional methods. Accordingly, the essential oil smells more like fresh jasmine flowers, compared to essential oils having lower levels of (±)-3,7-Dimethyl-1,6-octadien-3-ol and benzyl acetate, w benzyl acetate, or both.

In some aspects, the essential oil further comprises from 10 to 20% w/w (±)-3,7-Dimethyl-1,6-octadien-3-ol. In some aspects, the essential oil further comprises from 10 to 19% w/w (±)-3,7-Dimethyl-1,6-octadien-3-ol. In some aspects, the essential oil further comprises from 10 to 18% w/w (±)-3,7-Dimethyl-1,6-octadien-3-ol. In some aspects, the essential oil further comprises from 10 to 17% w/w (±)-3,7-Dimethyl-1,6-octadien-3-ol. In some aspects, the essential oil further comprises from 10 to 16% w/w (±)-3,7-Dimethyl-1,6-octadien-3-ol. In some aspects, the essential oil further comprises from 10 to 15% w/w (±)-3,7-Dimethyl-1,6-octadien-3-ol. In some aspects, the essential oil further comprises from 10 to 14% w/w (±)-3,7-Dimethyl-1,6-octadien-3-ol. In some aspects, the essential oil further comprises from 10 to 13% w/w (±)-3,7-Dimethyl-1,6-octadien-3-ol. In some aspects, the essential oil further comprises from 10 to 12% w/w (±)-3,7-Dimethyl-1,6-octadien-3-ol. In some aspects, the essential oil further comprises from 10 to 11% w/w (±)-3,7-Dimethyl-1,6-octadien-3-ol.

In some aspects, the essential oil further comprises from 11 to 20% w/w (±)-3,7-Dimethyl-1,6-octadien-3-ol. In some aspects, the essential oil further comprises from 12 to 20% w/w (±)-3,7-Dimethyl-1,6-octadien-3-ol. In some aspects, the essential oil further comprises from 13 to 20% w/w (±)-3,7-Dimethyl-1,6-octadien-3-ol. In some aspects, the essential oil further comprises from 14 to 20% w/w (±)-3,7-Dimethyl-1,6-octadien-3-ol. In some aspects, the essential oil further comprises from 15 to 20% w/w (±)-3,7-Dimethyl-1,6-octadien-3-ol. In some aspects, the essential oil further comprises from 16 to 20% w/w (±)-3,7-Dimethyl-1,6-octadien-3-ol. In some aspects, the essential oil further comprises from 17 to 20% w/w (±)-3,7-Dimethyl-1,6-octadien-3-ol. In some aspects, the essential oil further comprises from 18 to 20% w/w (±)-3,7-Dimethyl-1,6-octadien-3-ol. In some aspects, the essential oil further comprises from 19 to 20% w/w (±)-3,7-Dimethyl-1,6-octadien-3-ol.

In some aspects, the essential oil further comprises at least 5% w/w (±)-3,7-Dimethyl-1,6-octadien-3-ol.

In some aspects, the essential oil further comprises at least 16% w/w (±)-3,7-Dimethyl-1,6-octadien-3-ol.

In some aspects, the essential oil further comprises 10, or 11, or 12, or 13, or 14, or 15, or 16, or 17, or 18, or 19, or 20% w/w (±)-3,7-Dimethyl-1,6-octadien-3-ol.

In some aspects, the essential oil further comprises from 20 to 70% w/w benzyl acetate. In some aspects, the essential oil further comprises from 20 to 65% w/w benzyl acetate. In some aspects, the essential oil further comprises from 20 to 60% w/w benzyl acetate. In some aspects, the essential oil further comprises from 20 to 55% w/w benzyl acetate. In some aspects, the essential oil further comprises from 20 to 50% w/w benzyl acetate. In some aspects, the essential oil further comprises from 20 to 45% w/w benzyl acetate. In some aspects, the essential oil further comprises from 20 to 40% w/w benzyl acetate. In some aspects, the essential oil further comprises from 20 to 35% w/w benzyl acetate. In some aspects, the essential oil further comprises from 20 to 30% w/w benzyl acetate. In some aspects, the essential oil further comprises from 20 to 25% w/w benzyl acetate.

In some aspects, the essential oil further comprises from 25 to 70% w/w benzyl acetate. In some aspects, the essential oil further comprises from 30 to 70% w/w benzyl acetate. In some aspects, the essential oil further comprises from 35 to 70% w/w benzyl acetate. In some aspects, the essential oil further comprises from 40 to 70% w/w benzyl acetate. In some aspects, the essential oil further comprises from 45 to 70% w/w benzyl acetate. In some aspects, the essential oil further comprises from 50 to 70% w/w benzyl acetate. In some aspects, the essential oil further comprises from 55 to 70% w/w benzyl acetate. In some aspects, the essential oil further comprises from 60 to 70% w/w benzyl acetate. In some aspects, the essential oil further comprises from 65 to 70% w/w benzyl acetate.

In some aspects, the essential oil further comprises from 40 to 65% w/w benzyl acetate. In some aspects, the essential oil further comprises from 40 to 64% w/w benzyl acetate. In some aspects, the essential oil further comprises from 40 to 63% w/w benzyl acetate. In some aspects, the essential oil further comprises from 40 to 62% w/w benzyl acetate. In some aspects, the essential oil further comprises from 40 to 61% w/w benzyl acetate. In some aspects, the essential oil further comprises from 40 to 60% w/w benzyl acetate. In some aspects, the essential oil further comprises from 40 to 59% w/w benzyl acetate. In some aspects, the essential oil further comprises from 40 to 58% w/w benzyl acetate. In some aspects, the essential oil further comprises from 40 to 57% w/w benzyl acetate. In some aspects, the essential oil further comprises from 40 to 56% w/w benzyl acetate. In some aspects, the essential oil further comprises from 40 to 55% w/w benzyl acetate. In some aspects, the essential oil further comprises from 40 to 54% w/w benzyl acetate. In some aspects, the essential oil further comprises from 40 to 53% w/w benzyl acetate. In some aspects, the essential oil further comprises from 40 to 52% w/w benzyl acetate. In some aspects, the essential oil further comprises from 40 to 51% w/w benzyl acetate. In some aspects, the essential oil further comprises from 40 to 50% w/w benzyl acetate. In some aspects, the essential oil further comprises from 40 to 49% w/w benzyl acetate. In some aspects, the essential oil further comprises from 40 to 48% w/w benzyl acetate. In some aspects, the essential oil further comprises from 40 to 47% w/w benzyl acetate. In some aspects, the essential oil further comprises from 40 to 46% w/w benzyl acetate. In some aspects, the essential oil further comprises from 40 to 45% w/w benzyl acetate. In some aspects, the essential oil further comprises from 40 to 44% w/w benzyl acetate. In some aspects, the essential oil further comprises from 40 to 43% w/w benzyl acetate. In some aspects, the essential oil further comprises from 40 to 42% w/w benzyl acetate. In some aspects, the essential oil further comprises from 40 to 41% w/w benzyl acetate.

In some aspects, the essential oil further comprises from 41 to 65% w/w benzyl acetate. In some aspects, the essential oil further comprises from 42 to 65% w/w benzyl acetate. In some aspects, the essential oil further comprises from 43 to 65% w/w benzyl acetate. In some aspects, the essential oil further comprises from 44 to 65% w/w benzyl acetate. In some aspects, the essential oil further comprises from 45 to 65% w/w benzyl acetate. In some aspects, the essential oil further comprises from 46 to 65% w/w benzyl acetate. In some aspects, the essential oil further comprises from 47 to 65% w/w benzyl acetate. In some aspects, the essential oil further comprises from 48 to 65% w/w benzyl acetate. In some aspects, the essential oil further comprises from 49 to 65% w/w benzyl acetate. In some aspects, the essential oil further comprises from 50 to 65% w/w benzyl acetate. In some aspects, the essential oil further comprises from 51 to 65% w/w benzyl acetate. In some aspects, the essential oil further comprises from 52 to 65% w/w benzyl acetate. In some aspects, the essential oil further comprises from 53 to 65% w/w benzyl acetate. In some aspects, the essential oil further comprises from 54 to 65% w/w benzyl acetate. In some aspects, the essential oil further comprises from 55 to 65% w/w benzyl acetate. In some aspects, the essential oil further comprises from 56 to 65% w/w benzyl acetate. In some aspects, the essential oil further comprises from 57 to 65% w/w benzyl acetate. In some aspects, the essential oil further comprises from 58 to 65% w/w benzyl acetate. In some aspects, the essential oil further comprises from 59 to 65% w/w benzyl acetate. In some aspects, the essential oil further comprises from 60 to 65% w/w benzyl acetate. In some aspects, the essential oil further comprises from 61 to 65% w/w benzyl acetate. In some aspects, the essential oil further comprises from 62 to 65% w/w benzyl acetate. In some aspects, the essential oil further comprises from 63 to 65% w/w benzyl acetate. In some aspects, the essential oil further comprises from 64 to 65% w/w benzyl acetate.

In some aspects, the essential oil further comprises at least 20% w/w benzyl acetate.

In some aspects, the essential oil further comprises 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, or 61, or 62, or 63, or 64, or 65 /w benzyl acetate.

Molecules that may introduce an artifact to the jasmine flower scent include benzyl benzoate. In some aspects, the levels benzyl benzoate are lower compared to essential oils derived from Jasminium grandiflorum, using conventional methods. Accordingly, the essential oil smells more like fresh jasmine flowers, compared to essential oils having higher levels of benzyl benzoate.

In some aspects, the essential oil further comprises no more than 7% w/w benzyl benzoate.

In some aspects, the essential oil further comprises from 1 to 6.5% w/w benzyl benzoate. In some aspects, the essential oil further comprises from 1 to 6.4% w/w benzyl benzoate. In some aspects, the essential oil further comprises from 1 to 6.3% w/w benzyl benzoate. In some aspects, the essential oil further comprises from 1 to 6.2% w/w benzyl benzoate. In some aspects, the essential oil further comprises from 1 to 6.1% w/w benzyl benzoate. In some aspects, the essential oil further comprises from 1 to 6% w/w benzyl benzoate. In some aspects, the essential oil further comprises from 1 to 5.9% w/w benzyl benzoate. In some aspects, the essential oil further comprises from 1 to 5.8% w/w benzyl benzoate. In some aspects, the essential oil further comprises from 1 to 5.7% w/w benzyl benzoate. In some aspects, the essential oil further comprises from 1 to 5.6% w/w benzyl benzoate. In some aspects, the essential oil further comprises from 1 to 5.5% w/w benzyl benzoate. In some aspects, the essential oil further comprises from 1 to 5.4% w/w benzyl benzoate. In some aspects, the essential oil further comprises from 1 to 5.3% w/w benzyl benzoate. In some aspects, the essential oil further comprises from 1 to 5.2% w/w benzyl benzoate. In some aspects, the essential oil further comprises from 1 to 5.1% w/w benzyl benzoate. In some aspects, the essential oil further comprises from 1 to 5% w/w benzyl benzoate. In some aspects, the essential oil further comprises from 1 to 4.9% w/w benzyl benzoate. In some aspects, the essential oil further comprises from 1 to 4.8% w/w benzyl benzoate. In some aspects, the essential oil further comprises from 1 to 4.7% w/w benzyl benzoate. In some aspects, the essential oil further comprises from 1 to 4.6% w/w benzyl benzoate. In some aspects, the essential oil further comprises from 1 to 4.5% w/w benzyl benzoate. In some aspects, the essential oil further comprises from 1 to 4.4% w/w benzyl benzoate. In some aspects, the essential oil further comprises from 1 to 4.3% w/w benzyl benzoate. In some aspects, the essential oil further comprises from 1 to 4.2% w/w benzyl benzoate. In some aspects, the essential oil further comprises from 1 to 4.1% w/w benzyl benzoate. In some aspects, the essential oil further comprises from 1 to 4% w/w benzyl benzoate. In some aspects, the essential oil further comprises from 1 to 3.9% w/w benzyl benzoate. In some aspects, the essential oil further comprises from 1 to 3.8% w/w benzyl benzoate. In some aspects, the essential oil further comprises from 1 to 3.7% w/w benzyl benzoate. In some aspects, the essential oil further comprises from 1 to 3.6% w/w benzyl benzoate. In some aspects, the essential oil further comprises from 1 to 3.5% w/w benzyl benzoate. In some aspects, the essential oil further comprises from 1 to 3.4% w/w benzyl benzoate. In some aspects, the essential oil further comprises from 1 to 3.3% w/w benzyl benzoate. In some aspects, the essential oil further comprises from 1 to 3.2% w/w benzyl benzoate. In some aspects, the essential oil further comprises from 1 to 3.1% w/w benzyl benzoate. In some aspects, the essential oil further comprises from 1 to 3% w/w benzyl benzoate. In some aspects, the essential oil further comprises from 1 to 2.9% w/w benzyl benzoate. In some aspects, the essential oil further comprises from 1 to 2.8% w/w benzyl benzoate. In some aspects, the essential oil further comprises from 1 to 2.7% w/w benzyl benzoate. In some aspects, the essential oil further comprises from 1 to 2.6% w/w benzyl benzoate. In some aspects, the essential oil further comprises from 1 to 2.5% w/w benzyl benzoate. In some aspects, the essential oil further comprises from 1 to 2.4% w/w benzyl benzoate. In some aspects, the essential oil further comprises from 1 to 2.3% w/w benzyl benzoate. In some aspects, the essential oil further comprises from 1 to 2.2% w/w benzyl benzoate. In some aspects, the essential oil further comprises from 1 to 2.1% w/w benzyl benzoate. In some aspects, the essential oil further comprises from 1 to 2% w/w benzyl benzoate. In some aspects, the essential oil further comprises from 1 to 1.9% w/w benzyl benzoate. In some aspects, the essential oil further comprises from 1 to 1.8% w/w benzyl benzoate. In some aspects, the essential oil further comprises from 1 to 1.7% w/w benzyl benzoate. In some aspects, the essential oil further comprises from 1 to 1.6% w/w benzyl benzoate. In some aspects, the essential oil further comprises from 1 to 1.5% w/w benzyl benzoate. In some aspects, the essential oil further comprises from 1 to 1.4% w/w benzyl benzoate. In some aspects, the essential oil further comprises from 1 to 1.3% w/w benzyl benzoate. In some aspects, the essential oil further comprises from 1 to 1.2% w/w benzyl benzoate. In some aspects, the essential oil further comprises from 1 to 1.1% w/w benzyl benzoate.

In some aspects, the essential oil further comprises from 1 to 6.5% w/w benzyl benzoate. In some aspects, the essential oil further comprises from 1.1 to 6.5% w/w benzyl benzoate. In some aspects, the essential oil further comprises from 1.2 to 6.5% w/w benzyl benzoate. In some aspects, the essential oil further comprises from 1.3 to 6.5% w/w benzyl benzoate. In some aspects, the essential oil further comprises from 1.4 to 6.5% w/w benzyl benzoate. In some aspects, the essential oil further comprises from 1.5 to 6.5% w/w benzyl benzoate. In some aspects, the essential oil further comprises from 1.6 to 6.5% w/w benzyl benzoate. In some aspects, the essential oil further comprises from 1.7 to 6.5% w/w benzyl benzoate. In some aspects, the essential oil further comprises from 1.8 to 6.5% w/w benzyl benzoate. In some aspects, the essential oil further comprises from 1.9 to 6.5% w/w benzyl benzoate. In some aspects, the essential oil further comprises from 2 to 6.5% w/w benzyl benzoate. In some aspects, the essential oil further comprises from 2.1 to 6.5% w/w benzyl benzoate. In some aspects, the essential oil further comprises from 2.2 to 6.5% w/w benzyl benzoate. In some aspects, the essential oil further comprises from 2.3 to 6.5% w/w benzyl benzoate. In some aspects, the essential oil further comprises from 2.4 to 6.5% w/w benzyl benzoate. In some aspects, the essential oil further comprises from 2.5 to 6.5% w/w benzyl benzoate. In some aspects, the essential oil further comprises from 2.6 to 6.5% w/w benzyl benzoate. In some aspects, the essential oil further comprises from 2.7 to 6.5% w/w benzyl benzoate. In some aspects, the essential oil further comprises from 2.8 to 6.5% w/w benzyl benzoate. In some aspects, the essential oil further comprises from 2.9 to 6.5% w/w benzyl benzoate. In some aspects, the essential oil further comprises from 3 to 6.5% w/w benzyl benzoate. In some aspects, the essential oil further comprises from 3.1 to 6.5% w/w benzyl benzoate. In some aspects, the essential oil further comprises from 3.2 to 6.5% w/w benzyl benzoate. In some aspects, the essential oil further comprises from 3.3 to 6.5% w/w benzyl benzoate. In some aspects, the essential oil further comprises from 3.4 to 6.5% w/w benzyl benzoate. In some aspects, the essential oil further comprises from 3.5 to 6.5% w/w benzyl benzoate. In some aspects, the essential oil further comprises from 3.6 to 6.5% w/w benzyl benzoate. In some aspects, the essential oil further comprises from 3.7 to 6.5% w/w benzyl benzoate. In some aspects, the essential oil further comprises from 3.8 to 6.5% w/w benzyl benzoate. In some aspects, the essential oil further comprises from 3.9 to 6.5% w/w benzyl benzoate. In some aspects, the essential oil further comprises from 4 to 6.5% w/w benzyl benzoate. In some aspects, the essential oil further comprises from 4.1 to 6.5% w/w benzyl benzoate. In some aspects, the essential oil further comprises from 4.2 to 6.5% w/w benzyl benzoate. In some aspects, the essential oil further comprises from 4.3 to 6.5% w/w benzyl benzoate. In some aspects, the essential oil further comprises from 4.4 to 6.5% w/w benzyl benzoate. In some aspects, the essential oil further comprises from 4.5 to 6.5% w/w benzyl benzoate. In some aspects, the essential oil further comprises from 4.6 to 6.5% w/w benzyl benzoate. In some aspects, the essential oil further comprises from 4.7 to 6.5% w/w benzyl benzoate. In some aspects, the essential oil further comprises from 4.8 to 6.5% w/w benzyl benzoate. In some aspects, the essential oil further comprises from 4.9 to 6.5% w/w benzyl benzoate. In some aspects, the essential oil further comprises from 5 to 6.5% w/w benzyl benzoate. In some aspects, the essential oil further comprises from 5.1 to 6.5% w/w benzyl benzoate. In some aspects, the essential oil further comprises from 5.2 to 6.5% w/w benzyl benzoate. In some aspects, the essential oil further comprises from 5.3 to 6.5% w/w benzyl benzoate. In some aspects, the essential oil further comprises from 5.4 to 6.5% w/w benzyl benzoate. In some aspects, the essential oil further comprises from 5.5 to 6.5% w/w benzyl benzoate. In some aspects, the essential oil further comprises from 5.6 to 6.5% w/w benzyl benzoate. In some aspects, the essential oil further comprises from 5.7 to 6.5% w/w benzyl benzoate. In some aspects, the essential oil further comprises from 5.8 to 6.5% w/w benzyl benzoate. In some aspects, the essential oil further comprises from 5.9 to 6.5% w/w benzyl benzoate. In some aspects, the essential oil further comprises from 6 to 6.5% w/w benzyl benzoate. In some aspects, the essential oil further comprises from 6.1 to 6.5% w/w benzyl benzoate. In some aspects, the essential oil further comprises from 6.2 to 6.5% w/w benzyl benzoate. In some aspects, the essential oil further comprises from 6.3 to 6.5% w/w benzyl benzoate. In some aspects, the essential oil further comprises from 6.4 to 6.5% w/w benzyl benzoate.

In some aspects, the essential oil further comprises 1, or 1.1, or 1.2, or 1.3, or 1.4, or 1.5, or 1.6, or 1.7, or 1.8, or 1.9, or 2, or 2.1, or 2.2, or 2.3, or 2.4, or 2.5, or 2.6, or 2.7, or 2.8, or 2.9, or 3, or 3.1, or 3.2, or 3.3, or 3.4, or 3.5, or 3.6, or 3.7, or 3.8, or 3.9, or 4, or 4.1, or 4.2, or 4.3, or 4.4, or 4.5, or 4.6, or 4.7, or 4.8, or 4.9, or 5, or 5.1, or 5.2, or 5.3, or 5.4, or 5.5, or 5.6, or 5.7, or 5.8, or 5.9, or 6, or 6.1, or 6.2, or 6.3, or 6.4, or 6.5% w/w benzyl benzoate.

In some aspects, the essential oil comprises from 7 to 11% w/w (z)-jasmone, from 10 to 17% w/w (±)-3,7-Dimethyl-1,6-octadien-3-ol, from 40 to 62% w/w benzyl acetate, and from 1 to 7.1% w/w benzyl benzoate.

In some aspects, the essential oil comprises any one of Microwave Extract 1, Microwave Extract 2, or Microwave Extract 3, disclosed in Table 1.

In some aspects, the essential oil comprises the compounds, at the amounts disclosed in any one of Microwave Extract 1, Microwave Extract 2, or Microwave Extract 3, disclosed in Table 1.

Essential Oil Derived from Piper nigrum: One aspect presented herein provides an essential oil extracted from Piper nigrum, wherein the essential oil comprises at least 7% w/w sabinine.

In some aspects, the essential oil comprises from 9 to 15% w/w sabinine. In some aspects, the essential oil comprises from 9 to 15% w/w sabinine. In some aspects, the essential oil comprises from 10 to 15% w/w sabinine. In some aspects, the essential oil comprises from 11 to 15% w/w sabinine. In some aspects, the essential oil comprises from 12 to 15% w/w sabinine. In some aspects, the essential oil comprises from 13 to 15% w/w sabinine. In some aspects, the essential oil comprises from 14 to 15% w/w sabinine.

In some aspects, the essential oil comprises from 9 to 14% w/w sabinine. In some aspects, the essential oil comprises from 9 to 13% w/w sabinine. In some aspects, the essential oil comprises from 9 to 12% w/w sabinine. In some aspects, the essential oil comprises from 9 to 11% w/w sabinine. In some aspects, the essential oil comprises from 9 to 10% w/w sabinine.

In some aspects, the essential oil comprises 9, or 10, or 11, or 12, or 13, or 14, or 15% w/w sabinine.

In some aspects, the essential oil further comprises from 12 to 16% w/w β-pinene. In some aspects, the essential oil further comprises from 13 to 16% w/w β-pinene. In some aspects, the essential oil further comprises from 14 to 16% w/w β-pinene. In some aspects, the essential oil further comprises from 15 to 16% w/w β-pinene.

In some aspects, the essential oil further comprises from 12 to 15% w/w β-pinene. In some aspects, the essential oil further comprises from 12 to 14% w/w β-pinene. In some aspects, the essential oil further comprises from 12 to 13% w/w β-pinene.

In some aspects, the essential oil further comprises 12, or 13, or 14, or 15, or 16% w/w β-pinene.

In some aspects, the essential oil further comprises from 20 to 30% w/w limonene. In some aspects, the essential oil further comprises from 21 to 30% w/w limonene. In some aspects, the essential oil further comprises from 22 to 30% w/w limonene. In some aspects, the essential oil further comprises from 23 to 30% w/w limonene. In some aspects, the essential oil further comprises from 24 to 30% w/w limonene. In some aspects, the essential oil further comprises from 25 to 30% w/w limonene. In some aspects, the essential oil further comprises from 26 to 30% w/w limonene. In some aspects, the essential oil further comprises from 27 to 30% w/w limonene. In some aspects, the essential oil further comprises from 28 to 30% w/w limonene. In some aspects, the essential oil further comprises from 29 to 30% w/w limonene.

In some aspects, the essential oil further comprises from 20 to 29% w/w limonene. In some aspects, the essential oil further comprises from 20 to 28% w/w limonene. In some aspects, the essential oil further comprises from 20 to 27% w/w limonene. In some aspects, the essential oil further comprises from 20 to 26% w/w limonene. In some aspects, the essential oil further comprises from 20 to 25% w/w limonene. In some aspects, the essential oil further comprises from 20 to 24% w/w limonene. In some aspects, the essential oil further comprises from 20 to 23% w/w limonene. In some aspects, the essential oil further comprises from 20 to 22% w/w limonene. In some aspects, the essential oil further comprises from 20 to 21% w/w limonene.

In some aspects, the essential oil further comprises 20, or 21, or 22, or 23, or 24, or 25, or 26, or 27, or 28, or 29, or 30% w/w limonene.

In some aspects, the essential oil further comprises from 7 to 12% w/w beta-caryophyllene. In some aspects, the essential oil further comprises from 8 to 12% w/w beta-caryophyllene. In some aspects, the essential oil further comprises from 9 to 12% w/w beta-caryophyllene. In some aspects, the essential oil further comprises from 10 to 12% w/w beta-caryophyllene. In some aspects, the essential oil further comprises from 11 to 12% w/w beta-caryophyllene.

In some aspects, the essential oil further comprises from 7 to 11% w/w beta-caryophyllene. In some aspects, the essential oil further comprises from 7 to 10% w/w beta-caryophyllene. In some aspects, the essential oil further comprises from 7 to 9% w/w beta-caryophyllene. In some aspects, the essential oil further comprises from 7 to 8% w/w beta-caryophyllene.

In some aspects, the essential oil further comprises 7, or 8, or 9, or 10, or 11, or 12% w/w beta-caryophyllene.

In some aspects, the essential oil further comprises less than 3% w/w piperine.

In some aspects, the essential oil comprises the compounds, at the amounts disclosed in any one of Microwave Extract 1, or Microwave Extract 2 disclosed in Table 2.

Referring to Table 2, and without intending to be limited to any particular theory, the olfactive properties of the essential oil derived from Piper nigrum according to the methods described herein differ from essential oils derived from Piper nigrum according to conventional SFE or steam distillation methods: The essential oil derived from Piper nigrum according to the methods described herein was less woody (possibly due, in part to the lower levels of of β-caryophyllene, α-humulene and germacrene in the essential oil derived from Piper nigrum according to the methods described herein, compared to essential oils derived from Piper nigrum according to conventional SFE or steam distillation methods). Further, a major terpenic and “paint” note was noted in the essential oil derived from Piper nigrum according to the methods described herein, compared to essential oils derived from Piper nigrum according to conventional SFE or steam distillation methods, possibly due to a higher amount of β-pinene, and/or myrcene in the essential oil derived from Piper nigrum according to the methods described herein, compared to essential oils derived from Piper nigrum according to conventional SFE or steam distillation methods. Additionally, a fresher note was reported in the essential oil derived from Piper nigrum according to the methods described herein, compared to essential oils derived from Piper nigrum according to conventional SFE or steam distillation methods, possibly due to a higher amount of limonene in in the essential oil derived from Piper nigrum according to the methods described herein, compared to essential oils derived from Piper nigrum according to conventional SFE or steam distillation methods.

In addition, essential oils derived from Piper nigrum according to conventional SFE or steam distillation methods were reported to have stronger woody, piney, resinous, dirty, floral, and moldy notes, possibly due to the higher amounts of α-copaene, α-phellandrene, β-bisabolene, α- and β-pinene, d3-carene, α-phellandrene, α-terpinene, β-cadinene, gamma-terpinene, terpinolene, terpinen-4-ol, and linalool, compared to the essential oil derived from Piper nigrum according to the methods described herein.

Essential Oil Derived from Ginger Root: One aspect presented herein provides an essential oil extracted from ginger root, wherein the essential oil comprises at least 13% w/w eucalyptol.

In some aspects, the essential oil comprises from 13 to 15% w/w eucalyptol. In some aspects, the essential oil comprises from 13 to 14% w/w eucalyptol.

In some aspects, the essential oil comprises 13, or 14, or 15% w/w eucalyptol.

In some aspects, the essential oil comprises from 2 to 6% w/w 6-methyl-5-hepten-2-one. In some aspects, the essential oil comprises from 3 to 6% w/w 6-methyl-5-hepten-2-one. In some aspects, the essential oil comprises from 4 to 6% w/w 6-methyl-5-hepten-2-one. In some aspects, the essential oil comprises from 5 to 6% w/w 6-methyl-5-hepten-2-one.

In some aspects, the essential oil comprises from 2 to 5% w/w 6-methyl-5-hepten-2-one. In some aspects, the essential oil comprises from 2 to 4% w/w 6-methyl-5-hepten-2-one. In some aspects, the essential oil comprises from 2 to 3% w/w 6-methyl-5-hepten-2-one.

In some aspects, the essential oil comprises 2, or 3, or 4, or 5, or 6% w/w 6-methyl-5-hepten-2-one.

In some aspects, the essential oil comprises from 19 to 25% w/w neral. In some aspects, the essential oil comprises from 20 to 25% w/w neral. In some aspects, the essential oil comprises from 21 to 25% w/w neral. In some aspects, the essential oil comprises from 22 to 25% w/w neral. In some aspects, the essential oil comprises from 23 to 25% w/w neral. In some aspects, the essential oil comprises from 24 to 25% w/w neral.

In some aspects, the essential oil comprises from 19 to 24% w/w neral. In some aspects, the essential oil comprises from 19 to 23% w/w neral. In some aspects, the essential oil comprises from 19 to 22% w/w neral. In some aspects, the essential oil comprises from 19 to 21% w/w neral. In some aspects, the essential oil comprises from 19 to 20% w/w neral.

In some aspects, the essential oil comprises 19, or 20, or 21, or 22, or 23, or 24, or 25% w/w neral.

In some aspects, the essential oil comprises from 26 to 29% w/w geranial. In some aspects, the essential oil comprises from 27 to 29% w/w geranial. In some aspects, the essential oil comprises from 28 to 29% w/w geranial.

In some aspects, the essential oil comprises from 26 to 28% w/w geranial. In some aspects, the essential oil comprises from 26 to 27% w/w geranial.

In some aspects, the essential oil comprises 26, or 27, or 28, or 29% w/w geranial.

In some aspects, the essential oil comprises greater than 0.1% w/w 2-heptanone.

In some aspects, the essential oil comprises greater than 0.2% w/w nerol.

In some aspects, the essential oil comprises less than 1% w/w geraniol.

In some aspects, the essential oil comprises less than 18.5% w/w zingiberene.

In some aspects, the essential oil comprises the compounds, at the amounts disclosed in any one of Microwave Extract 1, or Microwave Extract 2 disclosed in Table 5.

Referring to Table 5, and without intending to be limited to any particular theory, the essential oils derived from ginger root according to the methods disclosed herein possessed a more fresh green ginger profile, compared to essential oils derived from ginger root according to conventional extraction methods, perhaps due, in part to increased levels of fresh notes, such as eucalyptol, and floral notes, such as linalol and citranellol. Moreover, citrus aand green notes were elevated in essential oils derived from ginger root according to the methods disclosed herein, compared to essential oils derived from ginger root according to conventional extraction methods.

Extract Derived from Pears: One aspect presented herein provides an extract from pears, wherein the extract comprises at least 0.1% w/w hexyl acetate.

In some aspects, the extract comprises from 0.1 to 6% w/w hexyl acetate. In some aspects, the extract comprises from 0.2 to 6% w/w hexyl acetate. In some aspects, the extract comprises from 0.3 to 6% w/w hexyl acetate. In some aspects, the extract comprises from 0.4 to 6% w/w hexyl acetate. In some aspects, the extract comprises from 0.5 to 6% w/w hexyl acetate. In some aspects, the extract comprises from 0.6 to 6% w/w hexyl acetate. In some aspects, the extract comprises from 0.7 to 6% w/w hexyl acetate. In some aspects, the extract comprises from 0.8 to 6% w/w hexyl acetate. In some aspects, the extract comprises from 0.9 to 6% w/w hexyl acetate. In some aspects, the extract comprises from 1 to 6% w/w hexyl acetate. In some aspects, the extract comprises from 1.1 to 6% w/w hexyl acetate. In some aspects, the extract comprises from 1.2 to 6% w/w hexyl acetate. In some aspects, the extract comprises from 1.3 to 6% w/w hexyl acetate. In some aspects, the extract comprises from 1.4 to 6% w/w hexyl acetate. In some aspects, the extract comprises from 1.5 to 6% w/w hexyl acetate. In some aspects, the extract comprises from 1.6 to 6% w/w hexyl acetate. In some aspects, the extract comprises from 1.7 to 6% w/w hexyl acetate. In some aspects, the extract comprises from 1.8 to 6% w/w hexyl acetate. In some aspects, the extract comprises from 1.9 to 6% w/w hexyl acetate. In some aspects, the extract comprises from 2 to 6% w/w hexyl acetate. In some aspects, the extract comprises from 2.1 to 6% w/w hexyl acetate. In some aspects, the extract comprises from 2.2 to 6% w/w hexyl acetate. In some aspects, the extract comprises from 2.3 to 6% w/w hexyl acetate. In some aspects, the extract comprises from 2.4 to 6% w/w hexyl acetate. In some aspects, the extract comprises from 2.5 to 6% w/w hexyl acetate. In some aspects, the extract comprises from 2.6 to 6% w/w hexyl acetate. In some aspects, the extract comprises from 2.7 to 6% w/w hexyl acetate. In some aspects, the extract comprises from 2.8 to 6% w/w hexyl acetate. In some aspects, the extract comprises from 2.9 to 6% w/w hexyl acetate. In some aspects, the extract comprises from 3 to 6% w/w hexyl acetate. In some aspects, the extract comprises from 3.1 to 6% w/w hexyl acetate. In some aspects, the extract comprises from 3.2 to 6% w/w hexyl acetate. In some aspects, the extract comprises from 3.3 to 6% w/w hexyl acetate. In some aspects, the extract comprises from 3.4 to 6% w/w hexyl acetate. In some aspects, the extract comprises from 3.5 to 6% w/w hexyl acetate. In some aspects, the extract comprises from 3.6 to 6% w/w hexyl acetate. In some aspects, the extract comprises from 3.7 to 6% w/w hexyl acetate. In some aspects, the extract comprises from 3.8 to 6% w/w hexyl acetate. In some aspects, the extract comprises from 3.9 to 6% w/w hexyl acetate. In some aspects, the extract comprises from 4 to 6% w/w hexyl acetate. In some aspects, the extract comprises from 4.1 to 6% w/w hexyl acetate. In some aspects, the extract comprises from 4.2 to 6% w/w hexyl acetate. In some aspects, the extract comprises from 4.3 to 6% w/w hexyl acetate. In some aspects, the extract comprises from 4.4 to 6% w/w hexyl acetate. In some aspects, the extract comprises from 4.5 to 6% w/w hexyl acetate. In some aspects, the extract comprises from 4.6 to 6% w/w hexyl acetate. In some aspects, the extract comprises from 4.7 to 6% w/w hexyl acetate. In some aspects, the extract comprises from 4.8 to 6% w/w hexyl acetate. In some aspects, the extract comprises from 4.9 to 6% w/w hexyl acetate. In some aspects, the extract comprises from 5 to 6% w/w hexyl acetate. In some aspects, the extract comprises from 5.1 to 6% w/w hexyl acetate. In some aspects, the extract comprises from 5.2 to 6% w/w hexyl acetate. In some aspects, the extract comprises from 5.3 to 6% w/w hexyl acetate. In some aspects, the extract comprises from 5.4 to 6% w/w hexyl acetate. In some aspects, the extract comprises from 5.5 to 6% w/w hexyl acetate. In some aspects, the extract comprises from 5.6 to 6% w/w hexyl acetate. In some aspects, the extract comprises from 5.7 to 6% w/w hexyl acetate. In some aspects, the extract comprises from 5.8 to 6% w/w hexyl acetate. In some aspects, the extract comprises from 5.9 to 6% w/w hexyl acetate.

In some aspects, the extract comprises from 0.1 to 5.9% w/w hexyl acetate. In some aspects, the extract comprises from 0.1 to 5.8% w/w hexyl acetate. In some aspects, the extract comprises from 0.1 to 5.7% w/w hexyl acetate. In some aspects, the extract comprises from 0.1 to 5.6% w/w hexyl acetate. In some aspects, the extract comprises from 0.1 to 5.5% w/w hexyl acetate. In some aspects, the extract comprises from 0.1 to 5.4% w/w hexyl acetate. In some aspects, the extract comprises from 0.1 to 5.3% w/w hexyl acetate. In some aspects, the extract comprises from 0.1 to 5.2% w/w hexyl acetate. In some aspects, the extract comprises from 0.1 to 5.1% w/w hexyl acetate. In some aspects, the extract comprises from 0.1 to 5% w/w hexyl acetate. In some aspects, the extract comprises from 0.1 to 4.9% w/w hexyl acetate. In some aspects, the extract comprises from 0.1 to 4.8% w/w hexyl acetate. In some aspects, the extract comprises from 0.1 to 4.7% w/w hexyl acetate. In some aspects, the extract comprises from 0.1 to 4.6% w/w hexyl acetate. In some aspects, the extract comprises from 0.1 to 4.5% w/w hexyl acetate. In some aspects, the extract comprises from 0.1 to 4.4% w/w hexyl acetate. In some aspects, the extract comprises from 0.1 to 4.3% w/w hexyl acetate. In some aspects, the extract comprises from 0.1 to 4.2% w/w hexyl acetate. In some aspects, the extract comprises from 0.1 to 4.1% w/w hexyl acetate. In some aspects, the extract comprises from 0.1 to 4% w/w hexyl acetate. In some aspects, the extract comprises from 0.1 to 3.9% w/w hexyl acetate. In some aspects, the extract comprises from 0.1 to 3.8% w/w hexyl acetate. In some aspects, the extract comprises from 0.1 to 3.7% w/w hexyl acetate. In some aspects, the extract comprises from 0.1 to 3.6% w/w hexyl acetate. In some aspects, the extract comprises from 0.1 to 3.5% w/w hexyl acetate. In some aspects, the extract comprises from 0.1 to 3.4% w/w hexyl acetate. In some aspects, the extract comprises from 0.1 to 3.3% w/w hexyl acetate. In some aspects, the extract comprises from 0.1 to 3.2% w/w hexyl acetate. In some aspects, the extract comprises from 0.1 to 3.1% w/w hexyl acetate. In some aspects, the extract comprises from 0.1 to 3% w/w hexyl acetate. In some aspects, the extract comprises from 0.1 to 2.9% w/w hexyl acetate. In some aspects, the extract comprises from 0.1 to 2.8% w/w hexyl acetate. In some aspects, the extract comprises from 0.1 to 2.7% w/w hexyl acetate. In some aspects, the extract comprises from 0.1 to 2.6% w/w hexyl acetate. In some aspects, the extract comprises from 0.1 to 2.5% w/w hexyl acetate. In some aspects, the extract comprises from 0.1 to 2.4% w/w hexyl acetate. In some aspects, the extract comprises from 0.1 to 2.3% w/w hexyl acetate. In some aspects, the extract comprises from 0.1 to 2.2% w/w hexyl acetate. In some aspects, the extract comprises from 0.1 to 2.1% w/w hexyl acetate. In some aspects, the extract comprises from 0.1 to 2% w/w hexyl acetate. In some aspects, the extract comprises from 0.1 to 1.9% w/w hexyl acetate. In some aspects, the extract comprises from 0.1 to 1.8% w/w hexyl acetate. In some aspects, the extract comprises from 0.1 to 1.7% w/w hexyl acetate. In some aspects, the extract comprises from 0.1 to 1.6% w/w hexyl acetate. In some aspects, the extract comprises from 0.1 to 1.5% w/w hexyl acetate. In some aspects, the extract comprises from 0.1 to 1.4% w/w hexyl acetate. In some aspects, the extract comprises from 0.1 to 1.3% w/w hexyl acetate. In some aspects, the extract comprises from 0.1 to 1.2% w/w hexyl acetate. In some aspects, the extract comprises from 0.1 to 1.1% w/w hexyl acetate. In some aspects, the extract comprises from 0.1 to 1% w/w hexyl acetate. In some aspects, the extract comprises from 0.1 to 0.9% w/w hexyl acetate. In some aspects, the extract comprises from 0.1 to 0.8% w/w hexyl acetate. In some aspects, the extract comprises from 0.1 to 0.7% w/w hexyl acetate. In some aspects, the extract comprises from 0.1 to 0.6% w/w hexyl acetate. In some aspects, the extract comprises from 0.1 to 0.5% w/w hexyl acetate. In some aspects, the extract comprises from 0.1 to 0.4% w/w hexyl acetate. In some aspects, the extract comprises from 0.1 to 0.3% w/w hexyl acetate. In some aspects, the extract comprises from 0.1 to 0.2% w/w hexyl acetate.

In some aspects, the extract comprises 0.1, or 0.2, or 0.3, or 0.4, or 0.5, or 0.6, or 0.7, or 0.8, or 0.9, or 1, or 1.1, or 1.2, or 1.3, or 1.4, or 1.5, or 1.6, or 1.7, or 1.8, or 1.9, or 2, or 2.1, or 2.2, or 2.3, or 2.4, or 2.5, or 2.6, or 2.7, or 2.8, or 2.9, or 3, or 3.1, or 3.2, or 3.3, or 3.4, or 3.5, or 3.6, or 3.7, or 3.8, or 3.9, or 4, or 4.1, or 4.2, or 4.3, or 4.4, or 4.5, or 4.6, or 4.7, or 4.8, or 4.9, or 5, or 5.1, or 5.2, or 5.3, or 5.4, or 5.5, or 5.6, or 5.7, or 5.8, or 5.9, or 6% w/w hexyl acetate.

In some aspects, the extract further comprises from 3 to 15% w/w acetol. In some aspects, the extract further comprises from 3 to 14% w/w acetol. In some aspects, the extract further comprises from 3 to 13% w/w acetol. In some aspects, the extract further comprises from 3 to 12% w/w acetol. In some aspects, the extract further comprises from 3 to 11% w/w acetol. In some aspects, the extract further comprises from 3 to 10% w/w acetol. In some aspects, the extract further comprises from 3 to 9% w/w acetol. In some aspects, the extract further comprises from 3 to 8% w/w acetol. In some aspects, the extract further comprises from 3 to 7% w/w acetol. In some aspects, the extract further comprises from 3 to 6% w/w acetol. In some aspects, the extract further comprises from 3 to 5% w/w acetol. In some aspects, the extract further comprises from 3 to 4% w/w acetol.

In some aspects, the extract further comprises from 4 to 15% w/w acetol. In some aspects, the extract further comprises from 5 to 15% w/w acetol. In some aspects, the extract further comprises from 6 to 15% w/w acetol. In some aspects, the extract further comprises from 7 to 15% w/w acetol. In some aspects, the extract further comprises from 8 to 15% w/w acetol. In some aspects, the extract further comprises from 9 to 15% w/w acetol. In some aspects, the extract further comprises from 10 to 15% w/w acetol. In some aspects, the extract further comprises from 11 to 15% w/w acetol. In some aspects, the extract further comprises from 12 to 15% w/w acetol. In some aspects, the extract further comprises from 13 to 15% w/w acetol. In some aspects, the extract further comprises from 14 to 15% w/w acetol.

In some aspects, the extract further comprises 3, or 4, or 5, or 6, or 7, or 8, or 9, or 10, or 11, or 12, or 13, or 14, or 15% w/w acetol.

In some aspects, the extract further comprises from 2 to 8% w/w acetic acid. In some aspects, the extract further comprises from 3 to 8% w/w acetic acid. In some aspects, the extract further comprises from 4 to 8% w/w acetic acid. In some aspects, the extract further comprises from 5 to 8% w/w acetic acid. In some aspects, the extract further comprises from 6 to 8% w/w acetic acid. In some aspects, the extract further comprises from 7 to 8% w/w acetic acid.

In some aspects, the extract further comprises from 2 to 7% w/w acetic acid. In some aspects, the extract further comprises from 2 to 6% w/w acetic acid. In some aspects, the extract further comprises from 2 to 5% w/w acetic acid. In some aspects, the extract further comprises from 2 to 4% w/w acetic acid. In some aspects, the extract further comprises from 2 to 3% w/w acetic acid.

In some aspects, the extract further comprises 2, or 3, or 4, or 5, or 6, or 7, or 8% w/w acetic acid.

In some aspects, the extract further comprises from 0.5 to 2% w/w furfural. In some aspects, the extract further comprises from 1 to 2% w/w furfural. In some aspects, the extract further comprises from 1.5 to 2% w/w furfural.

In some aspects, the extract further comprises from 0.5 to 1.5% w/w furfural. In some aspects, the extract further comprises from 0.5 to 1% w/w furfural.

In some aspects, the extract further comprises 0.5, or 1, or 1.5, or 2% w/w furfural.

In some aspects, the extract further comprises from 0.5 to 2% w/w furfural alcohol. In some aspects, the extract further comprises from 1 to 2% w/w furfural alcohol. In some aspects, the extract further comprises from 1.5 to 2% w/w furfural alcohol.

In some aspects, the extract further comprises from 0.5 to 1.5% w/w furfural alcohol. In some aspects, the extract further comprises from 0.5 to 1% w/w furfural alcohol.

In some aspects, the extract further comprises 0.5, or 1, or 1.5, or 2% w/w furfural alcohol.

In some aspects, the extract further comprises from 0.2 to 2% w/w 2-methylbutyric acid. In some aspects, the extract further comprises from 0.4 to 2% w/w 2-methylbutyric acid. In some aspects, the extract further comprises from 0.6 to 2% w/w 2-methylbutyric acid. In some aspects, the extract further comprises from 0.8 to 2% w/w 2-methylbutyric acid. In some aspects, the extract further comprises from 1 to 2% w/w 2-methylbutyric acid. In some aspects, the extract further comprises from 1.2 to 2% w/w 2-methylbutyric acid. In some aspects, the extract further comprises from 1.4 to 2% w/w 2-methylbutyric acid. In some aspects, the extract further comprises from 1.6 to 2% w/w 2-methylbutyric acid. In some aspects, the extract further comprises from 1.8 to 2% w/w 2-methylbutyric acid.

In some aspects, the extract further comprises from 0.2 to 1.8% w/w 2-methylbutyric acid. In some aspects, the extract further comprises from 0.2 to 1.6% w/w 2-methylbutyric acid. In some aspects, the extract further comprises from 0.2 to 1.4% w/w 2-methylbutyric acid. In some aspects, the extract further comprises from 0.2 to 1.2% w/w 2-methylbutyric acid. In some aspects, the extract further comprises from 0.2 to 1% w/w 2-methylbutyric acid. In some aspects, the extract further comprises from 0.2 to 0.8% w/w 2-methylbutyric acid. In some aspects, the extract further comprises from 0.2 to 0.6% w/w 2-methylbutyric acid. In some aspects, the extract further comprises from 0.2 to 0.4% w/w 2-methylbutyric acid.

In some aspects, the extract further comprises 0.2, or 0.4, or 0.6, or 0.8, or 1, or 1.2, or 1.4, or 1.6, or 1.8, or 2% w/w 2-methylbutyric acid.

In some aspects, the extract further comprises from 6 to 46% w/w 1,3-dihydroxyacetone. In some aspects, the extract further comprises from 7 to 46% w/w 1,3-dihydroxyacetone. In some aspects, the extract further comprises from 8 to 46% w/w 1,3-dihydroxyacetone. In some aspects, the extract further comprises from 9 to 46% w/w 1,3-dihydroxyacetone. In some aspects, the extract further comprises from 10 to 46% w/w 1,3-dihydroxyacetone. In some aspects, the extract further comprises from 11 to 46% w/w 1,3-dihydroxyacetone. In some aspects, the extract further comprises from 12 to 46% w/w 1,3-dihydroxyacetone. In some aspects, the extract further comprises from 13 to 46% w/w 1,3-dihydroxyacetone. In some aspects, the extract further comprises from 14 to 46% w/w 1,3-dihydroxyacetone. In some aspects, the extract further comprises from 15 to 46% w/w 1,3-dihydroxyacetone. In some aspects, the extract further comprises from 16 to 46% w/w 1,3-dihydroxyacetone. In some aspects, the extract further comprises from 17 to 46% w/w 1,3-dihydroxyacetone. In some aspects, the extract further comprises from 18 to 46% w/w 1,3-dihydroxyacetone. In some aspects, the extract further comprises from 19 to 46% w/w 1,3-dihydroxyacetone. In some aspects, the extract further comprises from 20 to 46% w/w 1,3-dihydroxyacetone. In some aspects, the extract further comprises from 21 to 46% w/w 1,3-dihydroxyacetone. In some aspects, the extract further comprises from 22 to 46% w/w 1,3-dihydroxyacetone. In some aspects, the extract further comprises from 23 to 46% w/w 1,3-dihydroxyacetone. In some aspects, the extract further comprises from 24 to 46% w/w 1,3-dihydroxyacetone. In some aspects, the extract further comprises from 25 to 46% w/w 1,3-dihydroxyacetone. In some aspects, the extract further comprises from 26 to 46% w/w 1,3-dihydroxyacetone. In some aspects, the extract further comprises from 27 to 46% w/w 1,3-dihydroxyacetone. In some aspects, the extract further comprises from 28 to 46% w/w 1,3-dihydroxyacetone. In some aspects, the extract further comprises from 29 to 46% w/w 1,3-dihydroxyacetone. In some aspects, the extract further comprises from 30 to 46% w/w 1,3-dihydroxyacetone. In some aspects, the extract further comprises from 31 to 46% w/w 1,3-dihydroxyacetone. In some aspects, the extract further comprises from 32 to 46% w/w 1,3-dihydroxyacetone. In some aspects, the extract further comprises from 33 to 46% w/w 1,3-dihydroxyacetone. In some aspects, the extract further comprises from 34 to 46% w/w 1,3-dihydroxyacetone. In some aspects, the extract further comprises from 35 to 46% w/w 1,3-dihydroxyacetone. In some aspects, the extract further comprises from 36 to 46% w/w 1,3-dihydroxyacetone. In some aspects, the extract further comprises from 37 to 46% w/w 1,3-dihydroxyacetone. In some aspects, the extract further comprises from 38 to 46% w/w 1,3-dihydroxyacetone. In some aspects, the extract further comprises from 39 to 46% w/w 1,3-dihydroxyacetone. In some aspects, the extract further comprises from 40 to 46% w/w 1,3-dihydroxyacetone. In some aspects, the extract further comprises from 41 to 46% w/w 1,3-dihydroxyacetone. In some aspects, the extract further comprises from 42 to 46% w/w 1,3-dihydroxyacetone. In some aspects, the extract further comprises from 43 to 46% w/w 1,3-dihydroxyacetone. In some aspects, the extract further comprises from 44 to 46% w/w 1,3-dihydroxyacetone. In some aspects, the extract further comprises from 45 to 46% w/w 1,3-dihydroxyacetone.

In some aspects, the extract further comprises from 6 to 45% w/w 1,3-dihydroxyacetone. In some aspects, the extract further comprises from 6 to 44% w/w 1,3-dihydroxyacetone. In some aspects, the extract further comprises from 6 to 45% w/w 1,3-dihydroxyacetone. In some aspects, the extract further comprises from 6 to 42% w/w 1,3-dihydroxyacetone. In some aspects, the extract further comprises from 6 to 41% w/w 1,3-dihydroxyacetone. In some aspects, the extract further comprises from 6 to 40% w/w 1,3-dihydroxyacetone. In some aspects, the extract further comprises from 6 to 39% w/w 1,3-dihydroxyacetone. In some aspects, the extract further comprises from 6 to 38% w/w 1,3-dihydroxyacetone. In some aspects, the extract further comprises from 6 to 37% w/w 1,3-dihydroxyacetone. In some aspects, the extract further comprises from 6 to 36% w/w 1,3-dihydroxyacetone. In some aspects, the extract further comprises from 6 to 35% w/w 1,3-dihydroxyacetone. In some aspects, the extract further comprises from 6 to 34% w/w 1,3-dihydroxyacetone. In some aspects, the extract further comprises from 6 to 33% w/w 1,3-dihydroxyacetone. In some aspects, the extract further comprises from 6 to 32% w/w 1,3-dihydroxyacetone. In some aspects, the extract further comprises from 6 to 31% w/w 1,3-dihydroxyacetone. In some aspects, the extract further comprises from 6 to 30% w/w 1,3-dihydroxyacetone. In some aspects, the extract further comprises from 6 to 29% w/w 1,3-dihydroxyacetone. In some aspects, the extract further comprises from 6 to 28% w/w 1,3-dihydroxyacetone. In some aspects, the extract further comprises from 6 to 27% w/w 1,3-dihydroxyacetone. In some aspects, the extract further comprises from 6 to 26% w/w 1,3-dihydroxyacetone. In some aspects, the extract further comprises from 6 to 25% w/w 1,3-dihydroxyacetone. In some aspects, the extract further comprises from 6 to 24% w/w 1,3-dihydroxyacetone. In some aspects, the extract further comprises from 6 to 23% w/w 1,3-dihydroxyacetone. In some aspects, the extract further comprises from 6 to 22% w/w 1,3-dihydroxyacetone. In some aspects, the extract further comprises from 6 to 21% w/w 1,3-dihydroxyacetone. In some aspects, the extract further comprises from 6 to 20% w/w 1,3-dihydroxyacetone. In some aspects, the extract further comprises from 6 to 19% w/w 1,3-dihydroxyacetone. In some aspects, the extract further comprises from 6 to 18% w/w 1,3-dihydroxyacetone. In some aspects, the extract further comprises from 6 to 17% w/w 1,3-dihydroxyacetone. In some aspects, the extract further comprises from 6 to 16% w/w 1,3-dihydroxyacetone. In some aspects, the extract further comprises from 6 to 15% w/w 1,3-dihydroxyacetone. In some aspects, the extract further comprises from 6 to 14% w/w 1,3-dihydroxyacetone. In some aspects, the extract further comprises from 6 to 13% w/w 1,3-dihydroxyacetone. In some aspects, the extract further comprises from 6 to 12% w/w 1,3-dihydroxyacetone. In some aspects, the extract further comprises from 6 to 11% w/w 1,3-dihydroxyacetone. In some aspects, the extract further comprises from 6 to 10% w/w 1,3-dihydroxyacetone. In some aspects, the extract further comprises from 6 to 9% w/w 1,3-dihydroxyacetone. In some aspects, the extract further comprises from 6 to 8% w/w 1,3-dihydroxyacetone. In some aspects, the extract further comprises from 6 to 7% w/w 1,3-dihydroxyacetone.

In some aspects, the extract further comprises 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46% w/w 1,3-dihydroxyacetone.

In some aspects, the extract further comprises from 0.2 to 7% w/w 1,3-octanediol. In some aspects, the extract further comprises from 0.4 to 7% w/w 1,3-octanediol. In some aspects, the extract further comprises from 0.6 to 7% w/w 1,3-octanediol. In some aspects, the extract further comprises from 0.8 to 7% w/w 1,3-octanediol. In some aspects, the extract further comprises from 1 to 7% w/w 1,3-octanediol. In some aspects, the extract further comprises from 1.2 to 7% w/w 1,3-octanediol. In some aspects, the extract further comprises from 1.4 to 7% w/w 1,3-octanediol. In some aspects, the extract further comprises from 1.6 to 7% w/w 1,3-octanediol. In some aspects, the extract further comprises from 1.8 to 7% w/w 1,3-octanediol. In some aspects, the extract further comprises from 2 to 7% w/w 1,3-octanediol. In some aspects, the extract further comprises from 2.2 to 7% w/w 1,3-octanediol. In some aspects, the extract further comprises from 2.4 to 7% w/w 1,3-octanediol. In some aspects, the extract further comprises from 2.6 to 7% w/w 1,3-octanediol. In some aspects, the extract further comprises from 2.8 to 7% w/w 1,3-octanediol. In some aspects, the extract further comprises from 3 to 7% w/w 1,3-octanediol. In some aspects, the extract further comprises from 3.2 to 7% w/w 1,3-octanediol. In some aspects, the extract further comprises from 3.4 to 7% w/w 1,3-octanediol. In some aspects, the extract further comprises from 3.6 to 7% w/w 1,3-octanediol. In some aspects, the extract further comprises from 3.8 to 7% w/w 1,3-octanediol. In some aspects, the extract further comprises from 4 to 7% w/w 1,3-octanediol. In some aspects, the extract further comprises from 4.2 to 7% w/w 1,3-octanediol. In some aspects, the extract further comprises from 4.4 to 7% w/w 1,3-octanediol. In some aspects, the extract further comprises from 4.6 to 7% w/w 1,3-octanediol. In some aspects, the extract further comprises from 4.8 to 7% w/w 1,3-octanediol. In some aspects, the extract further comprises from 5 to 7% w/w 1,3-octanediol. In some aspects, the extract further comprises from 5.2 to 7% w/w 1,3-octanediol. In some aspects, the extract further comprises from 5.4 to 7% w/w 1,3-octanediol. In some aspects, the extract further comprises from 5.6 to 7% w/w 1,3-octanediol. In some aspects, the extract further comprises from 5.8 to 7% w/w 1,3-octanediol. In some aspects, the extract further comprises from 6 to 7% w/w 1,3-octanediol. In some aspects, the extract further comprises from 6.2 to 7% w/w 1,3-octanediol. In some aspects, the extract further comprises from 6.4 to 7% w/w 1,3-octanediol. In some aspects, the extract further comprises from 6.6 to 7% w/w 1,3-octanediol. In some aspects, the extract further comprises from 6.8 to 7% w/w 1,3-octanediol.

In some aspects, the extract further comprises from 0.2 to 6.8% w/w 1,3-octanediol. In some aspects, the extract further comprises from 0.2 to 6.4% w/w 1,3-octanediol. In some aspects, the extract further comprises from 0.2 to 6.2% w/w 1,3-octanediol. In some aspects, the extract further comprises from 0.2 to 6% w/w 1,3-octanediol. In some aspects, the extract further comprises from 0.2 to 5.8% w/w 1,3-octanediol. In some aspects, the extract further comprises from 0.2 to 5.4% w/w 1,3-octanediol. In some aspects, the extract further comprises from 0.2 to 5.2% w/w 1,3-octanediol. In some aspects, the extract further comprises from 0.2 to 5% w/w 1,3-octanediol. In some aspects, the extract further comprises from 0.2 to 4.8% w/w 1,3-octanediol. In some aspects, the extract further comprises from 0.2 to 4.4% w/w 1,3-octanediol. In some aspects, the extract further comprises from 0.2 to 4.2% w/w 1,3-octanediol. In some aspects, the extract further comprises from 0.2 to 4% w/w 1,3-octanediol. In some aspects, the extract further comprises from 0.2 to 3.8% w/w 1,3-octanediol. In some aspects, the extract further comprises from 0.2 to 3.4% w/w 1,3-octanediol. In some aspects, the extract further comprises from 0.2 to 3.2% w/w 1,3-octanediol. In some aspects, the extract further comprises from 0.2 to 3% w/w 1,3-octanediol. In some aspects, the extract further comprises from 0.2 to 2.8% w/w 1,3-octanediol. In some aspects, the extract further comprises from 0.2 to 2.4% w/w 1,3-octanediol. In some aspects, the extract further comprises from 0.2 to 2.2% w/w 1,3-octanediol. In some aspects, the extract further comprises from 0.2 to 2% w/w 1,3-octanediol. In some aspects, the extract further comprises from 0.2 to 1.8% w/w 1,3-octanediol. In some aspects, the extract further comprises from 0.2 to 1.4% w/w 1,3-octanediol. In some aspects, the extract further comprises from 0.2 to 1.2% w/w 1,3-octanediol. In some aspects, the extract further comprises from 0.2 to 1% w/w 1,3-octanediol. In some aspects, the extract further comprises from 0.2 to 0.8% w/w 1,3-octanediol. In some aspects, the extract further comprises from 0.2 to 0.4% w/w 1,3-octanediol.

In some aspects, the extract further comprises 0.2, or 0.4, or 0.6, or 0.8, or 1, or 1.2, or 1.4, or 1.6, or 1.8, or 2, or 2.2, or 2.4, or 2.6, or 2.8, or 3, or 3.2, or 3.4, or 3.6, or 3.8, or 4, or 4.2, or 4.4, or 4.6, or 4.8, or 5, or 5.2, or 5.4, or 5.6, or 5.8, or 6, or 6.2, or 6.4, or 6.6, or 6.8, or 7% w/w 1,3-octanediol.

In some aspects, the extract further comprises from 0.5 to 4% w/w 5-hydroxy-5,6-dihydrolmaltol. In some aspects, the extract further comprises from 1 to 4% w/w 5-hydroxy-5,6-dihydrolmaltol. In some aspects, the extract further comprises from 1.5 to 4% w/w 5-hydroxy-5,6-dihydrolmaltol. In some aspects, the extract further comprises from 2 to 4% w/w 5-hydroxy-5,6-dihydrolmaltol. In some aspects, the extract further comprises from 2.5 to 4% w/w 5-hydroxy-5,6-dihydrolmaltol. In some aspects, the extract further comprises from 3 to 4% w/w 5-hydroxy-5,6-dihydrolmaltol. In some aspects, the extract further comprises from 3.5 to 4% w/w 5-hydroxy-5,6-dihydrolmaltol.

In some aspects, the extract further comprises from 0.5 to 3.5% w/w 5-hydroxy-5,6-dihydrolmaltol. In some aspects, the extract further comprises from 0.5 to 3% w/w 5-hydroxy-5,6-dihydrolmaltol. In some aspects, the extract further comprises from 0.5 to 2.5% w/w 5-hydroxy-5,6-dihydrolmaltol. In some aspects, the extract further comprises from 0.5 to 2% w/w 5-hydroxy-5,6-dihydrolmaltol. In some aspects, the extract further comprises from 0.5 to 1.5% w/w 5-hydroxy-5,6-dihydrolmaltol. In some aspects, the extract further comprises from 0.5 to 1% w/w 5-hydroxy-5,6-dihydrolmaltol.

In some aspects, the extract further comprises 0.5, or 1, or 1.5, or 2, or 2.5, or 3, or 3.5, or 4% w/w 5-hydroxy-5,6-dihydrolmaltol.

In some aspects, the extract further comprises from 1 to 4% w/w 5-hydroxymethyl furfural. In some aspects, the extract further comprises from 2 to 4% w/w 5-hydroxymethyl furfural. In some aspects, the extract further comprises from 3 to 4% w/w 5-hydroxymethyl furfural.

In some aspects, the extract further comprises from 1 to 3% w/w 5-hydroxymethyl furfural. In some aspects, the extract further comprises from 1 to 2% w/w 5-hydroxymethyl furfural.

In some aspects, the extract further comprises 1, or 2, or 3, or 4% w/w 5-hydroxymethyl furfural.

In some aspects, the extract comprises the compounds, at the amounts disclosed in any one of Microwave Extract 1, or Microwave Extract 2 disclosed in Table 3.

Extract Derived from Green Tea Leaves: One aspect presented herein provides an extract from green tea leaves, wherein the extract comprises at least 12% w/w caffeine.

In some aspects, the extract comprises from 12 to 30% w/w caffeine.

In some aspects, the extract further comprises less than 8% furfuryl alcohol.

In some aspects, the extract further comprises 3,5-octadien-2-one, β-ionone, and 4,5-epoxy-β-ionone.

In some aspects, the extract comprises the compounds, at the amounts disclosed in any one of Microwave Extract 1, or Microwave Extract 2 disclosed in Table 4.

Extract Derived from Roasted Coffee Beans: One aspect presented herein provides an extract from roasted coffee beans, wherein the extract comprises at least 2% w/w coffee furanone.

In some aspects, the extract comprises from 2 to 2.5% w/w coffee furanone. In some aspects, the extract comprises from 2.1 to 2.5% w/w coffee furanone. In some aspects, the extract comprises from 2.2 to 2.5% w/w coffee furanone. In some aspects, the extract comprises from 2.3 to 2.5% w/w coffee furanone. In some aspects, the extract comprises from 2.4 to 2.5% w/w coffee furanone.

In some aspects, the extract comprises from 2 to 2.4% w/w coffee furanone. In some aspects, the extract comprises from 2 to 2.3% w/w coffee furanone. In some aspects, the extract comprises from 2 to 2.2% w/w coffee furanone. In some aspects, the extract comprises from 2 to 2.1% w/w coffee furanone.

In some aspects, the extract comprises 2, or 2.1, or 2.2, or 2.3, or 2.4, or 2.5% w/w coffee furanone.

In some aspects, the extract further comprises from 3 to 4% w/w 2-methyl pyrazine. In some aspects, the extract further comprises from 3.1 to 4% w/w 2-methyl pyrazine. In some aspects, the extract further comprises from 3.2 to 4% w/w 2-methyl pyrazine. In some aspects, the extract further comprises from 3.3 to 4% w/w 2-methyl pyrazine. In some aspects, the extract further comprises from 3.4 to 4% w/w 2-methyl pyrazine. In some aspects, the extract further comprises from 3.5 to 4% w/w 2-methyl pyrazine. In some aspects, the extract further comprises from 3.6 to 4% w/w 2-methyl pyrazine. In some aspects, the extract further comprises from 3.7 to 4% w/w 2-methyl pyrazine. In some aspects, the extract further comprises from 3.8 to 4% w/w 2-methyl pyrazine. In some aspects, the extract further comprises from 3.9 to 4% w/w 2-methyl pyrazine.

In some aspects, the extract further comprises from 3 to 3.9% w/w 2-methyl pyrazine. In some aspects, the extract further comprises from 3 to 3.8% w/w 2-methyl pyrazine. In some aspects, the extract further comprises from 3 to 3.7% w/w 2-methyl pyrazine. In some aspects, the extract further comprises from 3 to 3.6% w/w 2-methyl pyrazine. In some aspects, the extract further comprises from 3 to 3.5% w/w 2-methyl pyrazine. In some aspects, the extract further comprises from 3 to 3.4% w/w 2-methyl pyrazine. In some aspects, the extract further comprises from 3 to 3.3% w/w 2-methyl pyrazine. In some aspects, the extract further comprises from 3 to 3.2% w/w 2-methyl pyrazine. In some aspects, the extract further comprises from 3 to 3.1% w/w 2-methyl pyrazine.

In some aspects, the extract further comprises 3, or 3.1, or 3.2, or 3.3, or 3.4, or 3.5, or 3.6, or 3.7, or 3.8, or 3.9, or 4% w/w 2-methyl pyrazine.

In some aspects, the extract further comprises from 0.5 to 2% w/w acetylmethyl carbinol. In some aspects, the extract further comprises from 1 to 2% w/w acetylmethyl carbinol. In some aspects, the extract further comprises from 1.5 to 2% w/w acetylmethyl carbinol.

In some aspects, the extract further comprises from 0.5 to 1.5% w/w acetylmethyl carbinol. In some aspects, the extract further comprises from 0.5 to 1% w/w acetylmethyl carbinol.

In some aspects, the extract further comprises 0.5, or 1, or 1.5, or 2% w/w acetylmethyl carbinol.

In some aspects, the extract further comprises from 0.5 to 8% w/w acetol. In some aspects, the extract further comprises from 1 to 8% w/w acetol. In some aspects, the extract further comprises from 1.5 to 8% w/w acetol. In some aspects, the extract further comprises from 2 to 8% w/w acetol. In some aspects, the extract further comprises from 2.5 to 8% w/w acetol. In some aspects, the extract further comprises from 3 to 8% w/w acetol. In some aspects, the extract further comprises from 3.5 to 8% w/w acetol. In some aspects, the extract further comprises from 4 to 8% w/w acetol. In some aspects, the extract further comprises from 4.5 to 8% w/w acetol. In some aspects, the extract further comprises from 5 to 8% w/w acetol. In some aspects, the extract further comprises from 5.5 to 8% w/w acetol. In some aspects, the extract further comprises from 6 to 8% w/w acetol. In some aspects, the extract further comprises from 6.5 to 8% w/w acetol. In some aspects, the extract further comprises from 7 to 8% w/w acetol. In some aspects, the extract further comprises from 7.5 to 8% w/w acetol.

In some aspects, the extract further comprises from 0.5 to 7.5% w/w acetol. In some aspects, the extract further comprises from 0.5 to 7% w/w acetol. In some aspects, the extract further comprises from 0.5 to 6.5% w/w acetol. In some aspects, the extract further comprises from 0.5 to 6% w/w acetol. In some aspects, the extract further comprises from 0.5 to 5.5% w/w acetol. In some aspects, the extract further comprises from 0.5 to 5% w/w acetol. In some aspects, the extract further comprises from 0.5 to 4.5% w/w acetol. In some aspects, the extract further comprises from 0.5 to 4% w/w acetol. In some aspects, the extract further comprises from 0.5 to 3.5% w/w acetol. In some aspects, the extract further comprises from 0.5 to 5% w/w acetol. In some aspects, the extract further comprises from 0.5 to 2.5% w/w acetol. In some aspects, the extract further comprises from 0.5 to 2% w/w acetol. In some aspects, the extract further comprises from 0.5 to 1.5% w/w acetol. In some aspects, the extract further comprises from 0.5 to 1% w/w acetol.

In some aspects, the extract further comprises 0.5, or 1, or 1.5, or 2, or 2.5, or 3, or 3.5, or 4, or 4.5, or 5, or 5.5, or 6, or 6.5, or 7, or 7.5, or 8% w/w acetol.

In some aspects, the extract further comprises from 35 to 45% w/w furfuryl alcohol. In some aspects, the extract further comprises from 36 to 45% w/w furfuryl alcohol. In some aspects, the extract further comprises from 37 to 45% w/w furfuryl alcohol. In some aspects, the extract further comprises from 38 to 45% w/w furfuryl alcohol. In some aspects, the extract further comprises from 39 to 45% w/w furfuryl alcohol. In some aspects, the extract further comprises from 40 to 45% w/w furfuryl alcohol. In some aspects, the extract further comprises from 41 to 45% w/w furfuryl alcohol. In some aspects, the extract further comprises from 42 to 45% w/w furfuryl alcohol. In some aspects, the extract further comprises from 43 to 45% w/w furfuryl alcohol. In some aspects, the extract further comprises from 44 to 45% w/w furfuryl alcohol.

In some aspects, the extract further comprises from 35 to 44% w/w furfuryl alcohol. In some aspects, the extract further comprises from 35 to 43% w/w furfuryl alcohol. In some aspects, the extract further comprises from 35 to 42% w/w furfuryl alcohol. In some aspects, the extract further comprises from 35 to 41% w/w furfuryl alcohol. In some aspects, the extract further comprises from 35 to 40% w/w furfuryl alcohol. In some aspects, the extract further comprises from 35 to 39% w/w furfuryl alcohol. In some aspects, the extract further comprises from 35 to 38% w/w furfuryl alcohol. In some aspects, the extract further comprises from 35 to 37% w/w furfuryl alcohol. In some aspects, the extract further comprises from 35 to 36% w/w furfuryl alcohol.

In some aspects, the extract further comprises 35, or 36, or 37, or 38, or 39, or 40, or 41, or 42, or 43, or 44, or 45% w/w furfuryl alcohol.

In some aspects, the extract further comprises 0.2% w/w 2-cyclopentenone.

In some aspects, the extract comprises the compounds, at the amounts disclosed in any one of Microwave Extract 1, or Microwave Extract 2 disclosed in Table 6.

In some aspects, the extract comprises from 0.8 ppm to 20 ppm caffeine. In some aspects, the extract comprises from 0.8 ppm to 19 ppm caffeine. In some aspects, the extract comprises from 0.8 ppm to 18 ppm caffeine. In some aspects, the extract comprises from 0.8 ppm to 17 ppm caffeine. In some aspects, the extract comprises from 0.8 ppm to 16 ppm caffeine. In some aspects, the extract comprises from 0.8 ppm to 15 ppm caffeine. In some aspects, the extract comprises from 0.8 ppm to 14 ppm caffeine. In some aspects, the extract comprises from 0.8 ppm to 13 ppm caffeine. In some aspects, the extract comprises from 0.8 ppm to 12 ppm caffeine. In some aspects, the extract comprises from 0.8 ppm to 11 ppm caffeine. In some aspects, the extract comprises from 0.8 ppm to 10 ppm caffeine. In some aspects, the extract comprises from 0.8 ppm to 9 ppm caffeine. In some aspects, the extract comprises from 0.8 ppm to 8 ppm caffeine. In some aspects, the extract comprises from 0.8 ppm to 7 ppm caffeine. In some aspects, the extract comprises from 0.8 ppm to 6 ppm caffeine. In some aspects, the extract comprises from 0.8 ppm to 5 ppm caffeine. In some aspects, the extract comprises from 0.8 ppm to 4 ppm caffeine. In some aspects, the extract comprises from 0.8 ppm to 3 ppm caffeine. In some aspects, the extract comprises from 0.8 ppm to 2 ppm caffeine. In some aspects, the extract comprises from 0.8 ppm to 1 ppm caffeine. In some aspects, the extract comprises from 0.8 ppm to 0.9 ppm caffeine.

In some aspects, the extract comprises from 0.9 ppm to 20 ppm caffeine. In some aspects, the extract comprises from 1 ppm to 20 ppm caffeine. In some aspects, the extract comprises from 2 ppm to 20 ppm caffeine. In some aspects, the extract comprises from 3 ppm to 20 ppm caffeine. In some aspects, the extract comprises from 4 ppm to 20 ppm caffeine. In some aspects, the extract comprises from 5 ppm to 20 ppm caffeine. In some aspects, the extract comprises from 6 ppm to 20 ppm caffeine. In some aspects, the extract comprises from 7 ppm to 20 ppm caffeine. In some aspects, the extract comprises from 8 ppm to 20 ppm caffeine. In some aspects, the extract comprises from 9 ppm to 20 ppm caffeine. In some aspects, the extract comprises from 10 ppm to 20 ppm caffeine. In some aspects, the extract comprises from 11 ppm to 20 ppm caffeine. In some aspects, the extract comprises from 12 ppm to 20 ppm caffeine. In some aspects, the extract comprises from 13 ppm to 20 ppm caffeine. In some aspects, the extract comprises from 14 ppm to 20 ppm caffeine. In some aspects, the extract comprises from 15 ppm to 20 ppm caffeine. In some aspects, the extract comprises from 16 ppm to 20 ppm caffeine. In some aspects, the extract comprises from 17 ppm to 20 ppm caffeine. In some aspects, the extract comprises from 18 ppm to 20 ppm caffeine. In some aspects, the extract comprises from 19 ppm to 20 ppm caffeine.

In some aspects, the extract comprises 0.8, or 0.9, or 1, or 2, or 3, or 4, or 5, or 6, or 7, or 8, or 9, or 10, or 11, or 12, or 13, or 14, or 15, or 16, or 17, or 18, or 19, or 20 ppm caffeine.

In some aspects, the extract comprises from 5 ppm to 40 ppm theobromine. In some aspects, the extract comprises from 5 ppm to 35 ppm theobromine. In some aspects, the extract comprises from 5 ppm to 30 ppm theobromine. In some aspects, the extract comprises from 5 ppm to 25 ppm theobromine. In some aspects, the extract comprises from 5 ppm to 20 ppm theobromine. In some aspects, the extract comprises from 5 ppm to 15 ppm theobromine. In some aspects, the extract comprises from 5 ppm to 10 ppm theobromine.

In some aspects, the extract comprises from 10 ppm to 40 ppm theobromine. In some aspects, the extract comprises from 15 ppm to 40 ppm theobromine. In some aspects, the extract comprises from 20 ppm to 40 ppm theobromine. In some aspects, the extract comprises from 25 ppm to 40 ppm theobromine. In some aspects, the extract comprises from 30 ppm to 40 ppm theobromine. In some aspects, the extract comprises from 35 ppm to 40 ppm theobromine.

In some aspects, the extract comprises 5, or 10, or 15, or 20, or 25, or 30, or 35, or 40 ppm theobromine.

In some aspects, the extract comprises from 10 ppm to 200 ppm theophylline. In some aspects, the extract comprises from 10 ppm to 190 ppm theophylline. In some aspects, the extract comprises from 10 ppm to 180 ppm theophylline. In some aspects, the extract comprises from 10 ppm to 170 ppm theophylline. In some aspects, the extract comprises from 10 ppm to 160 ppm theophylline. In some aspects, the extract comprises from 10 ppm to 150 ppm theophylline. In some aspects, the extract comprises from 10 ppm to 140 ppm theophylline. In some aspects, the extract comprises from 10 ppm to 130 ppm theophylline. In some aspects, the extract comprises from 10 ppm to 120 ppm theophylline. In some aspects, the extract comprises from 10 ppm to 110 ppm theophylline. In some aspects, the extract comprises from 10 ppm to 100 ppm theophylline. In some aspects, the extract comprises from 10 ppm to 90 ppm theophylline. In some aspects, the extract comprises from 10 ppm to 80 ppm theophylline. In some aspects, the extract comprises from 10 ppm to 70 ppm theophylline. In some aspects, the extract comprises from 10 ppm to 60 ppm theophylline. In some aspects, the extract comprises from 10 ppm to 50 ppm theophylline. In some aspects, the extract comprises from 10 ppm to 40 ppm theophylline. In some aspects, the extract comprises from 10 ppm to 30 ppm theophylline. In some aspects, the extract comprises from 10 ppm to 20 ppm theophylline.

In some aspects, the extract comprises from 20 ppm to 200 ppm theophylline. In some aspects, the extract comprises from 30 ppm to 200 ppm theophylline. In some aspects, the extract comprises from 40 ppm to 200 ppm theophylline. In some aspects, the extract comprises from 50 ppm to 200 ppm theophylline. In some aspects, the extract comprises from 60 ppm to 200 ppm theophylline. In some aspects, the extract comprises from 70 ppm to 200 ppm theophylline. In some aspects, the extract comprises from 80 ppm to 200 ppm theophylline. In some aspects, the extract comprises from 90 ppm to 200 ppm theophylline. In some aspects, the extract comprises from 100 ppm to 200 ppm theophylline. In some aspects, the extract comprises from 110 ppm to 200 ppm theophylline. In some aspects, the extract comprises from 120 ppm to 200 ppm theophylline. In some aspects, the extract comprises from 130 ppm to 200 ppm theophylline. In some aspects, the extract comprises from 140 ppm to 200 ppm theophylline. In some aspects, the extract comprises from 150 ppm to 200 ppm theophylline. In some aspects, the extract comprises from 160 ppm to 200 ppm theophylline. In some aspects, the extract comprises from 170 ppm to 200 ppm theophylline. In some aspects, the extract comprises from 180 ppm to 200 ppm theophylline. In some aspects, the extract comprises from 190 ppm to 200 ppm theophylline.

In some aspects, the extract comprises 10, or 20, or 30, or 40, or 50, or 60, or 70, or 80, or 90, or 100, or 110, or 120, or 130, or 140, or 150, or 160, ort 70, or 180, or 190, or 200 ppm theophylline.

Without intending to be limited to any particular theory, the differences in the coffee extract obtained according to a method according to the aspects presented herein differs from a commercially obtained extract in volatile compounds that contribute to the sweet notes and olfactive profile of fresh coffee.

In some aspects, the methods according to certain aspects presented herein increase the amount of non-volatile compounds in the extract, compared to extracts generated by other methods in the art.

Examples of non-volatile compounds increased by a method according to certain aspects presented herein include, but are not limited to anthocyanins, anthocyanidins, and the like.

Referring to Table 7, one aspect presented herein provides an extract of a plant biomass, wherein the concentration of at least one anthocyanin is 1 ppm. In some aspects, the concentration of at least one anthocyanin in the extract is 0.9 ppm. In some aspects, the concentration of at least one anthocyanin in the extract is 0.8 ppm. In some aspects, the concentration of at least one anthocyanin in the extract is 0.7 ppm. In some aspects, the concentration of at least one anthocyanin in the extract is 0.6 ppm. In some aspects, the concentration of at least one anthocyanin in the extract is 0.5 ppm. In some aspects, the concentration of at least one anthocyanin in the extract is 0.4 ppm. In some aspects, the concentration of at least one anthocyanin in the extract is 0.3 ppm. In some aspects, the concentration of at least one anthocyanin in the extract is 0.2 ppm. In some aspects, the concentration of at least one anthocyanin in the extract is 0.1 ppm. In some aspects, the concentration of at least one anthocyanin in the extract is 0.09 ppm. In some aspects, the concentration of at least one anthocyanin in the extract is 0.08 ppm. In some aspects, the concentration of at least one anthocyanin in the extract is 0.07 ppm. In some aspects, the concentration of at least one anthocyanin in the extract is 0.06 ppm. In some aspects, the concentration of at least one anthocyanin in the extract is 0.05 ppm.

As used herein, the term “anthocyanin” refers to water-soluble vacuolar flavonoid pigments that, depending on their pH, may appear red, purple, or blue. Food plants rich in anthocyanins include the blueberry, raspberry, black rice, and black soybean, among many others that are red, blue, purple, or black. Anthocyanins occur in all tissues of higher plants, including leaves, stems, roots, flowers, and fruits. Anthocyanins are derived from anthocyanidins via the addition of sugar residues.

Referring to Table 7, one aspect presented herein provides an extract of a plant biomass, wherein the concentration of at least one anthocyanidin is 1 ppm. In some aspects, the concentration of at least one anthocyanidin in the extract is 0.9 ppm. In some aspects, the concentration of at least one anthocyanidin in the extract is 0.8 ppm. In some aspects, the concentration of at least one anthocyanidin in the extract is 0.7 ppm. In some aspects, the concentration of at least one anthocyanidin in the extract is 0.6 ppm. In some aspects, the concentration of at least one anthocyanidin in the extract is 0.5 ppm. In some aspects, the concentration of at least one anthocyanidin in the extract is 0.4 ppm. In some aspects, the concentration of at least one anthocyanidin in the extract is 0.3 ppm. In some aspects, the concentration of at least one anthocyanidin in the extract is 0.2 ppm. In some aspects, the concentration of at least one anthocyanidin in the extract is 0.1 ppm. In some aspects, the concentration of at least one anthocyanidin in the extract is 0.09 ppm. In some aspects, the concentration of at least one anthocyanidin in the extract is 0.08 ppm. In some aspects, the concentration of at least one anthocyanidin in the extract is 0.07 ppm. In some aspects, the concentration of at least one anthocyanidin in the extract is 0.06 ppm. In some aspects, the concentration of at least one anthocyanidin in the extract is 0.05 ppm.

In some aspects, the at least one anthocyanidin is selected from the group consisting of: cyanidin, delphinidin, malvinidin, peonidin, petunidin, and combinations thereof.

Additional anthocyanidins include: aurantinidin, europinidin, pelargonidin, and rosinidin.

In some aspects, the at least one anthocyanin is selected from the group consisting of: cyanidin-3-arabinoside, cyanidin-3-galactoside, cyanidin-3-glucoside, delphinidin-3-arabinoside, delphinidin-3-galactoside, delphinidin-3-glucoside, malvinidin-3-arabinoside, malvinidin-3-galactoside, malvinidin-3-glucoside, peonidin-3-arabinoside, peonidin-3-galactoside, peonidin-3-glucoside, petunidin-3-arabinoside, petunidin-3-galactoside, petunidin-3-glucoside, and combinations thereof.

In some aspects, the concentration of cyanidin-3-glucoside in the extract is 0.5 ppm.

In some aspects, the concentration of peonidin in the extract is 0.07 ppm.

In some aspects, the concentration of peonidin-3-galactoside in the extract is 0.2 ppm.

In some aspects, the concentration of peonidin-3-glucoside in the extract is 0.8 ppm.

In some aspects, the concentration of petunidin in the extract is 0.6 ppm.

In some aspects, the extract comprises 0.5 ppm cyanidin-3-glucoside, 0.07 ppm peonidin, 0.2 ppm peonidin-3-galactoside, 0.8 ppm peonidin-3-glucoside, and 0.6 ppm petunidin.

In some aspects, the methods according to certain aspects presented herein increase the concentration of at least one compound selected from the group consisting of: caffeine, theobromine, theophylline, cyanidin, delphinidin, malvinidin, peonidin, petunidin, cyanidin-3-arabinoside, cyanidin-3-galactoside, cyanidin-3-glucoside, delphinidin-3-arabinoside, delphinidin-3-galactoside, delphinidin-3-glucoside, malvinidin-3-arabinoside, malvinidin-3-galactoside, malvinidin-3-glucoside, peonidin-3-arabinoside, peonidin-3-galactoside, peonidin-3-glucoside, petunidin-3-arabinoside, petunidin-3-galactoside, petunidin-3-glucoside, and combinations thereof.

In some aspects, the methods according to certain aspects presented herein increase the sugar content of the extract, compared to extracts generated by other methods in the art. Without intending to be limited to any particular theory, the increase in the amount of the sugar content in the extract according to the methods presented herein does not require the addition of organic solvents, such as, for example, ethanol.

Examples of sugars increased include reducing sugars, monosaccharides, disaccharides, and the like.

In some aspects, the extract comprises reducing sugars in an amount from 2 to 2.5 g/100 g of the extract. In some aspects, the extract comprises reducing sugars in an amount from 2.1 to 2.5 g/100 g of the extract. In some aspects, the extract comprises reducing sugars in an amount from 2.2 to 2.5 g/100 g of the extract. In some aspects, the extract comprises reducing sugars in an amount from 2.3 to 2.5 g/100 g of the extract. In some aspects, the extract comprises reducing sugars in an amount from 2.4 to 2.5 g/100 g of the extract.

In some aspects, the extract comprises reducing sugars in an amount from 2 to 2.5 g/100 g of the extract. In some aspects, the extract comprises reducing sugars in an amount from 2 to 2.4 g/100 g of the extract. In some aspects, the extract comprises reducing sugars in an amount from 2 to 2.3 g/100 g of the extract. In some aspects, the extract comprises reducing sugars in an amount from 2 to 2.2 g/100 g of the extract. In some aspects, the extract comprises reducing sugars in an amount from 2 to 2.1 g/100 g of the extract.

In some aspects, the extract comprises 2, or 2.1, or 2.2, or 2.3, or 2.4, or 2.5 g reducing sugars/100 g of the extract.

In some aspects, the extract comprises mono and disaccharides in an amount from 2 to 3.5 g/100 g of the extract. In some aspects, the extract comprises mono and disaccharides in an amount from 2.1 to 3.5 g/100 g of the extract. In some aspects, the extract comprises mono and disaccharides in an amount from 2.2 to 3.5 g/100 g of the extract. In some aspects, the extract comprises mono and disaccharides in an amount from 2.3 to 3.5 g/100 g of the extract. In some aspects, the extract comprises mono and disaccharides in an amount from 2.4 to 3.5 g/100 g of the extract. In some aspects, the extract comprises mono and disaccharides in an amount from 2.5 to 3.5 g/100 g of the extract. In some aspects, the extract comprises mono and disaccharides in an amount from 2.6 to 3.5 g/100 g of the extract. In some aspects, the extract comprises mono and disaccharides in an amount from 2.7 to 3.5 g/100 g of the extract. In some aspects, the extract comprises mono and disaccharides in an amount from 2.8 to 3.5 g/100 g of the extract. In some aspects, the extract comprises mono and disaccharides in an amount from 2.9 to 3.5 g/100 g of the extract. In some aspects, the extract comprises mono and disaccharides in an amount from 3 to 3.5 g/100 g of the extract. In some aspects, the extract comprises mono and disaccharides in an amount from 3.1 to 3.5 g/100 g of the extract. In some aspects, the extract comprises mono and disaccharides in an amount from 3.2 to 3.5 g/100 g of the extract. In some aspects, the extract comprises mono and disaccharides in an amount from 3.3 to 3.5 g/100 g of the extract. In some aspects, the extract comprises mono and disaccharides in an amount from 3.4 to 3.5 g/100 g of the extract.

In some aspects, the extract comprises mono and disaccharides in an amount from 2.1 to 3.5 g/100 g of the extract. In some aspects, the extract comprises mono and disaccharides in an amount from 2.1 to 3.4 g/100 g of the extract. In some aspects, the extract comprises mono and disaccharides in an amount from 2.1 to 3.3 g/100 g of the extract. In some aspects, the extract comprises mono and disaccharides in an amount from 2.1 to 3.2 g/100 g of the extract. In some aspects, the extract comprises mono and disaccharides in an amount from 2.1 to 3.1 g/100 g of the extract. In some aspects, the extract comprises mono and disaccharides in an amount from 2.1 to 3 g/100 g of the extract. In some aspects, the extract comprises mono and disaccharides in an amount from 2.1 to 2.9 g/100 g of the extract. In some aspects, the extract comprises mono and disaccharides in an amount from 2.1 to 2.8 g/100 g of the extract. In some aspects, the extract comprises mono and disaccharides in an amount from 2.1 to 2.7 g/100 g of the extract. In some aspects, the extract comprises mono and disaccharides in an amount from 2.1 to 2.6 g/100 g of the extract. In some aspects, the extract comprises mono and disaccharides in an amount from 2.1 to 2.5 g/100 g of the extract. In some aspects, the extract comprises mono and disaccharides in an amount from 2.1 to 2.4 g/100 g of the extract. In some aspects, the extract comprises mono and disaccharides in an amount from 2.1 to 2.3 g/100 g of the extract. In some aspects, the extract comprises mono and disaccharides in an amount from 2.1 to 2.2 g/100 g of the extract.

In some aspects, the extract comprises 2, or 2.1, or 2.2, or 2.3, or 2.4, or 2.5, or 2.6, or 2.7, or 2.8, or 2.9, or 3, or 3.1, or 3.2, or 3.3, or 3.4, or 3.5 g mono and disaccharides/100 g of the extract.

In some aspects, the extract comprises glucose in an amount greater than 1 g/100 g of the extract.

In some aspects, the extract comprises glucose in an amount from 0.9 to 1.1 g/100 g of the extract. In some aspects, the extract comprises glucose in an amount from 1 to 1.1 g/100 g of the extract.

In some aspects, the extract comprises glucose in an amount from 0.9 to 1 g/100 g of the extract.

In some aspects, the extract comprises 0.9, or 1, or 1.1 g glucose/100 g of the extract.

In some aspects, the extract comprises fructose in an amount from 1.1 to 1.5 g/100 g of the extract. In some aspects, the extract comprises fructose in an amount from 1.2 to 1.5 g/100 g of the extract. In some aspects, the extract comprises fructose in an amount from 1.3 to 1.5 g/100 g of the extract. In some aspects, the extract comprises fructose in an amount from 1.4 to 1.5 g/100 g of the extract.

In some aspects, the extract comprises fructose in an amount from 1.1 to 1.4 g/100 g of the extract. In some aspects, the extract comprises fructose in an amount from 1.1 to 1.3 g/100 g of the extract. In some aspects, the extract comprises fructose in an amount from 1.2 to 1.5 g/100 g of the extract.

In some aspects, the extract comprises 1.1, or 1.2, or 1.3, or 1.4, or 1.5 g fructose/100 g of the extract.

In some aspects, the extract comprises sucrose in an amount from 0.2 to 1 g/100 g of the extract. In some aspects, the extract comprises sucrose in an amount from 0.3 to 1 g/100 g of the extract. In some aspects, the extract comprises sucrose in an amount from 0.4 to 1 g/100 g of the extract. In some aspects, the extract comprises sucrose in an amount from 0.5 to 1 g/100 g of the extract. In some aspects, the extract comprises sucrose in an amount from 0.6 to 1 g/100 g of the extract. In some aspects, the extract comprises sucrose in an amount from 0.7 to 1 g/100 g of the extract. In some aspects, the extract comprises sucrose in an amount from 0.8 to 1 g/100 g of the extract. In some aspects, the extract comprises sucrose in an amount from 0.9 to 1 g/100 g of the extract.

In some aspects, the extract comprises sucrose in an amount from 0.2 to 0.9 g/100 g of the extract. In some aspects, the extract comprises sucrose in an amount from 0.2 to 0.8 g/100 g of the extract. In some aspects, the extract comprises sucrose in an amount from 0.2 to 0.7 g/100 g of the extract. In some aspects, the extract comprises sucrose in an amount from 0.2 to 0.6 g/100 g of the extract. In some aspects, the extract comprises sucrose in an amount from 0.2 to 0.5 g/100 g of the extract. In some aspects, the extract comprises sucrose in an amount from 0.2 to 0.4 g/100 g of the extract. In some aspects, the extract comprises sucrose in an amount from 0.2 to 0.3 g/100 g of the extract.

In some aspects, the extract comprises 0.2, or 0.3, or 0.4, or 0.5, or 0.6, or 0.7, or 0.8, or 0.9, or 1 g sucrose/100 g of the extract.

Extract from strawberry. In some aspects, the plant biomass is strawberry.

In the accompanying examples the inventors prepared a strawberry extract using the method of the invention.

Here it can be seen that the strawberry extract of the invention has a surprising aroma profile in comparison with the classic strawberry extract. In particular, the strawberry extract of the invention has a more authentic strawberry aroma profile in respect of fruity, creamy, jammy and floral aspects.

One aspect is wherein the wherein the strawberry extract comprises at least 0.01 ppm 2,3 butanedione.

2,3 butanedione is a ketone and contributes to the creamy aroma profile of the strawberry extract of the invention. As can be seen from the data in the accompanying examples, 2,3 butanedione is not detectable in the classic strawberry extract.

In some aspects the extract comprises from 0.007 ppm to 0.027 ppm 2,3 butanedione. In some aspects the extract comprises from 0.008 ppm to 0.027 ppm 2,3 butanedione. In some aspects the extract comprises from 0.009 ppm to 0.027 ppm 2,3 butanedione. In some aspects the extract comprises from 0.010 ppm to 0.027 ppm 2,3 butanedione. In some aspects the extract comprises from 0.011 ppm to 0.027 ppm 2,3 butanedione. In some aspects the extract comprises from 0.012 ppm to 0.027 ppm 2,3 butanedione. In some aspects the extract comprises from 0.013 ppm to 0.027 ppm 2,3 butanedione. In some aspects the extract comprises from 0.014 ppm to 0.027 ppm 2,3 butanedione. In some aspects the extract comprises from 0.015 ppm to 0.027 ppm 2,3 butanedione. In some aspects the extract comprises from 0.016 ppm to 0.027 ppm 2,3 butanedione. In some aspects the extract comprises from 0.017 ppm to 0.027 ppm 2,3 butanedione. In some aspects the extract comprises from 0.018 ppm to 0.027 ppm 2,3 butanedione. In some aspects the extract comprises from 0.019 ppm to 0.027 ppm 2,3 butanedione. In some aspects the extract comprises from 0.020 ppm to 0.027 ppm 2,3 butanedione. In some aspects the extract comprises from 0.021 ppm to 0.027 ppm 2,3 butanedione. In some aspects the extract comprises from 0.022 ppm to 0.027 ppm 2,3 butanedione. In some aspects the extract comprises from 0.023 ppm to 0.027 ppm 2,3 butanedione. In some aspects the extract comprises from 0.024 ppm to 0.027 ppm 2,3 butanedione. In some aspects the extract comprises from 0.025 ppm to 0.027 ppm 2,3 butanedione. In some aspects the extract comprises from 0.026 ppm to 0.027 ppm 2,3 butanedione.

In some aspects the extract comprises from 0.007 ppm to 0.026 ppm 2,3 butanedione. In some aspects the extract comprises from 0.007 ppm to 0.025 ppm 2,3 butanedione. In some aspects the extract comprises from 0.007 ppm to 0.024 ppm 2,3 butanedione. In some aspects the extract comprises from 0.007 ppm to 0.023 ppm 2,3 butanedione. In some aspects the extract comprises from 0.007 ppm to 0.022 ppm 2,3 butanedione. In some aspects the extract comprises from 0.007 ppm to 0.021 ppm 2,3 butanedione. In some aspects the extract comprises from 0.007 ppm to 0.020 ppm 2,3 butanedione. In some aspects the extract comprises from 0.007 ppm to 0.019 ppm 2,3 butanedione. In some aspects the extract comprises from 0.007 ppm to 0.018 ppm 2,3 butanedione. In some aspects the extract comprises from 0.007 ppm to 0.017 ppm 2,3 butanedione. In some aspects the extract comprises from 0.007 ppm to 0.016 ppm 2,3 butanedione. In some aspects the extract comprises from 0.007 ppm to 0.015 ppm 2,3 butanedione. In some aspects the extract comprises from 0.007 ppm to 0.014 ppm 2,3 butanedione. In some aspects the extract comprises from 0.007 ppm to 0.013 ppm 2,3 butanedione. In some aspects the extract comprises from 0.007 ppm to 0.012 ppm 2,3 butanedione. In some aspects the extract comprises from 0.007 ppm to 0.011 ppm 2,3 butanedione. In some aspects the extract comprises from 0.007 ppm to 0.010 ppm 2,3 butanedione. In some aspects the extract comprises from 0.007 ppm to 0.09 ppm 2,3 butanedione. In some aspects the extract comprises from 0.007 ppm to 0.08 ppm 2,3 butanedione.

In some aspects the extract comprises from 0.05 ppm to 0.1 ppm 3,7,11-trimethyl-1,6,10-Dodecatrien-3-ol.

3,7,11-trimethyl-1,6,10-Dodecatrien-3-ol is a sesquiterpene alcohol and contributes to the floral aroma property of the strawberry extract of the invention. As can be seen from the data in the accompanying examples 3,7,11-trimethyl-1,6,10-Dodecatrien-3-ol is not detectable in the classic strawberry extract.

In some aspects the extract comprises from 0.06 ppm to 0.1 ppm 3,7,11-trimethyl-1,6,10-Dodecatrien-3-ol. In some aspects the extract comprises from 0.065 ppm to 0.1 ppm 3,7,11-trimethyl-1,6,10-Dodecatrien-3-ol. In some aspects the extract comprises from 0.066 ppm to 0.1 ppm 3,7,11-trimethyl-1,6,10-Dodecatrien-3-ol. In some aspects the extract comprises from 0.067 ppm to 0.1 ppm 3,7,11-trimethyl-1,6,10-Dodecatrien-3-ol. In some aspects the extract comprises from 0.068 ppm to 0.1 ppm 3,7,11-trimethyl-1,6,10-Dodecatrien-3-ol. In some aspects the extract comprises from 0.69 ppm to 0.1 ppm 3,7,11-trimethyl-1,6,10-Dodecatrien-3-ol. In some aspects the extract comprises from 0.70 ppm to 0.1 ppm 3,7,11-trimethyl-1,6,10-Dodecatrien-3-ol. In some aspects the extract comprises from 0.71 ppm to 0.1 ppm 3,7,11-trimethyl-1,6,10-Dodecatrien-3-ol. In some aspects the extract comprises from 0.072 ppm to 0.1 ppm 3,7,11-trimethyl-1,6,10-Dodecatrien-3-ol. In some aspects the extract comprises from 0.073 ppm to 0.1 ppm 3,7,11-trimethyl-1,6,10-Dodecatrien-3-ol. In some aspects the extract comprises from 0.074 ppm to 0.1 ppm 3,7,11-trimethyl-1,6,10-Dodecatrien-3-ol. In some aspects the extract comprises from 0.075 ppm to 0.1 ppm 3,7,11-trimethyl-1,6,10-Dodecatrien-3-ol. In some aspects the extract comprises from 0.076 ppm to 0.1 ppm 3,7,11-trimethyl-1,6,10-Dodecatrien-3-ol. In some aspects the extract comprises from 0.077 ppm to 0.1 ppm 3,7,11-trimethyl-1,6,10-Dodecatrien-3-ol. In some aspects the extract comprises from 0.078 ppm to 0.1 ppm 3,7,11-trimethyl-1,6,10-Dodecatrien-3-ol. In some aspects the extract comprises from 0.079 ppm to 0.1 ppm 3,7,11-trimethyl-1,6,10-Dodecatrien-3-ol. In some aspects the extract comprises from 0.08 ppm to 0.1 ppm 3,7,11-trimethyl-1,6,10-Dodecatrien-3-ol. In some aspects the extract comprises from 0.081 ppm to 0.1 ppm 3,7,11-trimethyl-1,6,10-Dodecatrien-3-ol. In some aspects the extract comprises from 0.082 ppm to 0.1 ppm 3,7,11-trimethyl-1,6,10-Dodecatrien-3-ol. In some aspects the extract comprises from 0.083 ppm to 0.1 ppm 3,7,11-trimethyl-1,6,10-Dodecatrien-3-ol. In some aspects the extract comprises from 0.084 ppm to 0.1 ppm 3,7,11-trimethyl-1,6,10-Dodecatrien-3-ol. In some aspects the extract comprises from 0.085 ppm to 0.1 ppm 3,7,11-trimethyl-1,6,10-Dodecatrien-3-ol. In some aspects the extract comprises from 0.09 ppm to 0.1 ppm 3,7,11-trimethyl-1,6,10-Dodecatrien-3-ol. In some aspects the extract comprises from 0.095 ppm to 0.1 ppm 3,7,11-trimethyl-1,6,10-Dodecatrien-3-ol.

In some aspects the extract comprises from 0.05 ppm to 0.09 ppm 3,7,11-trimethyl-1,6,10-Dodecatrien-3-ol. In some aspects the extract comprises from 0.05 ppm to 0.085 ppm 3,7,11-trimethyl-1,6,10-Dodecatrien-3-ol. In some aspects the extract comprises from 0.05 ppm to 0.084 ppm 3,7,11-trimethyl-1,6,10-Dodecatrien-3-ol. In some aspects the extract comprises from 0.05 ppm to 0.083 ppm 3,7,11-trimethyl-1,6,10-Dodecatrien-3-ol. In some aspects the extract comprises from 0.05 ppm to 0.082 ppm 3,7,11-trimethyl-1,6,10-Dodecatrien-3-ol. In some aspects the extract comprises from 0.05 ppm to 0.081 ppm 3,7,11-trimethyl-1,6,10-Dodecatrien-3-ol. In some aspects the extract comprises from 0.05 ppm to 0.080 ppm 3,7,11-trimethyl-1,6,10-Dodecatrien-3-ol. In some aspects the extract comprises from 0.05 ppm to 0.079 ppm 3,7,11-trimethyl-1,6,10-Dodecatrien-3-ol. In some aspects the extract comprises from 0.05 ppm to 0.078 ppm 3,7,11-trimethyl-1,6,10-Dodecatrien-3-ol. In some aspects the extract comprises from 0.05 ppm to 0.077 ppm 3,7,11-trimethyl-1,6,10-Dodecatrien-3-ol. In some aspects the extract comprises from 0.05 ppm to 0.076 ppm 3,7,11-trimethyl-1,6,10-Dodecatrien-3-ol. In some aspects the extract comprises from 0.05 ppm to 0.075 ppm 3,7,11-trimethyl-1,6,10-Dodecatrien-3-ol. In some aspects the extract comprises from 0.05 ppm to 0.074 ppm 3,7,11-trimethyl-1,6,10-Dodecatrien-3-ol. In some aspects the extract comprises from 0.05 ppm to 0.073 ppm 3,7,11-trimethyl-1,6,10-Dodecatrien-3-ol. In some aspects the extract comprises from 0.05 ppm to 0.072 ppm 3,7,11-trimethyl-1,6,10-Dodecatrien-3-ol. In some aspects the extract comprises from 0.05 ppm to 0.071 ppm 3,7,11-trimethyl-1,6,10-Dodecatrien-3-ol. In some aspects the extract comprises from 0.05 ppm to 0.07 ppm 3,7,11-trimethyl-1,6,10-Dodecatrien-3-ol. In some aspects the extract comprises from 0.05 ppm to 0.069 ppm 3,7,11-trimethyl-1,6,10-Dodecatrien-3-ol. In some aspects the extract comprises from 0.05 ppm to 0.068 ppm 3,7,11-trimethyl-1,6,10-Dodecatrien-3-ol. In some aspects the extract comprises from 0.05 ppm to 0.067 ppm 3,7,11-trimethyl-1,6,10-Dodecatrien-3-ol. In some aspects the extract comprises from 0.05 ppm to 0.066 ppm 3,7,11-trimethyl-1,6,10-Dodecatrien-3-ol. In some aspects the extract comprises from 0.05 ppm to 0.65 ppm 3,7,11-trimethyl-1,6,10-Dodecatrien-3-ol. In some aspects the extract comprises from 0.05 ppm to 0.06 ppm 3,7,11-trimethyl-1,6,10-Dodecatrien-3-ol. In some aspects the extract comprises from 0.05 ppm to 0.055 ppm 3,7,11-trimethyl-1,6,10-Dodecatrien-3-ol.

In some aspects the extract comprises from 0.02 to 0.1 ppm dihydro-5-octyl-2(3H)-Furanone.

Dihydro-5-octyl-2(3H)-Furanone is a lactone and contributes to the fleshy and fruity aroma property of the strawberry extract of the invention. As can be seen from the data in the accompanying examples dihydro-5-octyl-2(3H)-Furanone is detectable in the classic strawberry extract at around 0.01 ppm.

In some aspects the extract comprises from 0.03 to 0.1 ppm dihydro-5-octyl-2(3H)-Furanone. In some aspects the extract comprises from 0.04 to 0.1 ppm dihydro-5-octyl-2(3H)-Furanone. In some aspects the extract comprises from 0.05 to 0.1 ppm dihydro-5-octyl-2(3H)-Furanone. In some aspects the extract comprises from 0.06 to 0.1 ppm dihydro-5-octyl-2(3H)-Furanone. In some aspects the extract comprises from 0.07 to 0.1 ppm dihydro-5-octyl-2(3H)-Furanone. In some aspects the extract comprises from 0.08 to 0.1 ppm dihydro-5-octyl-2(3H)-Furanone. In some aspects the extract comprises from 0.09 to 0.1 ppm dihydro-5-octyl-2(3H)-Furanone. In some aspects the extract comprises from 0.02 to 0.09 ppm dihydro-5-octyl-2(3H)-Furanone. In some aspects the extract comprises from 0.02 to 0.08 ppm dihydro-5-octyl-2(3H)-Furanone. In some aspects the extract comprises from 0.02 to 0.07 ppm dihydro-5-octyl-2(3H)-Furanone. In some aspects the extract comprises from 0.02 to 0.06 ppm dihydro-5-octyl-2(3H)-Furanone. In some aspects the extract comprises from 0.02 to 0.05 ppm dihydro-5-octyl-2(3H)-Furanone. In some aspects the extract comprises from 0.02 to 0.04 ppm dihydro-5-octyl-2(3H)-Furanone. In some aspects the extract comprises from 0.02 to 0.03 ppm dihydro-5-octyl-2(3H)-Furanone.

In some aspects the extract comprises 0.05 to 0.15 ppm 4-hydroxy-2,5-dimethyl-3(2H)-Furanone.

4-hydroxy-2,5-dimethyl-3(2H)-Furanone is a alicyclic unsaturated ketone and contributes to the fruity, jammy aroma property of the strawberry extract of the invention. As can be seen from the data in the accompanying examples 4-hydroxy-2,5-dimethyl-3(2H)-Furanone is not detectable in the classic strawberry extract.

In some aspects the extract comprises 0.06 to 0.15 ppm 4-hydroxy-2,5-dimethyl-3(2H)-Furanone. In some aspects the extract comprises 0.07 to 0.15 ppm 4-hydroxy-2,5-dimethyl-3(2H)-Furanone. In some aspects the extract comprises 0.08 to 0.15 ppm 4-hydroxy-2,5-dimethyl-3(2H)-Furanone. In some aspects the extract comprises 0.09 to 0.15 ppm 4-hydroxy-2,5-dimethyl-3(2H)-Furanone. In some aspects the extract comprises 0.1 to 0.15 ppm 4-hydroxy-2,5-dimethyl-3(2H)-Furanone. In some aspects the extract comprises 0.11 to 0.15 ppm 4-hydroxy-2,5-dimethyl-3(2H)-Furanone. In some aspects the extract comprises 0.12 to 0.15 ppm 4-hydroxy-2,5-dimethyl-3(2H)-Furanone. In some aspects the extract comprises 0.13 to 0.15 ppm 4-hydroxy-2,5-dimethyl-3(2H)-Furanone. In some aspects the extract comprises 0.14 to 0.15 ppm 4-hydroxy-2,5-dimethyl-3(2H)-Furanone. In some aspects the extract comprises 0.05 to 0.14 ppm 4-hydroxy-2,5-dimethyl-3(2H)-Furanone. In some aspects the extract comprises 0.05 to 0.13 ppm 4-hydroxy-2,5-dimethyl-3(2H)-Furanone. In some aspects the extract comprises 0.05 to 0.12 ppm 4-hydroxy-2,5-dimethyl-3(2H)-Furanone. In some aspects the extract comprises 0.05 to 0.11 ppm 4-hydroxy-2,5-dimethyl-3(2H)-Furanone. In some aspects the extract comprises 0.05 to 0.1 ppm 4-hydroxy-2,5-dimethyl-3(2H)-Furanone. In some aspects the extract comprises 0.05 to 0.09 ppm 4-hydroxy-2,5-dimethyl-3(2H)-Furanone. In some aspects the extract comprises 0.05 to 0.08 ppm 4-hydroxy-2,5-dimethyl-3(2H)-Furanone. In some aspects the extract comprises 0.05 to 0.07 ppm 4-hydroxy-2,5-dimethyl-3(2H)-Furanone. In some aspects the extract comprises 0.05 to 0.06 ppm 4-hydroxy-2,5-dimethyl-3(2H)-Furanone.

In some aspects the extract comprises less than 0.3 ppm 2-methylpropan-1-ol.

2-methylpropan-1-ol is a saturated aliphatic alcohol and has an alcoholic, fermented aroma. This is not a desirable aroma property. While 2-methylpropan-1-ol is present in over 0.3 ppm in the classic strawberry extract, the strawberry extract of the invention has much less of this compound.

In some aspects the extract comprises less than 0.25 ppm 2-methylpropan-1-ol. In some aspects the extract comprises less than 0.2 ppm 2-methylpropan-1-ol. In some aspects the extract comprises less than 0.15 ppm 2-methylpropan-1-ol. In some aspects the extract comprises less than 0.1 ppm 2-methylpropan-1-ol. In some aspects the extract comprises less than 0.05 ppm 2-methylpropan-1-ol. In some aspects the extract comprises less than 0.03 ppm 2-methylpropan-1-ol. In some aspects the extract comprises less than 0.02 ppm 2-methylpropan-1-ol. In some aspects the extract comprises less than 0.01 ppm 2-methylpropan-1-ol. In some aspects the extract comprises less than detectable 2-methylpropan-1-ol.

In some aspects the extract comprises less than 0.5 ppm 3-methyl butanol.

3-methyl butanol is a saturated aliphatic alcohol and has an alcoholic, fermented aroma. This is not a desirable aroma property. While 3-methyl butanol is present at around 0.5 ppm in the classic strawberry extract, the strawberry extract of the invention has much less of this compound.

In some aspects the extract comprises less than 0.3 ppm 3-methyl butanol. In some aspects the extract comprises less than 0.1 ppm 3-methyl butanol. In some aspects the extract comprises less than 0.05 ppm 3-methyl butanol. In some aspects the extract comprises less than 0.03 ppm 3-methyl butanol. In some aspects the extract comprises less than 0.02 ppm 3-methyl butanol. In some aspects the extract comprises less than 0.01 ppm 3-methyl butanol. In some aspects the extract comprises less than detectable 3-methyl butanol.

In some aspects the extract comprises less than 0.4 ppm 2-hexenal.

2-hexenal is a saturated aliphatic unsaturated aldehyde and has an green apple aroma. This is not a desirable aroma property. While 2-hexenal is present at around 0.45 ppm in the classic strawberry extract, the strawberry extract of the invention has much less of this compound.

In some aspects the extract comprises less than 0.3 ppm 2-hexenal. In some aspects the extract comprises less than 0.2 ppm 2-hexenal. In some aspects the extract comprises less than 0.1 ppm 2-hexenal. In some aspects the extract comprises less than 0.05 ppm 2-hexenal. In some aspects the extract comprises less than 0.03 ppm 2-hexenal. In some aspects the extract comprises less than 0.02 ppm 2-hexenal. In some aspects the extract comprises less than 0.01 ppm 2-hexenal. In some aspects the extract comprises less than detectable 2-hexenal.

A further aspect of the invention provides a strawberry extract comprising one or more of the following features: (i) 0.007 ppm to 0.027 ppm 2,3 butanedione, (ii) 0.05 ppm to 0.1 ppm 3,7,11-trimethyl-1,6,10-Dodecatrien-3-ol; (iii) 0.02 to 0.1 ppm dihydro-5-octyl-2(3H)-Furanone; (iv) 0.05 to 0.15 ppm 4-hydroxy-2,5-dimethyl-3(2H)-Furanone.

A preferred embodiment of this aspect of the invention is wherein the strawberry extract comprises: (i) 0.007 ppm to 0.027 ppm 2,3 butanedione, (ii) 0.05 ppm to 0.1 ppm 3,7,11-trimethyl-1,6,10-Dodecatrien-3-ol; (iii) 0.02 to 0.1 ppm dihydro-5-octyl-2(3H)-Furanone; and (iv) 0.05 to 0.15 ppm 4-hydroxy-2,5-dimethyl-3(2H)-Furanone.

A further aspect of the invention provides a strawberry extract comprising one or more of the following features: (i) less than 0.3 ppm 2-methylpropan-1-ol; (ii) less than 0.5 ppm 3-methyl butanol; (iii) less than 0.4 ppm 2-hexenal.

A preferred embodiment of this aspect of the invention is wherein the strawberry extract comprises (i) less than 0.3 ppm 2-methylpropan-1-ol; (ii) less than 0.5 ppm 3-methyl butanol; and (iii) less than 0.4 ppm 2-hexenal.

A further aspect of the invention provides an strawberry extract comprising: (i) 0.007 ppm to 0.027 ppm 2,3 butanedione, (ii) 0.05 ppm to 0.1 ppm 3,7,11-trimethyl-1,6,10-Dodecatrien-3-ol; (iii) 0.02 to 0.1 ppm dihydro-5-octyl-2(3H)-Furanone; (iv) 0.05 to 0.15 ppm 4-hydroxy-2,5-dimethyl-3(2H)-Furanone, (v) less than 0.3 ppm 2-methylpropan-1-ol; (vi) less than 0.5 ppm 3-methyl butanol; and (vii) less than 0.4 ppm 2-hexenal.

A further aspect of the invention provides a strawberry extract comprising the compounds, at the amounts disclosed in Table 8 in the column labeled Strawberry Eco-extract/juiceppm, RTD.

### Compositions Comprising an Extract According to Some Aspects Presented Herein

It is understood by a person skilled in the art that an extract according to some aspects presented herein may be added into a composition described herein in neat form, or in a solvent. Alternatively, an extract according to some aspects presented herein may first be modified, for example by entrapped with an entrapment material such as for example polymers, capsules, microcapsules, nanocapsules, liposomes, precursors, film formers, absorbents such as for example by using carbon or zeolites, cyclic oligosaccharides and mixtures thereof.

Accordingly, some aspects presented herein provide a composition comprising:
a. an extract according to some aspects presented herein;
b. at least one ingredient selected from the group consisting of a perfumery carrier and a perfumery base; and
c. optionally at least one perfumery adjuvant.

As used herein, the term “perfumery carrier” refers to a material which is practically neutral from a perfumery point of view, i.e. which does not significantly alter the organoleptic properties of perfuming ingredients. The perfumery carrier may be a liquid or a solid.

Non-limiting examples of liquid perfumery carriers include an emulsifying system, i.e. a solvent and a surfactant system, or a solvent commonly used in perfumery. A detailed description of the nature and type of solvents commonly used in perfumery cannot be exhaustive. However, non-limiting examples solvents include dipropyleneglycol, diethyl phthalate, isopropyl myristate, benzyl benzoate, 2-(2-ethoxyethoxy)-1-ethanol or ethyl citrate. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company).

Non-limiting examples of solid perfumery carriers include absorbing gums or polymers, or yet encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs- and Geliermittel in Lebensmitteln, Band 2 der Schriftenreihe Lebensmittelchemie, Lebensmittelqualitat, Behr's Verlag GmbH & Co., Hamburg, 1996. The encapsulation is a well-known process to a person skilled in the art, and may be performed, for instance, using techniques such as spray-drying, agglomeration or yet extrusion; or consists of a coating encapsulation, including coacervation and complex coacervation technique.

As used herein, the term “perfumery base” refers a composition comprising at least one perfuming co-ingredient. A perfuming co-ingredient does not include a compound of formula (I). As used herein, the term “perfuming co-ingredient” refers to compound, which is used in a perfuming preparation or a composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of his general knowledge and according to intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

As used herein, the term “perfumery adjuvant” refers to an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Accordingly, in some aspects, the present disclosure provides a perfumed consumer product comprising an effective amount of an extract according to some aspects presented herein. In some aspects, the perfumed consumer product is selected from the group consisting of: air care products, home care products and laundry care products.

In some aspects, the perfumed consumer product comprising an effective amount of an extract according to some aspects presented herein comprises a formulation selected from the group consisting of: aerosol and/or water-based air freshener spray, wick/reed air freshener, liquid electrical (plug-in) air freshener, a solid support air freshener, gel-based air freshener, membrane-containing air freshener, bleaching, cleaning, washing detergent powder, liquid all-purpose cleaner, specialty cleaner and liquid detergent.

It should be appreciated by those skilled in the art that the conception and the specific embodiments disclosed might be readily utilized as a basis for modifying or formulating other formulations for carrying the same purposes of the present invention. It should also be realized by those skilled in the art that such equivalent formulations do not depart from the spirit and scope of the disclosure as set forth in the appended claims.

The proportions in which an extract according to some aspects presented herein can be incorporated into the various aforementioned products or compositions vary within a wide range of values. These values are dependent on the nature of the consumer product and on the desired organoleptic effect as well as the nature of the co-ingredients in a given composition when the extract according to some aspects presented herein are mixed with other ingredients, solvents or additives commonly used in the art.

In general, for example, in the case of perfuming compositions, typical concentrations are in the order of 0.001% to 5% by weight, or even more, of an extract according to some aspects presented herein, based on the weight of the composition into which they are incorporated. Concentrations lower than these, such as in the order of 0.01% to 100% by weight, can be used when the compositions described herein are incorporated into consumer products, the percentage being relative to the weight of the consumer product.

In particular, the concentration of a composition according to the aspects described herein, used in the various aforementioned consumer products varies within a various wide range of values depending on the nature of the consumer product. For instance, a composition according some aspects described herein can be used in a perfume product at a concentration of 0.01% to 50% by weight, alternatively at a concentration of 0.2% to 40% by weight, alternatively at a concentration of 0.5% to 25% by weight. For instance, a composition according to some aspects described herein can be used in a fabric care product at a concentration of 0.01% to 20% by weight, alternatively at a concentration of 0.05% to 10% by weight, alternatively at a concentration of 0.1% to 5% by weight. For instance, a composition according to some aspects described herein can be used in a hair care product at a concentration of 0.01% to 10% by weight, alternatively at a concentration of 0.05% to 5% by weight, alternatively at a concentration of 0.1% to 3% by weight. For instance, a composition according to some aspects described herein can be used in a skin care product at a concentration of 0.01% to 10% by weight, alternatively at a concentration of 0.05% to 5% by weight, most preferably at a concentration of 0.1% to 2.5% by weight. For instance, a composition according to some aspects described herein can be used in a body deodorant or antiperspirant product at a concentration of 0.01% to 10% by weight, alternatively at a concentration of 0.05% to 7% by weight, alternatively at a concentration of 0.1% to 5% by weight. For instance, a composition according to some aspects described herein can be used in a skin cleansing product at a concentration of 0.01% to 5% by weight, alternatively at a concentration of 0.05% to 3% by weight, alternatively at a concentration of 0.1% to 2.5% by weight. For instance, a composition according to some aspects described herein can be used in an air freshening product at a concentration of 0.01% to 100% by weight. For instance, a composition according to some aspects described herein can be used in a surface care product at a concentration of 0.001% to 10% by weight, alternatively at a concentration of 0.01% to 5% by weight, alternatively at a concentration of 0.1% to 2% by weight. Yet, for instance, a composition according to some aspects described herein can be used in a pet-litter product at a concentration of 0.001% to 1% by weight, alternatively at a concentration of 0.005% to 0.5% by weight, alternatively at a concentration of 0.01% to 0.3% by weight.

In some aspects, the composition as defined in any of the above aspect may be absorbed on a porous or non-porous substrate in loose powder or compacted form, the substrate being selected from cellulose (paper/cardboard), vermiculite, other industrial absorbents, perlite, calcium carbonate, pumice, wood, sawdust, ground corn cob, ground rice hull, rice hull ash, biochars, starches, modified starches and mixtures thereof.

One aspect presented herein provides a perfumed article containing the essential oil according to some aspects presented herein, wherein the perfumed article is selected from the group consisting of: an aromatic water, a perfume, a cologne, a bath gel, a shower gel, a shampoo, a hair care product, a cosmetic preparation, a deodorant, an air freshener, a detergent, a fabric softener, and a household cleaner.

Non-limiting examples of suitable consumer product include:
a perfume, such as a fine perfume, an Eau de Toilette, a cologne or an after-shave lotion;
a fabric care product, such as a liquid detergent, a powder detergent, detergent tablets, a detergent bar, a detergent paste, a liquid fabric softener, fabric softener sheets, a fabric scent booster, a laundry pre-treatment, a fabric refresher, an ironing water, a laundry bleach, a carpet powder or a carpet cleaner;
a hair care product, such as a shampoo, a hair conditioner, a hair cream, a hair oil, a hair styling product (such as a spray, mousse or gel), a hair coloration product or a hair permanent wave product;
a skin care product, such as a face cream, a face lotion, a shaving product (such as a foam, cream, gel or oil), a body and/or hand product (such as a lotion, cream, gel or oil), a skin firming product, a depilatory, a talcum powder, a foot care cream or lotion, baby wipes, cleansing wipes, moisturizer wipes, a sun-protection product (such as a spray, lotion, cream or oil), an after-sun lotion, or a self-tanning product;
a body deodorant or antiperspirant product, such as a body deodorant spray, a roll-on deodorant, a deodorant stick, a deodorant cream, an antiperspirant spray, an antiperspirant stick, a roll-on antiperspirant liquid, an antiperspirant stick, or an antiperspirant cream;
a skin-cleansing product, such as a soap bar, a shower gel, a liquid hand soap, a bath foam or an intimate wash product;
an air freshening product, such as an air freshener spray, a gel air freshener, a liquid-wick air freshener, a solid air freshener comprising a porous substrate (such as a paper or card blotter, a porous ceramic, or a porous plastic), a liquid or gel air freshener comprising a permeable membrane, an electrically operated air freshener, and a dual purpose air freshener/disinfectant spray; and/or
a surface care product, such as an all-purpose cleaner, a furniture polish, a wood floor cleaner, a window cleaner, a hand dishwashing product (such as a liquid, gel or paste), a machine dishwashing product (such as a powder, liquid, gel, tablet or sachet), a toilet bowl cleaning liquid, an in-cistern toilet cleaner, a toilet rim block, or a toilet rim liquid; a pet-litter

As outlined above the invention the extract of a plant biomass may be from a fruit, for example pears or strawberry.

Hence another object of the invention is the use of the extract as a flavouring composition. In other words, it concerns a method or a process to confer, enhance, improve or modify the flavoring or a flavored article or, which method comprises adding to said composition an effective amount of the extract, e.g. to impart its typical note.

By “use of the invention's flavouring composition” it has to be understood here also the use of any composition containing the invention's flavouring composition and which can be advantageously employed in the flavor industry.

Furthermore, the invention's flavouring composition can be advantageously used in all the fields of flavor to positively impart or modify the taste of a consumer product into which said composition is added. Consequently, the present invention relates to a flavored consumer product comprising the invention's composition as defined above.

For the sake of clarity, by “flavored consumer product” it is meant to designate an edible product or oral composition such as, for example, pharmaceutical compositions, edible gel mixes and compositions, dental compositions, foodstuffs beverages and beverage products. The flavored consumer product may be in a different form. A non-exhaustive list of suitable form of the consumer product may include fried, frozen, marinated, battered, chilled, dehydrated, powder blended, canned, reconstituted, retorted, baked, cooked, fermented, microfiltred, pasteurized, blended or preserved. Therefore, a flavored consumer product according to the invention comprises the invention's composition, as well as optional benefit agents, corresponding to taste and flavor profile of the desired edible product, e.g. a cream dessert.

The nature and type of the constituents of the foodstuffs or beverages do not warrant a more detailed description here, the skilled person being able to select them on the basis of his general knowledge and according to the nature of said product.

Typical examples of said flavored consumer product include:
Baked goods (e.g. breads, dry biscuits, cakes, rice cakes, rice crackers, cookies, crackers, donuts, muffins, pastries, pre-mixes, other baked goods),
Non-alcoholic beverages (e.g. aqueous beverages, enhanced/slightly sweetened water drinks, flavored carbonated and still mineral and table waters, carbonated soft drinks, non-carbonated beverages, carbonated waters, still waters, softs, bottled waters, sports/energy drinks, juice drinks, vegetable juices, vegetable juice preparations, broth drinks),
Alcoholic beverages (e.g. beer and malt beverages, spirituous beverages, wines, liquors),
Instant or ready-to-drink beverages (e.g. instant vegetable drinks, powdered soft drinks, instant coffees and teas, black teas, green teas, oolong teas, herbal infusions, cacaos (e.g. water-based), tea-based drinks, coffee-based drinks, cacao-based drinks, infusions, syrups, frozen fruits, frozen fruit juices, water-based ices, fruit ices, sorbets),
Cereal products (e.g. breakfast cereals, cereal bars, energy bars/nutritional bars, granolas, pre-cooked ready-made rice products, rice flour products, millet and sorghum products, raw or pre-cooked noodles and pasta products),
Dairy based products (e.g. fruit or flavored yoghurts, ice creams, fruit ices, frozen desserts, fresh cheeses, soft cheeses, hard cheeses, milk drinks, wheys, butters, partially or wholly hydrolysed milk protein-containing products, fermented milk products, condensed milks and analogues)
Dairy analogues (imitation dairy products) containing non-dairy ingredients (plant-based proteins, vegetable fats),
Confectionary products (e.g. filings, toppings, chewing gums, hard and soft candies),
Chocolate and compound coatings (e.g. chocolates, spreads),
Products based on fat and oil or emulsions thereof (e.g. mayonnaises, spreads, regular or low fat margarines, butter/margarine blends, flavored oils, shortenings, remoulades, dressings, salad dressings, spice preparations, peanut butters),
Eggs or egg products (dried eggs, egg whites, egg yolks, custards),
Desserts (e.g. gelatins, puddings, dessert creams),
Products made of soya protein or other soya bean fractions (e.g. soya milk and products made therefrom, soya lecithin-containing preparations, fermented products such as tofu or tempeh or products manufactured therefrom, soya sauces),
Vegetable preparations (e.g. ketchups, sauces, processed and reconstituted vegetables, dried vegetables, deep frozen vegetables, pre-cooked vegetables, vegetables pickled in vinegar, vegetable concentrates or pastes, cooked vegetables, potato preparations),
Fruit preparations (e.g. jams, marmalades, canned fruits)
Vegetarian meat analogues or meat replacers, vegetarian burgers
Spices or spice preparations (e.g. mustard preparations, horseradish preparations, pickles), spice mixtures and, in particular seasonings which are used, for example, in the field of snacks.
Snack articles (e.g. baked or fried potato crisps or potato dough products, bread dough products, extrudates based on maize, rice or ground nuts),
Ready dishes (e.g. instant noodles, rice, pastas, pizzas, tortillas, wraps) and soups and broths (e.g. stock, savory cubes, dried soups, instant soups, pre-cooked soups, retorted soups), sauces (instant sauces, dried sauces, ready-made sauces, gravies, sweet sauces, a relish sauces, a sour sauces),
oral care product, such as toothpastes, mouth washes, dental care products (e.g. denture adhesives), dental rinsing, mouth sprays, dental powders, dental gels or dental floss,
pet or animal food.

Preferably, the flavored consumer product may be baked goods, dairy based products, dairy analogues, products based on fat and oil or emulsions thereof, milk products, confectionary products, desserts, chocolate and compound coatings, cereal products, non-alcoholic beverages, alcoholic beverages or instant or ready-to-drink beverages.

Preferably, the flavored consumer product may be covertures and filling, products based on sugars, breads, dry biscuits, cakes, rice cakes, rice crackers, cookies, crackers, donuts, muffins, pastries, pre-mixes, filings, toppings, fruit or flavored yoghurts, ice creams, fruit ices, frozen desserts, spreads, regular or low fat margarines, butter/margarine blends, flavored oils, shortenings, dressings, spice preparations, peanut butters, fresh cheeses, soft cheeses, milk drinks, wheys, butters, partially or wholly hydrolysed milk protein-containing products, fermented milk products, condensed milk and analogues, gelatins, puddings, dessert creams, chocolates, spreads, aqueous beverages, enhanced/slightly sweetened water drinks, flavored carbonated and still mineral and table waters, carbonated soft drinks, non-carbonated beverages, carbonated waters, still waters, softs, bottled waters, sports/energy drinks, juice drinks, vegetable juices, vegetable juice preparations, beer and malt beverages, spirituous beverages, wines, liquors, instant vegetable drinks, powdered soft drinks, instant coffees and teas, black teas, green teas, oolong teas, herbal infusions, cacaos, tea-based drinks, coffee-based drinks, cacao-based drinks, infusions, syrups, chewing gums, hard and soft candies, frozen fruits, frozen fruit juices, water-based ices, fruit ices, sorbets, breakfast cereals, cereal bars, energy bars/nutritional bars, granolas, pre-cooked ready-made rice products, rice flour products, millet and sorghum products, raw or pre-cooked noodles and pasta products.

The proportions in which the invention's composition can be incorporated into the various of the aforementioned products vary within a wide range of values. These values are dependent on the nature of the consumer product to be flavored and on the desired organoleptic effect as well as the nature of the co-ingredients in a given base when the composition according to the invention are mixed with perfuming or flavoring ingredients, solvents or additives commonly used in the art.

For example, in the case of flavored consumer product, typical concentrations are in the order of 0.001 ppm to 40000 ppm, more preferably, 0.005 ppm to 20000 ppm, even more preferably 0.01 ppm to 10000 ppm, of the invention's composition based on the weight of the consumer product into which they are incorporated.

In a preferred embodiment of the invention, the extract is the strawberry extract of the invention as provided herein.

Preferred consumer flavored consumer product including the strawberry extract of the invention include fruit or flavored yoghurts, ice creams, fruit ices, frozen desserts, aqueous beverages, enhanced/slightly sweetened water drinks, flavored carbonated and still mineral and table waters, carbonated soft drinks, non-carbonated beverages, carbonated waters, still waters and softs, confectionary products such as chewy sweets and chewing gum, and other related products.

The present invention is best illustrated but is not limited to the following examples, wherein the abbreviations have the usual meaning in the art.

### EXAMPLES

### Example 1: Extracting Essential Oils from Jasminium grandiflorum

Referring to FIG. 3, freshly isolated flowers from Jasminium grandiflorum were introduced into a microwave extractor at a rate of 4 Kg per hour. The length of the microwave extractor was 2.5 m. The freshly isolated flowers were subjected to microwave energy at a power of 200 Watts (a power of 0.56 KWH per Kg biomass), to obtain a distillate, while the temperature of the chamber within the microwave extractor (referring to FIGS. 1 and 2 for example), was maintained at a temperature between 60 and 80 degrees Celsius. The transit time of the plant biomass through the microwave extractor was approximately 20 minutes.

The distillate was collected at the bottom of the microwave extractor by gravimetry, and the distillate was applied to a XAD4 resin column. Once the resin in the column became saturated, materials bound to the resin column were desorbed, using ethanol, with a flow rate of approximately 5 bed volumes per hour, wherein the total volume was adjusted to result in a composition comprising essential oils having the desired concentration in ethanol. Here, approximately 3 to 15 bed volumes were required to elute the essential oil off the resin column.

In some experiments, the distillate collected from the microwave extractor, prior to application to the XAD4 resin column comprised essential oils having a concentration from 200 to 3000 ppm. The composition of the essential oils was analyzed by GC, and the results are reported in Table 1 of Example 2 below.

### Example 2: Essential Oils from Jasminium grandiflorum

The results of the CG analysis of three compositions comprising distillate collected from the microwave extractor, prior to application to the XAD4 resin column from three separate experiments are reported in Table 1 below. For comparison, the constituents of essential oils from four commercial sources are included. The sensory properties of the essential oils were also evaluated.

TABLE 1

| | | | | Commercial | Commercial | Commercial | Commercial |
|---|---|---|---|---|---|---|---|
| | Microwave | Microwave | Microwave | Jasmin | Jasmin | Jasmin | Jasmin |
| | Extract 1 | Extract 2 | Extract 3 | Extract 1 | Extract 1 | Extract 3 | Extract 4 |
| | % w/w | % w/w | % w/w | % w/w | % w/w | % w/w | % w/w |
| Compound | GC | GC | GC | GC | GC | GC | GC |
| BENZYL ALCOHOL | 0.20 | 0.63 | 0.67 | 3.25 | 1.76 | 1.47 | 1.55 |
| PHENYLACETALDEHYDE | tr | tr | 0.30 | tr | tr | tr | tr |
| p-CRESOL | 0.10 | 0.13 | 0.25 | 0.21 | 0.15 | 0.42 | 2.19 |
| METHYL BENZOATE | nd | nd | tr | 0.14 | nd | tr | 0.25 |
| LINALOOL | 12.98 | 11.77 | 16.92 | 4.02 | 1.76 | 3.84 | 3.59 |
| BENZYL CYANIDE | 0.11 | tr | tr | tr | nd | nd | nd |
| BENZYL ACETATE | 41.89 | 37.17 | 61.58 | 11.63 | 5.87 | 11.36 | 16.08 |
| a-TERPINEOL | tr | tr | 1.40 | tr | tr | tr | tr |
| NEROL | tr | tr | 0.19 | tr | tr | tr | tr |
| GERANIOL | tr | tr | 0.65 | tr | tr | tr | tr |
| INDOLE | 6.48 | 2.78 | 1.17 | tr | tr | 0.55 | 3.40 |
| METHYL | 0.93 | 0.74 | 0.46 | nd | nd | nd | nd |
| ANTHRANILATE | | | | | | | |
| EUGENOL | 0.45 | 0.51 | 0.61 | 1.03 | 0.32 | 1.48 | 1.60 |
| (Z)-JASMONE | 10.23 | 5.45 | 7.69 | 1.61 | 2.91 | 1.54 | 6.45 |
| JASMIN LACTONE | 1.12 | 0.64 | tr | 0.48 | 0.83 | 0.69 | 1.76 |
| (E,E)-FARNESENE | 5.28 | 6.02 | 1.29 | 1.87 | 1.81 | 0.79 | 2.26 |
| (Z)-3-HEXENYL | 2.01 | 3.22 | 0.68 | 0.85 | 1.20 | 0.77 | 1.25 |
| BENZOATE | | | | | | | |
| (E)-NEROLIDOL | 0.17 | 0.24 | tr | tr | 0.21 | 0.11 | 0.35 |
| METHYL N-ACETYL | 0.21 | 0.14 | tr | 0.34 | 0.80 | tr | tr |
| ANTHRANILATE | | | | | | | |
| (E,E)-4,8,12- | 0.21 | 0.21 | tr | tr | tr | tr | tr |
| TRIMETHYL- | | | | | | | |
| 1,3,7,11- | | | | | | | |
| TRIDECATETRAENE | | | | | | | |
| METHYL (Z)- | 0.24 | 0.19 | tr | 0.80 | 0.67 | 0.35 | 1.01 |
| JASMONATE | | | | | | | |
| METHYL (E)- | 0.39 | 0.28 | tr | tr | 0.53 | tr | tr |
| EPIJASMONATE | | | | | | | |
| JASMIN KETOLACTONE | 0.11 | tr | nd | 0.31 | 0.64 | 0.27 | tr |
| BENZYL BENZOATE | 3.26 | 7.16 | 1.40 | 6.45 | 6.76 | 12.45 | 12.44 |
| HEXAHYDROFARNESYL | 0.84 | 1.78 | 0.32 | 1.54 | 1.12 | tr | tr |
| ACETONE | | | | | | | |
| NEOPHYTADIENE | tr | tr | tr | 0.56 | 1.16 | 1.13 | 0.33 |
| NEOPHYTADIENE | 0.11 | 0.19 | tr | 0.28 | 0.53 | 0.41 | 0.25 |
| ISOMER (T) | | | | | | | |
| METHYL PALMITATE | 0.81 | 1.50 | 0.23 | 0.69 | 1.67 | 0.86 | 1.27 |
| ISOPHYTOL | 3.86 | 5.91 | 1.02 | 9.60 | 10.15 | 5.41 | 6.88 |
| GERANYL LINALOOL | 1.05 | 1.31 | 0.21 | 4.08 | 4.72 | 1.96 | 1.80 |
| METHYL LINOLEATE | tr | tr | nd | 0.13 | 0.28 | 0.15 | 0.18 |
| METHYL LINOLENATE | 0.41 | 0.53 | tr | 1.39 | 2.46 | 1.23 | 3.27 |
| METHYL OLEATE | 0.16 | 0.27 | tr | 0.31 | 0.71 | 0.48 | 0.46 |
| (E)-PHYTOL | 0.96 | 1.19 | tr | 10.76 | 9.79 | 5.19 | 7.73 |
| METHYL STEARATE | tr | tr | nd | 3.44 | 0.55 | 1.77 | 1.31 |
| LINOLENIC ACID | 0.91 | 1.67 | 0.24 | 7.91 | 6.25 | 4.30 | 3.73 |
| (E)-PHYTYL ACETATE | | | | | | | |
| (E)-PHYTYL BENZOATE | nd | nd | nd | 0.76 | 0.71 | 0.45 | 0.47 |
| SQUALENE | tr | tr | nd | 3.41 | 6.30 | 3.74 | 3.52 |
| SQUALENE EPOXIDE | tr | tr | nd | 2.78 | 3.83 | 8.23 | 5.11 |

These data suggest that the methods disclosed herein are capable of reproducibly producing an essential oil from Jasminium grandiflorum comprising from 7 to 11% w/w (z)-jasmone, from 10 to 17% w/w (±)-3,7-Dimethyl-1,6-octadien-3-ol, from 40 to 62% w/w benzyl acetate, and from 1 to 7.1% w/w benzyl benzoate. By way of comparison, jasmine essential oils obtained from commercial sources comprised from 1.5 to 7% w/w (z)-jasmone, from 1.5 to 5% w/w (±)-3,7-Dimethyl-1,6-octadien-3-ol, from 6 to 17% w/w benzyl acetate, and from 6 to 13% w/w benzyl benzoate.

The three compositions comprising distillate collected from the microwave extractor, prior to application to the XAD4 resin column also smelled more like freshly isolated jasmine flowers than the essential oils from commercial sources.

### Example 3: Extracting Essential Oils from Cananga odorata

Freshly isolated flowers from Cananga odorata are introduced into a microwave extractor at a rate of 4 Kg per hour. The length of the microwave extractor is 2.5 m. The freshly isolated flowers are subjected to microwave energy at a power of 200 Watts, to obtain a distillate, while the temperature of the chamber within the microwave extractor (referring to FIGS. 1 and 2 for example), is maintained at a temperature between 60 and 80 degrees Celsius. The transit time of the plant biomass through the microwave extractor is approximately 20 minutes.

The distillate is collected at the bottom of the microwave extractor by gravimetry, and the distillate is applied to a XAD4 resin column. Once the resin in the column becomes saturated, materials bound to the resin column are desorbed, using ethanol, with a flow rate of approximately 5 bed volumes per hour, wherein the total volume is adjusted to result in a composition comprising essential oils having the desired concentration in ethanol. Here, approximately 3 to 15 bed volumes may be required to elute the essential oil off the resin column.

In some experiments, the distillate that may be collected from the microwave extractor, prior to application to the XAD4 resin column may comprise essential oils having a concentration from 200 to 3000 ppm. The composition of the essential oils will be analyzed by GC.

### Example 4: Extracts Prepared from Piper nigrum by a Method According to the Aspects Presented Herein

Dried peppercorns of the Piper nigrum plant were ground, and introduced into a microwave extractor at a rate of 8 Kg per hour. The length of the microwave extractor was 2.5 m. The ground peppercorns were subjected to microwave energy at a power of 0.27 KWH per kilogram of biomass, to obtain a distillate, while the temperature of the chamber within the microwave extractor (referring to FIGS. 1 and 2 for example), was maintained at a temperature between 81 and 100 degrees Celsius. Steam was also added to the extractor at a rate of 8 Kg/hour. The transit time of the biomass through the microwave extractor was approximately 20 minutes.

The distillate was collected at the bottom of the microwave extractor by gravimetry, and the distillate was applied to a XAD4 resin column. Once the resin in the column became saturated, materials bound to the resin column were desorbed, using ethanol, with a flow rate of approximately 5 bed volumes per hour, wherein the total volume was adjusted to result in a composition comprising extracts having the desired concentration in ethanol. Here, approximately 3 to 15 bed volumes were required to elute the extract off the resin column. The composition of the ethanolic extract (Microwave Extract 1) and the distillate (Microwave Extract 2) was analyzed by GC, and the results are reported in Table 2 below.

TABLE 2

| | Microwave | Microwave | Commercial | Commercial | Commercial |
|---|---|---|---|---|---|
| | Extract 1 | Extract 2 | Extract 1 | Extract 2 | Extract 3 |
| | % w/w | % w/w | % w/w | % w/w | % w/w |
| Compound | GC | GC | GC | GC | GC |
| α-THUJENE | 1.67 | 0.77 | tr | 1.11 | tr |
| α-PINENE | 8.95 | 5.70 | tr | 5.99 | 0.68 |
| CAMPHENE | 0.17 | 0.14 | nd | 0.14 | tr |
| SABINENE | 14.31 | 9.43 | 0.69 | 6.67 | tr |
| β-PINENE | 15.75 | 12.41 | 0.64 | 9.19 | 2.28 |
| MYRCENE | 2.77 | 1.38 | 0.36 | 1.69 | 0.57 |
| α-PHELLANDRENE | 1.66 | tr | 0.31 | 2.17 | 0.41 |
| δ3-CARENE | 10.72 | 6.62 | 1.30 | 10.50 | 3.35 |
| α-TERPINENE | tr | tr | tr | 0.48 | tr |
| p-CYMENE | 0.73 | 1.16 | 0.16 | 0.75 | 0.54 |
| β-PHELLANDRENE | Coelut° | Coelut° | 0.61 | 1.52 | Coelut° |
| | (≈0.7%) | (≈0.5%) | | | |
| LIMONENE | 27.91 | 20.56 | 6.84 | 15.29 | 7.97 |
| (E)-b-OCIMENE | 0.18 | tr | 0.11 | 0.12 | tr |
| γ-TERPINENE | 0.10 | tr | tr | 0.78 | tr |
| (E)-SABINENE HYDRATE | 0.16 | 0.23 | 0.28 | tr | tr |
| ISOSYLVETERPINOLENE | 0.12 | tr | tr | tr | tr |
| TERPINOLENE | 0.39 | tr | 0.28 | 0.61 | tr |
| (Z)-SABINENE HYDRATE | tr | 0.23 | 0.23 | tr | tr |
| LINALOOL | 0.11 | 0.23 | 0.24 | 0.44 | tr |
| 4-TERPINEOL | 0.15 | 0.71 | 0.36 | 0.98 | 0.15 |
| α-TERPINEOL | tr | 0.28 | 0.29 | tr | tr |
| 2-METHYL-5-(2- | tr | 0.50 | tr | tr | tr |
| PROPANYL)-3- | | | | | |
| CYCLOHEXENE-1,2-DIOL | | | | | |
| ISOMER | | | | | |
| δ-ELEMENE | 0.62 | 0.60 | 3.78 | 2.16 | 1.42 |
| α-CUBEBENE | 0.10 | 0.15 | 0.18 | 0.24 | tr |
| α-COPAENE | 0.17 | 0.31 | 0.37 | 2.41 | tr |
| β-ELEMENE | 0.32 | 0.63 | 1.15 | 1.18 | 1.10 |
| α-GURJUNENE | 0.11 | 0.15 | 0.27 | 0.18 | tr |
| β-CARYOPHYLLENE | 7.64 | 11.58 | 22.23 | 22.31 | 32.38 |
| α-GUAIENE | 0.25 | 0.43 | 0.70 | 0.28 | 0.57 |
| α-HUMULENE | 0.48 | 1.02 | 1.64 | 1.48 | 1.90 |
| GERMACRENE D | 0.22 | 0.14 | 1.16 | 0.50 | 1.67 |
| β-SELINENE | 0.90 | 2.74 | 3.43 | 1.45 | 1.42 |
| α-SELINENE | 0.81 | 1.51 | 3.45 | 1.16 | 1.06 |
| β-BISABOLENE | tr | tr | tr | 0.86 | tr |
| δ-CADINENE | tr | tr | tr | 0.87 | tr |
| ELEMOL | tr | tr | tr | 0.45 | tr |
| SPATHULENOL | 0.15 | 1.53 | 0.90 | tr | tr |
| CARYOPHYLLENE OXIDE | 0.31 | 5.26 | 0.92 | 0.61 | 1.45 |
| (2 ISOMERES) | | | | | |
| HUMULENE OXIDE | tr | 0.38 | 0.12 | tr | tr |
| ISOSPATHULENOL | 0.26 | 1.40 | 1.24 | 0.14 | 1.20 |
| PELLITORIN | nd | nd | 0.94 | nd | 2.67 |
| PALMITIC ACID | nd | nd | 0.35 | nd | 0.15 |
| 1-CINNAMOYL PIPERIDINE | nd | nd | 0.31 | nd | 0.10 |
| SARMENTINE (=1-(1- | nd | nd | 0.23 | nd | 0.23 |
| PYRROLIDINYL)-(E,E)-2,4- | | | | | |
| DECADIEN-1-ONE) | | | | | |
| PHYTOL | nd | nd | 0.22 | nd | nd |
| NEOPELLITORIN B | nd | nd | 0.24 | nd | 0.22 |
| LINOLEIC ACID | nd | nd | 1.28 | nd | 0.55 |
| N-ISOBUTYL-DODECA-2,4- | nd | nd | | nd | |
| DIENAMIDE | | | | | |
| OLEIC ACID | nd | nd | 0.26 | nd | tr |
| 1-DODECANOYL | nd | nd | 0.20 | nd | tr |
| PIPERIDINE | | | | | |
| N-(2,4- | nd | nd | 0.32 | nd | 0.17 |
| DODECADIENOYL)PYRROLIDINE | | | | | |
| N-(4′,4′- | nd | nd | 1.54 | nd | 1.86 |
| DIHYDROPIPEROYL)PIPERIDINE | | | | | |
| AMIDE MW = 335 G/MOL (T) | nd | nd | 0.77 | nd | 1.63 |
| AMIDE MW = 333 G/MOL (T) | nd | nd | 3.16 | nd | 0.63 |
| AMIDE MW = 335 G/MOL (T) | nd | nd | 1.57 | nd | 0.33 |
| PIPERINE | nd | nd | 3.57 | nd | 3.20 |
| PIPERINE DERIV. | nd | nd | 2.21 | nd | 1.09 |
| MW = 315 G/MOL (T) | | | | | |
| AMIDE MW = 329 G/MOL (T) | nd | nd | 0.87 | nd | 0.67 |
| PIPERIDINE DERIV. | nd | nd | 3.89 | nd | 3.39 |
| MW = 343 G/MOL (T) | | | | | |

These data suggest that the methods disclosed herein are capable of reproducibly producing an essential oil from Piper nigrum comprising from 9 to 15% w/w sabinine, from 12 to 16% w/w β-pinene, from 6 to 11% w/w δ3-carene, and from 20 to 30% w/w limonene. Furthermore, amount of beta-caryophyllene in an essential oil from Piper nigrum produced using the methods disclosed herein was from 7 to 12% w/w, and piperine was not detected.

By way of comparison, an essential oil from Piper nigrum obtained from a commercial source comprised approximately 3% w/w piperine, from 20 to 35% w/w beta-caryophyllene, from 0.5 to 7% w/w sabinine, from 0.5 to 10% w/w β-pinene, from 1 to 11% w/w δ3-carene, and from 6 to 16% w/w limonene.

### Example 5: Extracts Prepared from Pears by a Method According to the Aspects Presented Herein

Fresh pear fruits were sliced, and introduced into a microwave extractor at a rate of 7 Kg per hour. The length of the microwave extractor was 2.5 m. The sliced pears were subjected to 200 W microwave energy at a power of 0.33 KWH per kilogram of biomass, to obtain a distillate, while the temperature of the chamber within the microwave extractor (referring to FIGS. 1 and 2 for example), was maintained at a temperature between 60 and 80 degrees Celsius. The transit time of the biomass through the microwave extractor was approximately 20 minutes.

The distillate was collected at the bottom of the microwave extractor by gravimetry, and the distillate was applied to a XAD4 resin column. Once the resin in the column became saturated, materials bound to the resin column were desorbed, using ethanol, with a flow rate of approximately 5 bed volumes per hour, wherein the total volume was adjusted to result in a composition comprising extract having the desired concentration in ethanol. Here, approximately 3 to 15 bed volumes were required to elute the extract off the resin column. The composition of the ethanolic extract (Microwave Extract 1) and the distillate (Microwave Extract 2) was analyzed by GC, and the results are reported in Table 3 below.

TABLE 3

| | Micro- | Micro- | Commer- |
|---|---|---|---|
| | wave | wave | cial |
| | Extract 1 | Extract 2 | Extract |
| Compound | % w/w GC | % w/w GC | % w/w GC |
| 1-PROPANOL | 2.2 | 0.1 | 16.7 |
| BUTYL ACETATE | 11.0 | 0.3 | 2.2 |
| HEXANAL | nd | nd | 1.3 |
| ISOBUTYL ALCOHOL | 1.4 | 0.0 | 8.7 |
| 1-BUTANOL | 23.4 | 0.7 | 33.1 |
| (E)-2-HEXENAL | nd | nd | 3.4 |
| PENTYL ACETATE | 0.3 | nd | nd |
| 2-METHYLBUTYL | 0.6 | nd | 6.7 |
| ALCOHOL | | | |
| 3-METHYLBUTYL | nd | nd | 3.7 |
| ALCOHOL | | | |
| 1-PENTANOL | 0.1 | nd | 0.6 |
| HEXYL ACETATE | 6.2 | 0.1 | nd |
| ACETOIN | 1.5 | nd | 2.2 |
| (=ACETYLMETHYL | | | |
| CARBINOL) | | | |
| ACETOL | 0.9 | 3.2 | nd |
| 1-HEXANOL | 2.5 | 0.1 | 13.3 |
| (Z)-3-HEXENOL | nd | nd | 0.4 |
| 1-HYDROXY-3-BUTEN- | 0.6 | nd | nd |
| 2-ONE | | | |
| 2-OXO-3-BUTENOL (T) | nd | 3.1 | nd |
| (E)-2-HEXENOL | nd | nd | 2.5 |
| ACETIC ACID | 2.1 | 7.5 | nd |
| FURFURAL | 1.5 | 0.6 | 0.4 |
| 2-ETHYL-1-HEXANOL | 0.3 | nd | nd |
| BENZALDEHYDE | nd | nd | 0.2 |
| 4-CYCLOPENTENE-1,3- | nd | 0.2 | nd |
| DIONE | | | |
| 3- | 0.1 | nd | nd |
| (METHYLTHIO)PROPYL | | | |
| ACETATE | | | |
| FURFURYL ALCOHOL | 0.6 | 1.4 | nd |
| 2-METHYLBUTYRIC | 1.4 | 0.4 | nd |
| ACID | | | |
| 3- | 0.2 | nd | nd |
| (METHYLTHIO)PROPYL | | | |
| ALCOHOL | | | |
| CRATONE (=2(5H)- | nd | 0.3 | nd |
| FURANONE) | | | |
| 2-HYDROXY-2- | nd | 1.5 | nd |
| CYCLOPENTENONE | | | |
| CITRONELLOL | 1.3 | nd | nd |
| METHYL 2,4- | 0.9 | nd | nd |
| DECADIENOATE | | | |
| NEROL | 0.5 | nd | nd |
| ETHYL 2,4- | 0.7 | nd | nd |
| DECADIENOATE | | | |
| QUINCESTER (=ETHYL | 0.1 | nd | nd |
| (E,Z,Z)-2,4,7- | | | |
| DECATRIENOATE) | | | |
| GERANIOL | 1.6 | nd | nd |
| METHYL 3- | 1.3 | nd | 0.1 |
| HYDROXYOCTANOATE | | | |
| HEXANOIC ACID | nd | 0.9 | nd |
| ETHYL 3- | 3.7 | 0.1 | 0.3 |
| HYDROXYOCTANOATE | | | |
| 2-PHENYLETHYL | 1.4 | nd | nd |
| ALCOHOL | | | |
| METHYL (Z)-3- | 0.1 | nd | nd |
| HYDROXY-5- | | | |
| OCTENOATE | | | |
| ETHYL (Z)-3- | 0.2 | nd | nd |
| HYDROXY-5- | | | |
| OCTENOATE | | | |
| 1,3- | 6.1 | 45.6 | nd |
| DIHYDROXYACETONE | | | |
| ISOMER (T) | | | |
| 1,3-OCTANEDIOL | 6.8 | 0.3 | nd |
| P-VINYL GUAIACOL | 0.1 | nd | nd |
| DIHYDROMYRCENOL | 0.2 | nd | nd |
| 5-HYDROXY-5,6- | 0.8 | 3.6 | nd |
| DIHYDROMALTOL | | | |
| P-VINYL PHENOL | 0.4 | nd | nd |
| 5-HYDROXYMETHYL | 1.2 | 4.7 | nd |
| FURFURAL | | | |
| 5-HYDROXYMETHYL | 0.1 | 1.3 | nd |
| FURFUROL | | | |

These data suggest that the methods disclosed herein are capable of reproducibly producing an extract from pears comprising from 0.2 to 12% w/w butyl acetate, from 0.1 to 6% w/w hexyl acetate, from 3 to 15% w/w acetol, from 2 to 8% w/w acetic acid, from 0.5 to 2% w/w furfural, from 0.5 to 2% w/w furfural alcohol, from 0.2 to 2% w/w 2-methylbutyric acid, from 0.1 to 4% w/w ethyl 3-hydroxyoctanoate, from 6 to 46% w/w 1,3-dihydroxyacetone, from 0.2 to 7% w/w 1,3-octanediol, from 0.5 to 4% w/w 5-hydroxy-5,6-dihydrolmaltol, and from 1 to 4% w/w 5-hydroxymethyl furfural. In addition, in the ethanolic extract, methyl 2,4-decadienoate, ethyl 2,4-decadienoate, geraniol, methyl 3-hydroxyoctanoate, 2-phenylethyl alcohol, and p-vinyl phenol were also present.

By way of comparison, a pear extract obtained from a commercial source comprised approximately 2% w/w butyl acetate, approximately 0.4% w/w furfural, and approximately 0.3% w/w ethyl 3-hydroxyoctanoate. In addition, hexyl acetate, acetol, acetic acid, furfural alcohol, 2-methylbutyric acid, 1,3-dihydroxyacetone, 1,3-octanediol, methyl 2,4-decadienoate, ethyl 2,4-decadienoate, 5-hydroxy-5,6-dihydrolmaltol, geraniol, methyl 3-hydroxyoctanoate, 2-phenylethyl alcohol, 5-hydroxymethyl furfural and p-vinyl phenol were not detected.

The extract from pears produced according to the methods described herein was very fruity and had a juicy, delicious and gourmand note of William pear and was very much powerful than the commercial extract.

### Example 6: Extracts Prepared from Green Tea Leaves by a Method According to the Aspects Presented Herein

Dried green tea leaves were hydrated by the addition of water to result in a biomass having a weight of 200% of the dried biomass, and introduced into a microwave extractor at a rate of 10 Kg per hour. The length of the microwave extractor was 2.5 m. The hydrated leaves were subjected to 300 W microwave energy at a power of 0.43 KWH per kilogram of biomass, to obtain a distillate, while the temperature of the chamber within the microwave extractor (referring to FIGS. 1 and 2 for example), was maintained at a temperature between 70 and 90 degrees Celsius. The transit time of the biomass through the microwave extractor was approximately 20 minutes.

The distillate was collected at the bottom of the microwave extractor by gravimetry, and the distillate was applied to a XAD4 resin column. Once the resin in the column became saturated, materials bound to the resin column were desorbed, using ethanol, with a flow rate of approximately 5 bed volumes per hour, wherein the total volume was adjusted to result in a composition comprising an extract having the desired concentration in ethanol. Here, approximately 3 to 15 bed volumes were required to elute the essential oil of the resin column. The composition of the ethanolic extract (Microwave Extract 1) and the distillate (Microwave Extract 2) was analyzed by GC, and the results are reported in Table 4 below.

TABLE 4

| | Micro- | Micro- | Commer- |
|---|---|---|---|
| | wave | wave | cial |
| | Extract 1 | Extract 2 | Extract |
| Compound | % w/w GC | % w/w GC | % w/w GC |
| 1-PENTEN-3-OL | 11.7 | 1.1 | 0.9 |
| 3-METHYLBUTYL | 2.5 | 0.4 | 0.8 |
| ALCOHOL | | | |
| ACETYLMETHYL | 1.0 | nd | 1.1 |
| CARBINOL (=3- | | | |
| HYDROXY-2-BUTANONE) | | | |
| ACETOL (=1-HYDROXY- | 1.1 | 2.7 | 14.8 |
| 2-PROPANONE) | | | |
| (Z)-2-PENTENOL | 6.9 | 1.0 | 0.5 |
| (Z)-3-HEXENOL | 2.8 | nd | nd |
| (Z)-LINALOOL OXIDE | 0.8 | nd | nd |
| (FURANOID) | | | |
| ACETIC ACID | 1.0 | 16.0 | 33.7 |
| (E)-LINALOOL OXIDE | 1.4 | 0.2 | nd |
| (FURANOID) | | | |
| BENZALDEHYDE | 1.0 | nd | nd |
| 3,5-OCTADIEN-2-ONE | 1.0 | nd | nd |
| ISOMER (T) | | | |
| LINALOOL | 4.1 | 0.5 | 1.6 |
| 3,5-OCTADIEN-2-ONE | 0.9 | nd | nd |
| ISOMER (T) | | | |
| 2-HYDROXY-2,6,6- | 3.2 | nd | nd |
| TRIMETHYL-1- | | | |
| CYCLOHEXONE | | | |
| FURFURYL ALCOHOL | 1.2 | nd | 8.2 |
| GERANIOL | 5.0 | 0.4 | 1.4 |
| BENZYL ALCOHOL | 5.0 | 1.0 | 1.3 |
| 2-PHENYLETHYL | 3.5 | 0.7 | 1.6 |
| ALCOHOL | | | |
| b-IONONE | 0.8 | nd | nd |
| 2-ACETYL PYRROLE | 2.7 | 0.7 | 3.7 |
| 4,5-EPOXY-b-IONONE | 0.8 | nd | nd |
| PHENOL | 1.4 | 0.6 | 1.0 |
| (Z)-JASMONE | 0.6 | nd | nd |
| DIHYDROACTINIDIOLIDE | 1.3 | 2.3 | 2.1 |
| (=CASSIA LACTONE) | | | |
| 4-VINYL PHENOL | 0.8 | 0.7 | 2.8 |
| CAFFEINE | 12.2 | 28.6 | 1.9 |

These data suggest that the methods disclosed herein are capable of reproducibly producing an extract from green tea leaves comprising from 12 to 30% w/w caffeine, and approximately 1% w/w furfuryl alcohol. In addition, 3,5-octadien-2-one, β-ionone, and 4,5-epoxy-β-ionone were also present.

By way of comparison, a green tea extract from a commercial source comprised approximately 2% w/w caffeine, and approximately 8% w/w furfural alcohol. In addition, 3,5-octadien-2-one, β-ionone, and 4,5-epoxy-β-ionone were absent.

### Example 7: Extracts Prepared from Ginger Roots by a Method According to the Aspects Presented Herein

Fresh ginger roots were sliced, and introduced into a microwave extractor at a rate of 7 Kg per hour. The length of the microwave extractor was 2.5 m. The sliced roots were subjected to 300 W microwave energy at a power of 0.45 KWH per kilogram of biomass, to obtain a distillate, while the temperature of the chamber within the microwave extractor (referring to FIGS. 1 and 2 for example), was maintained at a temperature between 70 and 90 degrees Celsius. The transit time of the biomass through the microwave extractor was approximately 20 minutes.

The distillate was collected at the bottom of the microwave extractor by gravimetry, and the distillate was applied to a XAD4 resin column. Once the resin in the column became saturated, materials bound to the resin column were desorbed, using ethanol, with a flow rate of approximately 5 bed volumes per hour, wherein the total volume was adjusted to result in a composition comprising an extract having the desired concentration in ethanol. Here, approximately 3 to 15 bed volumes were required to elute the essential oil of the resin column. The composition of the ethanolic extract (Microwave Extract 1) and the distillate (Microwave Extract 2) was analyzed by GC, and the results are reported in Table 5 below.

TABLE 5

| | Micro- | Micro- | Commer- |
|---|---|---|---|
| | wave | wave | cial |
| | Extract 1 | Extract 2 | Extract |
| Compound | % w/w GC | % w/w GC | % w/w GC |
| α-PINENE | nd | nd | 1.5 |
| 2-METHYL-3-BUTEN-2-OL | 0.2 | nd | <0.1 |
| CAMPHENE | 0.0 | nd | 4.5 |
| β-PINENE | nd | nd | 0.2 |
| HEXANAL | 0.1 | nd | <0.1 |
| α-PHELLANDRENE | nd | nd | 0.2 |
| MYRCENE | nd | nd | 1.0 |
| 2-HEPTANONE | 0.4 | nd | <0.1 |
| LIMONENE | nd | nd | 1.0 |
| β-PHELLANDRENE | nd | nd | 4.4 |
| EUCALYPTOL | 14.8 | 13.9 | 2.9 |
| TERPINOLENE | nd | nd | 0.2 |
| OCTANAL | 0.0 | nd | <0.1 |
| 2-HEPTANOL | 5.7 | 6.6 | 0.6 |
| 6-METHYL-5-HEPTEN-2- | 2.1 | nd | 0.3 |
| ONE | | | |
| 2-NONANONE | 0.3 | nd | 0.2 |
| (E)-2-OCTENAL | 0.1 | nd | <0.1 |
| CITRONELLAL | 0.6 | 0.3 | 0.6 |
| α-COPAENE | nd | nd | 0.3 |
| CAMPHOR | 0.3 | nd | <0.1 |
| 2-NONANOL | 0.9 | 0.7 | 0.4 |
| (Z)-ISOCITRAL | 0.3 | nd | <0.1 |
| LINALOOL | 2.2 | 2.0 | 0.6 |
| (E)-ISOCITRAL | 0.6 | nd | <0.1 |
| β-ELEMENE | nd | nd | 0.4 |
| 2-UNDECANONE | nd | nd | 0.7 |
| 4-TERPINEOL | 0.6 | 0.6 | 0.2 |
| OXYGENATED TERPENE | 0.5 | nd | <0.1 |
| MW = 152 G/MOL (T) | | | |
| NERAL | 19.7 | 25.0 | 8.8 |
| α-TERPINEOL | 3.7 | 3.5 | 0.9 |
| BORNEOL | 3.7 | 3.7 | 1.8 |
| γ-CADINENE | nd | nd | 2.0 |
| ZINGIBERENE | nd | nd | 18.5 |
| GERANIAL | 26.1 | 29.0 | 16.3 |
| OXYGENATED TERPENE | 0.9 | nd | <0.1 |
| MW = 152 G/MOL (T) | | | |
| (E,E)-α-FARNESENE | nd | nd | 5.4 |
| β- | nd | nd | 6.9 |
| SESQUIPHELLANDRENE | | | |
| AR-CURCUMENE | nd | nd | 3.3 |
| CITRONELLOL | 2.3 | 2.0 | 3.3 |
| MYRTENOL | 0.2 | nd | <0.1 |
| NEROL | 0.7 | 0.7 | 0.2 |
| GERMACRENE B | nd | nd | 0.5 |
| TERPINOLEN-6-OL (T) | 0.5 | nd | <0.1 |
| GERANIOL | 3.0 | 3.0 | 1.0 |
| (E)-NEROLIDOL | nd | nd | 0.5 |
| ELEMOL | nd | nd | 0.3 |
| ZINGIBERENOL | nd | nd | 0.6 |
| (E)-SESQUIPIPERITOL | nd | nd | 0.4 |

These data suggest that the methods disclosed herein are capable of reproducibly producing an essential oil from ginger roots comprising from 13 to 15% w/w eucalyptol, from 2 to 6% w/w 6-methyl-5-hepten-2-one, from 19 to 25% w/w neral, approximately 0.4% w/w 2-heptanone, from 26 to 29% w/w geranial, approximately 0.7% w/w nerol, and approximately 0.3% w/w geraniol. Furthermore, zingiberene was not detected.

By way of comparison, an ginger essential oil obtained from a commercial source comprised approximately 0.1% w/w 2-heptanone, approximately 2.9% w/w eucalyptol, approximately 0.3% w/w 6-methyl-5-hepten-2-one, approximately 8.8% w/w neral, approximately 16.3% w/w geranial, approximately 18.5% w/w zingiberene, approximately 0.2% w/w nerol, and approximately 1% w/w geraniol.

### Example 8: Extracts Prepared from Coffee Beans by a Method According to the Aspects Presented Herein

Freshly roased coffee beans were ground, and introduced into a microwave extractor at a rate of 12 Kg per hour. The length of the microwave extractor was 2.5 m. The ground beans were subjected to 120 W microwave energy at a power of 0.24 KWH per kilogram of biomass, to obtain a distillate, while the temperature of the chamber within the microwave extractor (referring to FIGS. 1 and 2 for example), was maintained at a temperature between 70 and 90 degrees Celsius. The transit time of the biomass through the microwave extractor was approximately 20 minutes.

The distillate was collected at the bottom of the microwave extractor by gravimetry, and the distillate was applied to a XAD4 resin column. Once the resin in the column became saturated, materials bound to the resin column were desorbed, using ethanol, with a flow rate of approximately 5 bed volumes per hour, wherein the total volume was adjusted to result in a composition comprising essential oils having the desired concentration in ethanol. Here, approximately 3 to 15 bed volumes were required to elute the essential oil of the resin column. The composition of the ethanolic extract (Microwave Extract 1) and the distillate (Microwave Extract 2) was analyzed by GC, and the results are reported in Table 6 below.

TABLE 6

| | Micro- | Micro- | Commer- |
|---|---|---|---|
| | wave | wave | cial |
| | Extract 1¹ | Extract 2 | Extract² |
| Compound | % w/w GC | % w/w GC | % w/w GC |
| 2,3-PENTANEDIONE | 0.6 | 0.6 | nd |
| CYCLOPENTANONE | 0.1 | nd | nd |
| PYRIDINE | 2.3 | 6.7 | 0.4 |
| PYRAZINE | 0.6 | 0.6 | nd |
| 2-FURFURYL METHYL | 0.1 | nd | nd |
| ETHER | | | |
| ISOPRENOL | 0.2 | nd | nd |
| COFFEE FURANONE (=2- | 2.3 | 2.0 | 0.1 |
| METHYLTETRAHYDRO- | | | |
| FURAN-3-ONE) | | | |
| 2-METHYL PYRAZINE | 3.7 | 3.1 | 0.6 |
| ACETYLMETHYL | 0.7 | 1.7 | nd |
| CARBINOL | | | |
| ACETOL | 0.7 | 7.5 | 0.0 |
| 2,5-DIMETHYL PYRAZINE | 1.1 | 1.1 | 1.2 |
| 2,6-DIMETHYL PYRAZINE | 1.3 | 1.1 | |
| 2-ETHYL PYRAZINE | 0.6 | 0.5 | 0.2 |
| 3-HYDROXY-2- | 0.5 | 0.5 | nd |
| PENTANONE | | | |
| 2,3-DIMETHYL PYRAZINE | 0.2 | 0.2 | 0.1 |
| 2-CYCLOPENTENONE | 0.2 | 0.2 | nd |
| 2-HYDROXY-3- | 0.4 | 0.4 | nd |
| PENTANONE | | | |
| 1-HYDROXY-2- | 0.8 | 1.2 | nd |
| BUTANONE | | | |
| DIACETONE ALCOHOL | nd | nd | nd |
| 2-ETHYL-6-METHYL | 0.4 | 0.4 | 1.6 |
| PYRAZINE | | | |
| 2-ETHYL-5-METHYL | 0.2 | 0.3 | 0.5 |
| PYRAZINE | | | |
| 2-ETHYL-3-METHYL | 0.4 | 0.1 | 0.3 |
| PYRAZINE | | | |
| 3-ETHYL-2,5-DIMETHYL | nd | nd | nd |
| PYRAZINE | | | |
| ACETIC ACID | 5.3 | 5.2 | 10.9 |
| FURFURAL | 3.5 | 4.7 | 0.4 |
| 2-OXOPROPYL ACETATE | 3.5 | 3.2 | nd |
| FURFURYL METHYL | nd | nd | nd |
| SULFIDE | | | |
| FURFURYL FORMATE | 1.5 | 1.4 | 0.1 |
| 2-ACETYL FURAN | | | 0.3 |
| PYRROLE | 0.2 | 0.2 | nd |
| (2-FURYL)-2-PROPANONE | 0.2 | 0.2 | nd |
| 2-OXOPROPYL | 0.5 | 0.4 | 0.3 |
| PROPANOATE | | | |
| 2-OXOBUTYL ACETATE | 0.5 | 2.1 | nd |
| FURFURYL ACETATE | 1.5 | | 2.1 |
| PROPANOIC ACID | 2.5 | 0.9 | 0.8 |
| 5-METHYL FURFURAL | 2.7 | 2.5 | 2.0 |
| 2-FORMYL-5-METHYL | 3.7 | 0.3 | 0.3 |
| PYRROLE | | | |
| g-BUTYROLACTONE | | 3.2 | 3.3 |
| 2-ACETYL-1-METHYL | 0.2 | 0.2 | 0.3 |
| PYRROLE | | | |
| FURFURYL ALCOHOL | 42.3 | 36.6 | 24.3 |
| ISOVALERIC ACID | 2.8 | 1.5 | 1.8 |
| N-FURFURYL PYRROLE | nd | nd | 0.5 |
| NUSSOL (=2-HYDROXY- | 0.4 | 0.3 | 1.2 |
| 3-METHYL-2-CYCLO- | | | |
| PENTEN-1-ONE) | | | |
| GUAIACOL (=2- | 0.3 | 0.2 | 1.4 |
| METHOXYPHENOL) | | | |
| ETHYL | 0.1 | 0.1 | nd |
| CYCLOPENTENOLONE | | | |
| 2-ACETYL PYRROLE | 0.5 | 0.4 | 2.6 |
| DIFURFURYL ETHER | 0.1 | 0.1 | 0.6 |
| PHENOL | 0.3 | 0.4 | 0.7 |
| 2-FORMYL PYRROLE | 0.5 | 0.4 | Coelut° |
| 2-FORMYL-5- | 0.1 | 0.1 | nd |
| METHYLPYRROLE | | | |
| CAPROLACTAM | 0.1 | 0.1 | nd |
| 4-VINYL GUAIACOL | 0.2 | nd | 3.2 |

¹Caffeine was detected at 0.00151 wt %, theobromine was detected at less than 0.004 wt %, and theophylline was detected at less than 0.02 wt %. Chlorogenic acids were not detected.
²Caffeine was detected at 0.00008 wt %, theobromine was detected at less than 0.0005 wt %, and theophylline was detected at less than 0.001 wt %. 4-O-caffeoylquinic acid (crypto-chlorogenic acid or 4-CQA) was detected at less than 0.00007 wt %, 5-feruloylquinic acid was detected at less than 0.0002 wt %. 3-O-feruloylquinic acid was detected at less than 0.00008 wt %. 4-O-feruloylquinic acid was detected at less than 0.00006 wt %.

These data suggest that the methods disclosed herein are capable of reproducibly producing an extract from coffee beans comprising approximately 0.6% w/w 2,3-pentanedione, from 2 to 2.5% w/w coffee furanone, from 3 to 4% w/w 2-methyl pyrazine, from 0.5 to 2% w/w acetylmethyl carbinol, from 0.5 to 8% w/w acetol, approximately 0.2% w/w 2-cyclopentenone, approximately 0.4% w/w 2-hydroxy-3-pentanone, 0.5 to 1.5% w/w 1-hydroxy-2-butanone, 3 to 5% w/w furfural, approximately 1.5% w/w furfuryl formate, 0.5 to 3% w/w propanoic acid, and 35 to 45% w/w furfuryl alcohol.

By way of comparison, extracts from commercial sources comprised 0.1% w/w coffee furanone, 0.6% w/w 2-methyl pyrazine, 0.4% w/w furfural, 0.1% w/w furfuryl formate, 0.8% w/w propanoic acid, 24% w/w furfuryl alcohol. Furthermore, 2,3-pentanedione, acetylmethyl carbinol, acetol, 2-cyclopentenone, 2-hydroxy-3-pentanone, and 1-hydroxy-2-butanone were not detected.

The differences in the coffee extract obtained according to a method according to the aspects presented herein differs from a commercially obtained extract in volatile compounds that contribute to the sweet notes and olfactive profile of fresh coffee.

### Example 9: Essential Oils from Strawberry Extract

The results of the HPLC analysis of three compositions comprising distillate collected from the microwave extractor are reported in Table 7 below. For comparison, the constituents of essential oils from a commercial source were included.

TABLE 7

| | Commercial | Commercial | |
|---|---|---|---|
| | Extract - | Extract - | Microwave |
| | Aqueous | Ethanolic | Extract |
| | % w/w | % w/w | % w/w |
| Compound | HPLC/CI³ | HPLC/CI⁴ | HPLC/CI⁵ |
| TOTAL ANTHOCYANINS | nd | nd | 0.0000919 |
| TOTAL | nd | nd | 0.0000661 |
| ANTHOCYANIDINS | | | |
| TOTAL ANTHOCYANINS | nd | nd | 0.0000158 |
| AND ANTHOCYANIDINS | | | |
| CYANIDIN | nd | nd | nd |
| CYANIDIN-3- | nd | nd | nd |
| ARABINOSIDE | | | |
| CYANIDIN-3- | nd | nd | nd |
| GALACTOSIDE | | | |
| CYANIDIN-3-GLUCOSIDE | nd | nd | <0.00005 |
| DELPHINIDIN | nd | nd | nd |
| DELPHINIDIN-3- | nd | nd | nd |
| ARABINOSIDE | | | |
| DELPHINIDIN-3- | nd | nd | nd |
| GALACTOSIDE | | | |
| DELPHINIDIN-3- | nd | nd | nd |
| GLUCOSIDE | | | |
| MALVINIDIN | nd | nd | nd |
| MALVINIDIN-3- | nd | nd | nd |
| ARABINOSIDE | | | |
| MALVINIDIN-3- | nd | nd | nd |
| GALACTOSIDE | | | |
| MALVINIDIN-3- | nd | nd | nd |
| GLUCOSIDE | | | |
| PEONIDIN | nd | nd | 0.00000649 |
| PEONIDIN-3- | nd | nd | nd |
| ARABINOSIDE | | | |
| PEONIDIN-3- | nd | nd | 0.0000169 |
| GALACTOSIDE | | | |
| PEONIDIN-3-GLUCOSIDE | nd | nd | 0.0000753 |
| PETUNIDIN | nd | nd | 0.0000596 |
| PETUNIDIN-3- | nd | nd | nd |
| ARABINOSIDE | | | |
| PETUNIDIN-3- | nd | nd | nd |
| GALACTOSIDE | | | |
| PETUNIDIN-3- | nd | nd | nd |
| GLUCOSIDE | | | |

³Glucose, fructose, sucrose lactose, maltose, and galactose were present each at less than 0.2 g/100 g of the extract. The total amount of reducing sugars was less than 0.2 g/100 g of the extract. The total amount of mono and disaccharides was less than 0.2 g/100 g of the extract.
⁴Glucose was present at 0.9 ± 0.7 g/100 g of the extract; Fructose was present at 1.1 ± 0.6 g/100 g of the extract; sucrose, lactose, maltose, and galactose were present each at less than 0.2 g/100 g of the extract. The total amount of reducing sugars was 2.0 ± 0.7 g/100 g of the extract. The total amount of mono and disaccharides was 2.0 ± 0.7 g/100 g of the extract.
⁵Glucose was present at 1.1 ± 0.7 g/100 g of the extract; Fructose was present at 1.4 ± 0.6 g/100 g of the extract; sucrose was present at 0.9 ± 0.5 g/100 g of the extract; lactose, maltose, and galactose were present each at less than 0.2 g/100 g of the extract. The total amount of reducing sugars was 2.5 ± 0.8 g/100 g of the extract. The total amount of mono and disaccharides was 3.5 ± 0.9 g/100 g of the extract.

These data suggest that the methods presented herein provide a strawberry extract which includes anthocyanins and anthocyanidins which are not present in extracts using existing methods. Also, it can be noted that the extract prepared using the method of the invention contains sugars (including glucose, fructose, sucrose lactose, maltose, and galactose) above 0.2 g/100 g. This is in contrast extracts using existing methods in which sugars are present below 0.2 g/100 g. This can be important for the status of the extract since in some regulatory regimes extracts having above 0.2 g/100 g of sugars can be a juice.

### Example 10: Aroma Compounds from Strawberry Extract

Table 8 below shows measureable differences of key aroma compounds, obtained by a semi quantitative method (Dynamic HeadSpace (=DHS)/GC/MS) between a strawberry extract obtained using classical distillation process (classical extract) with the strawberry extract obtained using the method of the invention (termed ‘new strawberry eco-extract/juice’). The analytical comparison has been done at iso-sensory intensity and the conc. of molecules (in ppm) is relative to Ready to drink (RTD)

TABLE 8
Analytical comparison of key aroma compounds between
classical strawberry extract and eco-extract/juice

| | | Strawberry | | | | |
|---|---|---|---|---|---|---|
| | | classical | Strawberry | | | |
| | | extract | Eco- | | Strawberry | Strawberry |
| Chemical | | ppm, RTD | extract/juice | Sensory | Eco- | Classical |
| Class | CAS Name | Quantity | ppm, RTD | Attributes | extract/juice | Extract |
| ketones | 2,3 butanedione | nd | 0.0169 | Creamy | Present | absent |
| Saturated | 2-methylpropan- | 0.312 | nd | alcoholic, | absent | present |
| aliphatic | 1-ol | | | fermented | | |
| alcohols | 3 methyl-butanol | 0.516 | nd | notes | | |
| Sequiterpenic | 1,6,10- | nd | 0.0744 | floral | Present | absent |
| alcool | Dodecatrien-3-ol, | | | | | |
| (1,6,10- | 3,7,11-trimethyl- | | | | | |
| Dodecatrien- | | | | | | |
| 3-ol, 3,7,11- | | | | | | |
| trimethyl-) | | | | | | |
| and aliphatic | | | | | | |
| unsaturated | | | | | | |
| alcool (1,6- | | | | | | |
| Octadien-3- | | | | | | |
| ol, 3,7- | | | | | | |
| dimethyl-) | | | | | | |
| Aliphatic | (E)-2-HEXENAL | 0.4589 | 0.0220 | green | low | high |
| unsaturated | | | | Apple | | |
| aldehyde(2- | (E)-; Hexanal | 0.4589 | 0.0220 | | | |
| Hexenal, (E)-), | | | | | | |
| aliphatic | | | | | | |
| saturated | | | | | | |
| aldehyde | | | | | | |
| (hexanal) | | | | | | |
| Lactones | 2(3H)-Furanone, | 0.0102 | 0.0510 | Fleshy, | high | medium |
| | dihydro-5-octyl- | | | fruity | | |
| Alicyclic | 3(2H)-Furanone, | nd | 0.106 | Fruity, | present | absent |
| unsaturated | 4-hydroxy-2,5- | | | jammy | | |
| ketone | dimethyl- | | | | | |
| | 3(2H)-Furanone, | 0.0851 | 0.076 | | | |
| | 4-methoxy-2,5- | | | | | |
| | dimethyl- | | | | | |

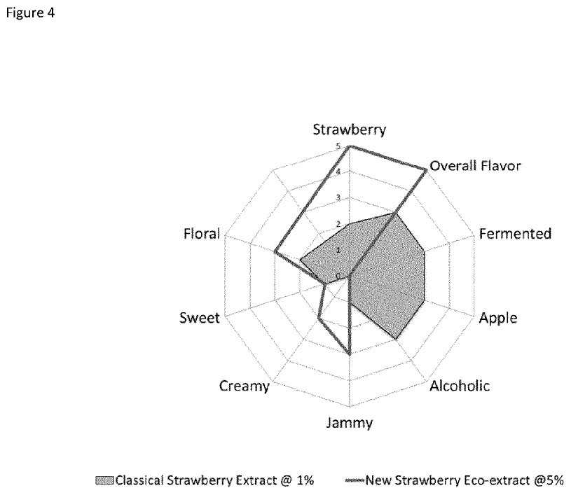

Iso intensity sensory profile of strawberry classical extract versus the new eco-extract in sweet-sour solution (5 Brix, 0.1% citric acid) is reported in the FIG. 4.

Here it can be seen that the strawberry extract obtained using the method of the invention (termed ‘new strawberry eco-extract/juice’) shows significant difference in the sensory profile to the classical extract. Notably such differences are in the increase in the overall flavour, jammy, and floral aspects of the profile. Other notable differences are in the reduction of fermented, apple and alcoholic profiles in the extract.

Indeed the strawberry extract obtained using the method of the invention has a much greater sensory profile in the overall ‘strawberry’ measurement than the classical extract.

The graph shows how the presence of key aroma compounds in the new eco-extract make the profile more strawberry authentic: fruity, creamy, jammy, floral, strawberry.

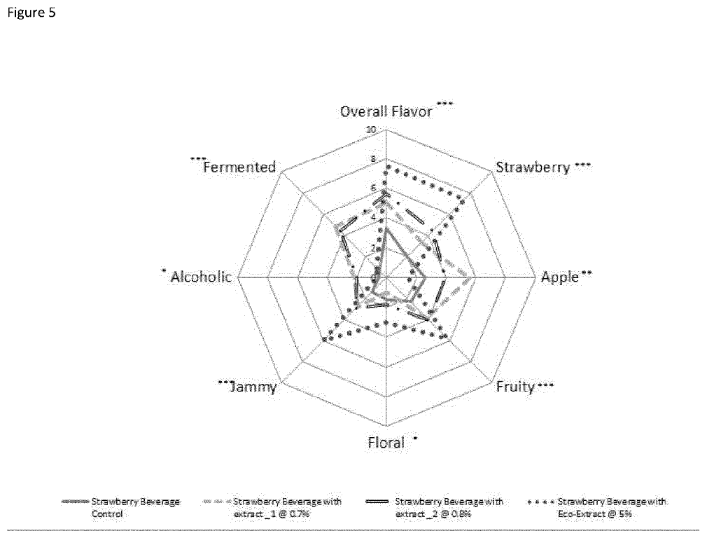

Another example of Iso intensity sensory profile of two Strawberry classical extracts versus the new eco-extract in a typical beverage application (BWAT Strawberry, Brix=5, pH=3.4) is reported in the FIG. 5.

Panel set-up: Objective: Sensory Profile; N. Panelists: 10 performing trained panelists; each sample was assessed twice Results from the ANOVA (Proba.): Significant difference between the samples at 90% (!)−95% (*)−99% (**) and 99.9% (***).

Extract_1 is perceived significantly more Apple, more Alcoholic and more Fermented than Eco Extract. Extract_2 is perceived significantly more Alcoholic and more Fermented than Eco Extract. Eco-Extract Org is perceived significantly stronger in Overall flavor, more Strawberry, more Jammy, more Fruity and more Floral than Extract_1 and Extract_2. Here it can be seen that the strawberry extract obtained using the method of the invention (termed ‘ Eco-Extract’) is more jammy, more fruity and more floral than the classical extract.

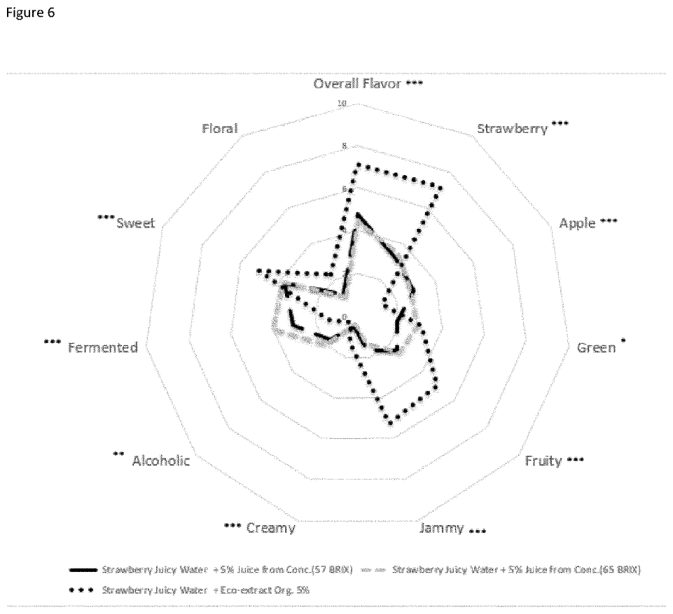

Another example of Iso intensity sensory profile of two Strawberry classical extracts versus the new eco-extract juice in a typical beverage application (BWAT Strawberry juicy, Brix=5, pH=3.4). The data is shown in FIG. 6.

Panel set-up: Objective: Describe the sensory properties of a Sample containing 5% juice from Conc. (57 Brix), one Sample containing 5% Juice from conc. (65 Brix) and one Sample containing Eco Extract/Juice applied in Strawberry Juicy Beverage. N. Panelists: 8 performing trained panelists; each sample was assessed twice; Intensity of 11 descriptors evaluated on linear scale (0=not perceptible; 10=very strong). Data analysis: the performance of the panel is validated according to several statistical tools which measure the level of repeatability, discrimination and consensus within the group. Identify significant differences amongst samples for each attribute Mixed model of Analysis of Variance (ANOVA) on the mean scores (Fixed effects=product; Random effect=panelists) Results from the ANOVA (Proba.): Significant difference between the samples at 90% (!)−95% (*)−99% (**) and 99.9% (***).

From FIG. 6 the following conclusion can be determined. (i) Strawberry Juicy water containing 5% juice from conc. (57 brix) is perceived significantly:—more Apple, more Alcoholic and more Fermented than Sample containing eco-extract/juice (a strawberry extract prepared from the method of the invention). (ii) Strawberry Juicy water containing 5% juice from conc. (65 brix) is perceived significantly:—more Apple, more Alcoholic and more Fermented than Beverage containing eco-extract/juice. (iii) Strawberry Juicy water containing Eco-Extract/juice is perceived significantly stronger in overall flavor, more atrawberry, more fruity, more jammy, more creamy, sweeter but less apple, less fermented and less alcoholic than two other samples.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

1. An extract of a plant biomass, wherein the extract comprises at least one anthocyanin.

2. The extract of claim 1, wherein the extract further comprises at least one anthocyanidin.

3. The extract of claim 1, wherein the concentration of the at least one anthocyanin in the extract is 1 ppm.

4. The extract of claim 2, wherein the concentration of the at least one anthocyanidin in the extract is 1 ppm.

5. The extract of claim 2, wherein the at least one anthocyanidin is selected from the group consisting of: cyanidin, delphinidin, malvinidin, peonidin, petunidin, and combinations thereof.

6. The extract of claim 1, wherein the at least one anthocyanin is selected from the group consisting of: cyanidin-3-arabinoside, cyanidin-3-galactoside, cyanidin-3-glucoside, delphinidin-3-arabinoside, delphinidin-3-galactoside, delphinidin-3-glucoside, malvinidin-3-arabinoside, malvinidin-3-galactoside, malvinidin-3-glucoside, peonidin-3-arabinoside, peonidin-3-galactoside, peonidin-3-glucoside, petunidin-3-arabinoside, petunidin-3-galactoside, petunidin-3-glucoside, and combinations thereof.

7. The extract of claim 1, wherein the at least one anthocyanin comprises cyanidin-3-glucoside, and the concentration of cyanidin-3-glucoside in the extract is 0.5 ppm.

8. The extract of claim 2, wherein the at least one anthocyanidin comprises peonidin, and the concentration of peonidin in the extract is 0.07 ppm.

9. The extract of claim 1, wherein the at least one anthocyanin comprises peonidin-3-galactoside, and the concentration of peonidin-3-galactoside in the extract is 0.2 ppm.

10. The extract of claim 1, wherein the at least one anthocyanin comprises peonidin-3-glucoside, and the concentration of peonidin-3-glucoside in the extract is 0.8 ppm.

11. The extract of claim 2, wherein the at least one anthocyanidin comprises petunidin, and the concentration of petunidin in the extract is 0.6 ppm.

12. The extract of claim 1, wherein the extract comprises 0.5 ppm cyanidin-3-glucoside, 0.07 ppm peonidin, 0.2 ppm peonidin-3-galactoside, 0.8 ppm peonidin-3-glucoside, and 0.6 ppm petunidin.

13. The extract of claim 1, further comprising glucose in an amount greater than 0.9 g/100 g of the extract.

14. The extract of claim 1, further comprising fructose in an amount from 1.1 to 1.5 g/100 g of the extract.

15. The extract of claim 1, further comprising sucrose in an amount from 0.2 to 1 g/100 g of the extract.

16. The extract of claim 1, further comprising reducing sugars in an amount from 2 to 2.5 g/100 g of the extract.

17. The extract of claim 1, further comprising mono and disaccharides in an amount from 2 to 3.5 g/100 g of the extract.

18. The extract of claim 1, wherein the plant biomass is strawberry.

19. The extract of claim 19 wherein the extract comprises: (i) 0.007 ppm to 0.027 ppm 2,3 butanedione, (ii) 0.05 ppm to 0.1 ppm 3,7,11-trimethyl-1,6,10-Dodecatrien-3-ol; (iii) 0.02 to 0.1 ppm dihydro-5-octyl-2(3H)-Furanone; (iv) 0.05 to 0.15 ppm 4-hydroxy-2,5-dimethyl-3(2H)-Furanone, (v) less than 0.3 ppm 2-methylpropan-1-ol; (vi) less than 0.5 ppm 3-methyl butanol; and (vii) less than 0.4 ppm 2-hexenal.

20. A perfumed article comprising the extract of claim 1, wherein the perfumed article is selected from the group consisting of: an aromatic water, a perfume, a cologne, a bath gel, a shower gel, a shampoo, a hair care product, a cosmetic preparation, a deodorant, an air freshener, a detergent, a fabric softener, and a household cleaner.

21. A flavored consumer product comprising the extract of claim 1, wherein the product is selected from the group consisting of: baked goods, dairy based products, dairy analogues, products based on fat and oil or emulsions thereof, milk products, confectionary products, desserts, chocolate and compound coatings, cereal products, non-alcoholic beverages, alcoholic beverages, and instant or ready-to-drink beverages.
